(19) United States
(12) Patent Application Publication
(10) Pub. No.: US 2021/0054086 A1
(43) Pub. Date: Feb. 25, 2021

(54) IMMUNE CELLS EXPRESSING A CHIMERIC ANTIGEN RECEPTOR

(71) Applicant: The General Hospital Corporation, Boston, MA (US)

(72) Inventors: Marcela V. Maus, Lexington, MA (US); Bryan Choi, Boston, MA (US)

(73) Assignee: The General Hospital Corporation, Boston, MA (US)

(21) Appl. No.: 16/961,189

(22) PCT Filed: Jan. 10, 2019
(86) PCT No.: PCT/US2019/013103

§ 371 Date: Jul. 9, 2020

Related U.S. Application Data
(63) Continuation of application No. PCT/US2018/013221, filed on Jan. 10, 2018.
(60) Provisional application No. 62/773,001, filed on Nov. 29, 2018.
(60) Provisional application No. 62/771,998, filed on Nov. 27, 2018.
(60) Provisional application No. 62/629,558, filed on Feb. 12, 2018.

(51) Int. Cl. C07K 16/28; C07K 14/725; C07K 14/705; A61K 35/17; A61P 35/00

(52) U.S. Cl. CPC C07K 16/2878; C07K 14/7051; C07K 14/70517; C07K 14/70521; C07K 14/70578; A61K 38/00; A61P 35/00; C07K 2319/02; C07K 2319/03; C07K 2319/33; A61K 35/17

(57) ABSTRACT

Described herein are methods for producing and utilizing T cells comprising chimeric antigen receptors (CAR) comprising two or more extracellular domains, each comprising a portion of the extracellular domain of a Tumor Necrosis Factor (TNF) superfamily receptor ligand, e.g., A PRoliferation-Inducing Ligand (APRIL). The CARs described herein are capable of targeting, e.g., B cell maturation antigen (BCMA) and/or transmembrane activator and CAML interactor (TACI). Additionally, the CAR T cells of this present invention overcome resistance to anti-BCMA targeted therapies and utilize dimerizing and trimerizing transmembrane domains for optimal function. Further, this invention is related to methods of treating cancer (e.g., multiple myeloma (MM)), plasma cell diseases or disorders, autoimmune diseases or disorders, or transplant rejection.

### CROSS-REFERENCE TO RELATED APPLICATIONS

This application claims benefit of International Patent Application No. PCT/US2018/013221, filed Jan. 10, 2018, and U.S. Provisional Application Nos. 62/629,558, filed Feb. 12, 2018, 62/771,998, filed Nov. 27, 2018, and 62/773,001, filed Nov. 29, 2018, the contents of which are incorporated herein by reference in their entirety.

### TECHNICAL FIELD OF THE INVENTION

The technology described herein relates to immunotherapy.

### SEQUENCE LISTING

The instant application contains a Sequence Listing which has been submitted electronically in ASCII format and is hereby incorporated by reference in its entirety. The ASCII copy, created on Jan. 10, 2018, is named 51295-012WO4_Sequence_Listing_1.10.19_ST25 and is 83,402 bytes in size.

### BACKGROUND OF THE INVENTION

Chimeric antigen receptors (CARs) provide a way to direct a cytotoxic T cell response to target cells expressing a selected target antigen, most often a tumor antigen or tumor-associated antigen. CARs are an adaptation of the T cell receptor (TCR), where the antigen binding domain is replaced, e.g., with the antigen-binding domain of an antibody that specifically binds the derived target antigen. Engagement of the target antigen on the surface of a target cell by a CAR expressed on a T cell (“CAR T cell”) promotes killing of the target cell.

Multiple myeloma accounts for about 13% of all hematological malignancies and is a result of the monoclonal expansion of plasma cells in the bone marrow, leading to the production of a paraprotein. Patients most often present painful bone lesions, anemia, hypercalcemia, and renal impairment. The standard of care for systemic treatment currently consists of chemotherapy, immunomodulatory drugs (IMiDs), proteasome inhibitors, and autologous stem cell transplantation. Despite therapeutic advances in recent years, relapses with an increasingly refractory disease remain common. Thus, there exists an unmet need for improved treatments for multiple myeloma.

### SUMMARY OF THE INVENTION

CAR T cells are a cutting edge therapeutic that shows great promise in treating cancer. Such therapeutics have proven particularly effective against various non-solid cancers, e.g., leukemias, lymphomas and myelomas. One of the greatest challenges with creating CAR T cells for a given disease or disorder is overcoming adverse reactions from off-target and systemic effects, such as cytokine release syndrome. While cytokine release syndrome is generally treatable, there is concern that the treatments for this complication may limit the efficacy and/or long term sustained effects of the CAR T cell treatment.

Another issue encountered in CAR T therapeutic designs is the escape of tumors through loss of the targeted antigen or tumor-associated factor recognized by the CAR. When a tumor down-regulates or otherwise loses cell surface expression of a targeted antigen or factor, it will no longer be efficiently attacked by CAR T cells designed to target that antigen or factor. This has been observed, for example in CAR T therapy targeting B cell maturation antigen (BCMA), which is expressed for example in B cell malignancies, leukemias, lymphomas and multiple myelomas.

Described herein are improvements in CAR design that avoid off-target effects and reduce the possibility for tumor escape by loss of target antigen.

Accordingly, one aspect of the invention described herein relates to a chimeric antigen receptor (CAR) polypeptide comprising: one or more extracellular domains comprising a portion of Tumor Necrosis Factor (TNF) superfamily receptor ligand; a hinge and transmembrane domain; a co-stimulatory domain; and an intracellular signaling domain. In one embodiment, an approach is described herein, demonstrated using BCMA-related proteins as example tumor-associated targets, that uses a single ligand that binds two different tumor-related antigens or factors. In some embodiments, a single ligand is fused to transmembrane and T cell receptor intracellular effector domains, optionally with co-stimulatory domains, essentially as for CARs known in the art. Having a ligand that binds two different tumor-associated antigens or factors, instead of a single antigen means that a CAR will not lose effectiveness if one or the other of the antigens or factors is down-regulated by cells of the tumor. This is illustrated herein using as a ligand a portion of the APRIL (A PRoliferation-Inducing Ligand) polypeptide, which binds with high affinity to both BCMA and TACI, another tumor-related antigen or factor. In some embodiments of any of the aspects described herein, the ligand oligomerizes (e.g., dimerizes or trimerizes), for example, by self-oligomerization. For example, in some embodiments, the ligand is a portion of a TNF superfamily receptor ligand. In some aspects, the CAR design includes more than one ligand (e.g., 2, 3, 4, 5, 6, 7, 8, 9, 10, or more ligands).

Accordingly, one aspect of the invention described herein relates to a CAR polypeptide comprising an extracellular domain comprising a portion of a TNF superfamily receptor ligand, which is N-terminal to the endogenous cleavage site, a hinge and transmembrane domain, a co-stimulatory domain, and an intracellular signaling domain. In one embodiment, the TNF superfamily receptor ligand is APRIL. In other embodiments, the TNF superfamily receptor ligand is TNF-alpha, lymphotoxin beta, OX40L, CD154, FasL, LIGHT, TL1A, CD70, Siva, CD153, 4-1BB ligand, TRAIL, RANKL, TWEAK, BAFF, CAMLG, LIGHT, NGF, BDNF, NT-3, NT-4, GITR ligand, TL1A, or EDA-A2.

Accordingly, one aspect of the invention described herein relates to a CAR polypeptide comprising an extracellular domain comprising a portion of APRIL, which is N-terminal to the endogenous cleavage site, a hinge and transmembrane domain, a co-stimulatory domain, and an intracellular signaling domain.

In one embodiment of any aspect, the CAR polypeptide further comprises a CD8 leader sequence. In one embodiment, the CD8 leader sequence comprises the sequence selected from SEQ ID NO: 20, 26, or 32, or is encoded by a nucleic acid comprising the sequence selected from SEQ ID NO: 2, 8, 9, or 14.

In one embodiment, the portion of APRIL comprises the sequence selected from SEQ ID NO: 21, 27, or 33, or is encoded by a nucleic acid comprising the sequence selected from SEQ ID NO: 3, 9, or 15. In one embodiment of any aspect, the portion of APRIL does not comprise a lysine-rich region of APRIL.

In one embodiment of any aspect, the hinge and transmembrane domain comprises the hinge and transmembrane domain of CD8 or 4-1 BB. In one embodiment, the CD8 hinge and transmembrane domain sequence comprises the sequence of SEQ ID NO: 22, or is encoded by a nucleic acid comprising the sequence of SEQ ID NO: 4. In one embodiment, the 4-1 BB hinge and transmembrane domain sequence is selected from SEQ ID NO: 28 or 34, or is encoded by a nucleic acid comprising the sequence of SEQ ID NO: 10 or 16.

In one embodiment of any aspect, the intracellular signaling domain comprises the signaling domain of CD3zeta, CD3 eta, or CD3 theta. In one embodiment, the CD3zeta intracellular signaling domain sequence is selected from SEQ ID NO: 24 or 30, or is encoded by a nucleic acid comprising the sequence of SEQ ID NO: 6 or 12. In one embodiment, the CD3 theta intracellular signaling domain sequence comprises the sequence of SEQ ID NO: 36, or is encoded by a nucleic acid comprising the sequence of SEQ ID NO: 18.

In one embodiment of any aspect, the co-stimulatory domain is 4-1 BB intracellular domain (ICD), CD28 ICD, CD27 ICD, ICOS ICD, or OX40 ICD. In one embodiment, the co-stimulatory domain is 4-1 BB ICD. In one embodiment, the 4-1 BB ICD sequence comprises a sequence selected from SEQ ID NO: 23, 29, or 35, or is encoded by a nucleic acid comprising the sequence of SEQ ID NO: 5, 11, or 17.

In one embodiment of any aspect, the CAR polypeptide comprises two or more (e.g., two or more, three or more, four or more, five or more, six or more, seven or more, eight or more, nine or more, or ten or more) extracellular domains comprising a portion of a TNF superfamily receptor ligand. In one embodiment, the CAR polypeptide comprises three extracellular domains comprising a portion of TNF superfamily receptor ligand.

Another aspect of the invention described herein relates to a CAR polypeptide comprising at least 95% identity with a sequence selected from SEQ ID NO: 19, 25, or 31, or that is encoded by a sequence comprising at least 95% identity with a sequence selected from SEQ ID NO: 1, 7, or 13. Another aspect of the invention described herein relates to a CAR polypeptide comprising a sequence selected from SEQ ID NO: 19, 25, or 31, or that is encoded by a sequence selected from SEQ ID NO: 1, 7, or 13.

Another aspect of the invention described herein relates to a CAR polypeptide comprising a sequence corresponding to a sequence selected from SEQ ID NO: 19, 25, or 31, or that is encoded by a sequence selected from SEQ ID NO: 1, 7, or 13.

Another aspect of the invention described herein relates to a polypeptide complex comprising two or more (e.g., two or more, three or more, four or more, five or more, six or more, seven or more, eight or more, nine or more, or ten or more) of any of the CAR polypeptides described herein. In one embodiment, the polypeptide complex comprises three of any of the CAR polypeptides described herein.

Another aspect of the invention described herein relates to a mammalian cell comprising; any of the CAR polypeptides described herein; a nucleic acid encoding any of the CAR polypeptides described herein; or any of the polypeptide complexes described herein.

In one embodiment of any aspect, the cell is a T cell. In one embodiment, the cell is a human cell. In one embodiment, the cell is obtained from an individual having or diagnosed as having cancer, a plasma cell disorder, or autoimmune disease.

Another aspect of the invention described herein relates to a method of treating cancer, a plasma cell disorder, amyloidosis, or an autoimmune disease in a subject, the method comprising: engineering a T cell to comprise any of the CAR polypeptides described herein on the T cell surface; administering the engineered T cell to the subject.

Another aspect of the invention described herein relates to a method of treating cancer, a plasma cell disorder, or an autoimmune disease in a subject, the method comprising administering a cell comprising any of the CAR polypeptides described herein, or a nucleic acid encoding any of the CAR polypeptides described herein.

In one embodiment of any aspect, the cancer is BAFF+, BCMA+ and/or TACI⁺. In one embodiment, wherein the cancer is multiple myeloma or smoldering myeloma.

In one embodiment of any aspect, the subject is further administered an anti-BCMA therapy. In one embodiment, the subject is resistant to anti-BCMA therapies.

In one embodiment of any aspect, the autoimmune disease is selected from the group consisting of hemophilia with antibodies to coagulation factors, myasthenia gravis, multiple sclerosis, and chronic graft v. host disease.

Another aspect of the technology described herein relates to a composition comprising a CAR polypeptide as described herein formulated for the treatment of cancer. In one embodiment, the composition further comprises a pharmaceutically acceptable carrier.

Another aspect of the technology described herein relates to a composition comprises a protein complex as described herein formulated for the treatment of cancer. In one embodiment, the composition further comprises a pharmaceutically acceptable carrier.

Another aspect of the technology described herein relates to a composition comprises a CAR T cell as described herein formulated for the treatment of cancer. In one embodiment, the composition further comprises a pharmaceutically acceptable carrier.

In still other aspects, the invention features a chimeric antigen receptor (CAR) polypeptide including: a) two or more extracellular domains, each including a portion of a Tumor Necrosis Factor (TNF) superfamily receptor ligand; b) a hinge and transmembrane domain; c) a co-stimulatory domain; and d) an intracellular signaling domain.

In another aspect, the invention features a chimeric antigen receptor (CAR) polypeptide including: a) two or more extracellular domains, each including a Tumor Necrosis Factor (TNF) superfamily receptor ligand or a portion thereof; b) a transmembrane domain; and c) an intracellular signaling domain. In some embodiments, the transmembrane domain includes a hinge/transmembrane domain. In some embodiments, the CAR polypeptide further includes one or more co-stimulatory domains.

In some embodiments, the TNF superfamily receptor ligand is A Proliferation-Inducing Ligand (APRIL). In further embodiments, the TNF superfamily receptor ligand is TNF-alpha, lymphotoxin beta, OX40 ligand (OX40L), CD154, Fas ligand (FasL), LIGHT, TNF-like ligand 1A (TL1A), CD70, Siva, CD153, 4-1 BB ligand (4-1 BBL), TNF-related apoptosis-inducing ligand (TRAIL), receptor activator of nuclear factor kappa-B ligand (RANKL), TNF-related weak inducer of apoptosis (TWEAK), B cell activating factor (BAFF), calcium modulating ligand (CAMLG or CAML), nerve growth factor (NGF), brain-derived neurotrophic factor (BDNF), neurotrophin-3 (NT-3), neurotrophin-4 (NT-4), glucocorticoid-induced TNF receptor (TNFR)-related protein (GITR) ligand, or ectodysplasin A2 (EDA-A2).

In some embodiments, the two or more extracellular domains each include a portion of the same TNF superfamily receptor ligand. In yet other embodiments, at least two of the extracellular domains each include a portion of different TNF superfamily receptor ligands.

In further embodiments, the two or more extracellular domains are connected to each other by one or more linker sequences, e.g., a linker sequence including an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 41, 58, 61, 62, or 63, or a linker sequence including the amino acid sequence of SEQ ID NO: 41, 58, 61, 62, or 63.

In some embodiments, the CAR polypeptide further includes a leader sequence, e.g., a CD8 leader sequence. In particular embodiments, the CD8 leader sequence includes a sequence having at least 90% sequence identity (e.g., 90%, 91%, 92%, 93%, 94%, 95%, 96%, 97%, 98%, or 99% sequence identity) to the sequence of SEQ ID NO: 20, 26, or 32, or includes the sequence of SEQ ID NO: 20, 26, or 32.

In some embodiments, the portion of APRIL does not include a lysine-rich region of APRIL (e.g., the lysine-rich sequence of SEQ ID NO: 38). In yet further embodiments, the portion of APRIL does not include a furin cleavage site (e.g., the furin cleavage site of SEQ ID NO: 66 or 67). In particular embodiments, the portion of APRIL includes a sequence having at least 90% sequence identity (e.g., 90%, 91%, 92%, 93%, 94%, 95%, 96%, 97%, 98%, or 99% sequence identity) to the sequence of SEQ ID NO: 21, 27, 33, or 40, or includes the sequence of SEQ ID NO: 21, 27, 33, or 40.

In further embodiments, the hinge and transmembrane domain includes the hinge and transmembrane domain of CD28, CD8, or 4-1 BB. In particular embodiments, the CD8 hinge and transmembrane domain includes an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 22 or 42, or includes the amino acid sequence of SEQ ID NO: 22 or 42. In other embodiments, the 4-1 BB hinge and transmembrane domain includes an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 28 or 34, or includes the amino acid sequence of SEQ ID NO: 28 or 34.

In some embodiments, the intracellular signaling domain includes the intracellular signaling domain of CD3ζ, CD3ε, or CD3θ. In some embodiments, the CD3 intracellular signaling domain includes an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 24, 30, or 44, or includes the amino acid sequence of SEQ ID NO: 24, 30, or 44. In other embodiments, the CD3θ intracellular signaling domain includes an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 36, or includes the amino acid sequence of SEQ ID NO: 36.

In further embodiments, the co-stimulatory domain includes the intracellular domain of 4-1 BB, CD28, CD27, ICOS, or OX40. In particular embodiments, the co-stimulatory domain is the intracellular domain of 4-1 BB, e.g., wherein the intracellular domain of 4-1 BB includes an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 23, 29, 35, or 43, or includes the amino acid sequence of SEQ ID NO: 23, 29, 35, or 43.

In particular embodiments, the CAR polypeptide includes three extracellular domains, each including a portion of a TNF superfamily receptor ligand (e.g., three APRIL domains, also referred to herein as TriPRIL).

In another aspect, the invention features a CAR polypeptide including at least 95% sequence identity (e.g., 95%, 96%, 97%, 98%, or 99% sequence identity) with the sequence of SEQ ID NO: 39, 57, 64, or 65, or that is encoded by a sequence including at least 95% sequence identity (e.g., 95%, 96%, 97%, 98%, or 99% sequence identity) with the sequence of SEQ ID NO: 45.

In another aspect, the invention features a CAR polypeptide including the sequence of SEQ ID NO: 39, 57, 64, or 65, or that is encoded by the sequence of SEQ ID NO: 45.

In another aspect, the invention features a CAR polypeptide including a sequence corresponding to the sequence of SEQ ID NO: 39, 57, 64, or 65, or that is encoded by a sequence of SEQ ID NO: 45.

In another aspect, the invention features a polypeptide complex including two or more of the CAR polypeptides of any one of the preceding aspects. In some embodiments, the polypeptide complex includes three CAR polypeptides of any one of any one of the preceding aspects.

In another aspect, the invention features a mammalian cell including: a) the CAR polypeptide of any one of the preceding aspects; b) a nucleic acid encoding the CAR polypeptide of any one of the preceding aspects; or c) the polypeptide complex including two or more (e.g., three) of the CAR polypeptides of any one of the preceding aspects.

In some embodiments, the cell is an immune cell such as a T cell or a natural killer (NK) cell. In some embodiments, the cell is a human cell. In some embodiments, the cell is obtained from an individual having or diagnosed as having cancer, a plasma cell disorder, or an autoimmune disease or disorder.

In another aspect, the invention features a method of treating a cancer, a plasma cell disorder, amyloidosis, an autoimmune disease or disorder, or transplant rejection in a subject, the method including: a) engineering a T or NK cell to include the CAR of any one of the preceding aspects on the T or NK cell surface; and b) administering the engineered T or NK cell to the subject.

In another aspect, the invention features a method of treating a subject, the method including administering the cell of any one of the preceding aspects to the subject. In some embodiments, the subject has a cancer, a plasma cell disorder, an autoimmune disease or disorder, or transplant rejection.

In some embodiments, the cancer is BAFF+, B cell maturation antigen (BCMA)+ and/or transmembrane activator and CAML interactor (TACI)+. In some embodiments, the subject is further administered an anti-BCMA therapy. In further embodiments, the subject is resistant to anti-BCMA therapies.

In particular embodiments, the cancer is multiple myeloma, smoldering myeloma, or Waldenstrom's macroglobulenemia.

In yet other embodiments, the autoimmune disease is selected from the group consisting of hemophilia with antibodies to coagulation factors, myasthenia gravis, multiple sclerosis, and chronic graft versus host disease.

In further embodiments, the subject has high levels of anti-human leukocyte antigen (HLA) antibodies.

In another aspect, the invention features a composition including the CAR polypeptide, the polypeptide complex, or the cell of any one of the preceding aspects formulated for the treatment of cancer. In some embodiments, the composition further includes a pharmaceutically acceptable carrier.

In other aspects, the invention features a method of treating a cancer, a plasma cell disorder, an autoimmune disease or disorder, or transplant rejection in a subject resistant to anti-BCMA therapy, the method including administering to the subject an immune cell including a CAR and/or a polynucleotide encoding the CAR, wherein the CAR includes an extracellular target-binding domain including two or more APRIL domains.

In some embodiments, the cancer includes cells expressing BCMA and/or TACI. In some embodiments, the cancer includes cells with reduced BCMA expression. In some embodiments, the cancer is a myeloma (e.g., multiple myeloma or smoldering myeloma) or Waldenstrom's macroglobulinemia.

In other embodiments, the plasma cell disorder is amyloidosis.

In some embodiments, the autoimmune disease or disorder is selected from the group consisting of hemophilia with antibodies to coagulation factors, myasthenia gravis, multiple sclerosis, and chronic graft versus host disease.

In further embodiments, the subject has high levels of anti-HLA antibodies.

In some embodiments, the CAR includes a transmembrane domain and an intracellular signaling domain. In further embodiments, the CAR further includes one or more co-stimulatory domains.

In some embodiments, the transmembrane domain includes a hinge/transmembrane domain. In some embodiments, the hinge/transmembrane domain includes the hinge/transmembrane domain of an immunoglobulin-like protein (e.g., IgA, IgD, IgE, IgG, or IgM), CD28, CD8, or 4-1 BB. In specific embodiments, the hinge/transmembrane domain includes the hinge/transmembrane domain of CD8, optionally including an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 22 or 42, or including the amino acid sequence of SEQ ID NO: 22 or 42. In other embodiments, the hinge/transmembrane domain includes the hinge/transmembrane domain of 4-1 BB, optionally including an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 28 or 34, or including the amino acid sequence of SEQ ID NO: 28 or 34.

In further embodiments, the intracellular signaling domain includes the intracellular signaling domain of TCFζ, FcRγ, FcRβ, CD3γ, CD3θ, CD3δ, CD3ε, CD3η, CD3 ζ, CD22, CD79a, CD79b, or CD66d. In particular embodiments, the intracellular signaling domain includes the intracellular signaling domain of CD3ζ, optionally including an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 24, 30, or 44, or including the amino acid sequence of SEQ ID NO: 24, 30, or 44. In other embodiments, the intracellular signaling domain includes the intracellular signaling domain of CD3θ, optionally including an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 36, or including the amino acid sequence of SEQ ID NO: 36.

In some embodiments, the co-stimulatory domain includes the co-stimulatory domain of 4-1 BB, CD27, CD28, or OX40. In specific embodiments, the co-stimulatory domain includes the co-stimulatory domain of 4-1 BB, optionally including an amino acid sequence having at least 90% sequence identity (e.g., 90%, 91%, 92%, 93%, 94%, 95%, 96%, 97%, 98%, or 99% sequence identity) to the amino acid sequence of ID NO: 23, 29, 35, or 43, or including the amino acid sequence of SEQ ID NO: 23, 29, 35, or 43.

In some embodiments, each APRIL domain includes an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 21, 27, 33, or 40, or includes the amino acid sequence of SEQ ID NO: 21, 27, 33, or 40.

In further embodiments, the APRIL domains are connected to each other by one or more linker sequences. In some embodiments, the linker sequence includes an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 41, 58, 61, 62, or 63, or includes the amino acid sequence of SEQ ID NO: 41, 58, 61, 62, or 63.

In particular embodiments, the extracellular target-binding domain includes three APRIL domains. In some embodiments, the extracellular target-binding domain includes an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 56 or 59, or includes the amino acid sequence of SEQ ID NO: 56 or 59.

In some embodiments, the CAR includes an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 39, 57, 64, or 65, or includes the amino acid sequence of SEQ ID NO: 39, 57, 64, or 65.

In some embodiments, the polynucleotide encoding the CAR further includes a suicide gene. In further embodiments, the polynucleotide encoding the CAR further includes a sequence encoding a signal sequence.

In further embodiments, the immune cell is a T or NK cell, e.g., a human cell.

In another aspect, the invention features a CAR including an extracellular target-binding domain including three APRIL domains.

In some embodiments, the CAR includes a transmembrane domain and an intracellular signaling domain. In further embodiments, the CAR further includes one or more co-stimulatory domains.

In some embodiments, the transmembrane domain includes a hinge/transmembrane domain. In some embodiments, the hinge/transmembrane domain includes the hinge/transmembrane domain of an immunoglobulin-like protein (e.g., IgA, IgD, IgE, IgG, or IgM), CD28, CD8, or 4-1 BB. In specific embodiments, the hinge/transmembrane domain includes the hinge/transmembrane domain of CD8, optionally including an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 22 or 42, or including the amino acid sequence of SEQ ID NO: 22 or 42. In other embodiments, the hinge/transmembrane domain includes the hinge/transmembrane domain of 4-1 BB, optionally including an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 28 or 34, or including the amino acid sequence of SEQ ID NO: 28 or 34.

In further embodiments, the intracellular signaling domain includes the intracellular signaling domain of TCFζ, FcRγ, FcRβ, CD3γ, CD3θ, CD3δ, CD3ε, CD3η, CD3ζ, CD22, CD79a, CD79b, or CD66d. In particular embodiments, the intracellular signaling domain includes the intracellular signaling domain of CD3ζ, optionally including an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 24, 30, or 44, or including the amino acid sequence of SEQ ID NO: 24, 30, or 44. In other embodiments, the intracellular signaling domain includes the intracellular signaling domain of CD3θ, optionally including an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 36, or including the amino acid sequence of SEQ ID NO: 36.

In some embodiments, the co-stimulatory domain includes the co-stimulatory domain of 4-1 BB, CD27, CD28, or OX40. In specific embodiments, the co-stimulatory domain includes the co-stimulatory domain of 4-1 BB, optionally including an amino acid sequence having at least 90% sequence identity (e.g., 90%, 91%, 92%, 93%, 94%, 95%, 96%, 97%, 98%, or 99% sequence identity) to the amino acid sequence of ID NO: 23, 29, 35, or 43, or including the amino acid sequence of SEQ ID NO: 23, 29, 35, or 43.

In some embodiments, each APRIL domain includes an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 21, 27, 33, or 40, or includes the amino acid sequence of SEQ ID NO: 21, 27, 33, or 40.

In further embodiments, the APRIL domains are connected to each other by one or more linker sequences. In some embodiments, the linker sequence includes an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 41, 58, 61, 62, or 63, or includes the amino acid sequence of SEQ ID NO: 41, 58, 61, 62, or 63.

In particular embodiments, the extracellular target-binding domain includes three APRIL domains. In some embodiments, the extracellular target-binding domain includes an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 56 or 59, or includes the amino acid sequence of SEQ ID NO: 56 or 59.

In particular embodiments, the CAR includes an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 39, 57, 64, or 65, or includes the amino acid sequence of SEQ ID NO: 39, 57, 64, or 65.

In another aspect, the invention features a CAR including an amino acid sequence having at least 90% sequence identity (e.g., 90%, 91%, 92%, 93%, 94%, 95%, 96%, 97%, 98%, or 99% sequence identity) to the amino acid sequence of SEQ ID NO: 39, 57, 64, or 65.

In another aspect, the invention features a CAR including the amino acid sequence of SEQ ID NO: 39, 57, 64, or 65.

In another aspect, the invention features a polynucleotide encoding the CAR of any one of the preceding aspects. In some embodiments, the polynucleotide further includes a suicide gene. In further embodiments, the polynucleotide further includes a sequence encoding a signal sequence.

In another aspect, the invention features an immune cell including the CAR and/or the polynucleotide of any one of the preceding aspects.

In some embodiments, the immune cell is a T or NK cell, e.g., a human cell.

In another aspect, the invention features a pharmaceutical composition including the CAR, the polynucleotide, and/or the immune cell of any one of the preceding aspects.

In another aspect, the invention features a method of treating a cancer, a plasma cell disorder, an autoimmune disease or disorder, or transplant rejection in a subject, the method including administering to the subject the immune cell and/or the pharmaceutical composition of the preceding aspects.

In some embodiments, the cancer includes cells expressing BCMA and/or TACI. In some embodiments, the cancer includes cells with reduced BCMA expression. In further embodiments, the subject is resistant to anti-BCMA therapy. In particular embodiments, the cancer is a myeloma (e.g., multiple myeloma or smoldering myeloma) or Waldenstrom's macroglobulinemia.

In other embodiments, the plasma cell disorder is amyloidosis. In some embodiments, the autoimmune disease or disorder is selected from the group consisting of hemophilia with antibodies to coagulation factors, myasthenia gravis, multiple sclerosis, and chronic graft versus host disease. In further embodiments, the subject has high levels of anti-HLA antibodies.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

The term “TNF superfamily receptor ligand” refers to a ligand that binds to a tumor necrosis factor (TNF) superfamily receptor. TNF superfamily receptor ligands can be active as non-covalent oligomers (e.g., trimers). In some embodiments, a TNF superfamily receptor ligand is active as a homooligomer (e.g., a homotrimer). However, some TNF superfamily receptor ligands can be active as a heterooligomer (e.g., a heterotrimer), including BAFF, which can form a heterooligomer with APRIL. In some embodiments, the TNF superfamily receptor ligand is one that is described in Aggarwal, Nat. Rev. Immunol. 3:745-756, 2003 or Croft et al. Nat. Rev. Immunol. 9(4):271-285, 2009. In some embodiments, the TNF superfamily receptor ligand is TNF-alpha, lymphotoxin beta, OX40 ligand (OX40L), CD154, Fas ligand (FasL), LIGHT, TNF-like ligand 1 A (TL1A), CD70, Siva, CD153, 4-1 BB ligand (4-1 BBL), TNF-related apoptosis-inducing ligand (TRAIL), receptor activator of nuclear factor kappa-B ligand (RANKL), TNF-related weak inducer of apoptosis (TWEAK), B cell activating factor (BAFF), calcium modulating ligand (CAMLG or CAML), LIGHT, nerve growth factor (NGF), brain-derived neurotrophic factor (BDNF), neurotrophin-3 (NT-3), neurotrophin-4 (NT-4), glucocorticoid-induced TNF receptor (TNFR)-related protein (GITR) ligand, TL1A, or ectodysplasin A2 (EDA-A2). In some embodiments, the TNF superfamily receptor ligand binds to a TNF superfamily receptor described in Aggarwal, supra, or Croft et al, supra, including, e.g., tumor necrosis factor receptor 1 (TNFR1), tumor necrosis factor receptor 2 (TNFR2), CD95, decoy receptor 3 (DCR3), death receptor 3 (DR3), death receptor 4 (DR4), death receptor 5 (DR5), decoy receptor 1 (DCR1), decoy receptor 2 (DCR2), death receptor 6 (DR6), ectodysplasin A receptor (EDAR), nerve growth factor receptor (NGFR), osteoprotegerin (OPG), receptor activator of nuclear factor kappa-B (RANK), lymphotoxin beta receptor (LTbetaR), fibroblast growth factor-inducible 14 (FN14), herpesvirus entry mediator (HVEM), CD27, CD30, CD40, 4-1 BB, OX40, GITR, B cell maturation antigen (BCMA), transmembrane activator and CAML interactor (TACI), BAFF receptor (BAFFR), X-linked ectodysplasin A2 receptor (XEDAR), TROY, or receptor expressed in lymphoid tissues (RELT).

The term “portion” refers to a part of a polypeptide, e.g., a TNF superfamily receptor ligand (e.g., APRIL). In some embodiments, a portion of a TNF superfamily receptor ligand is N-terminal to the endogenous cleavage site, and comprises at least the TNF-like domain. In some embodiments, a portion of a TNF superfamily receptor ligand is capable of oligomerization (e.g., dimerization or trimerization). The oligomerization may be homooligomerizaion or heterooligomerization. In particular embodiments, the portion of a TNF superfamily receptor ligand is a portion of APRIL, such as a truncated APRIL. Exemplary truncated APRILs are shown in SEQ ID NO: 21, 27, 33, and 40.

As used herein, the term “APRIL domain” refers to the full-length sequence of APRIL, or a portion thereof, which is incorporated into a CAR polypeptide. An “APRIL domain” also refers to an amino acid sequence having at least 1, 2, 3, 4, 5, 6, 7, 8, 9, 10, 11, 12, 13, 14, 15, 16, 17, 18, 19, or 20 amino acid substitution(s) or deletion(s) relative to the full-length sequence of APRIL, or a portion thereof, or at least 1, 2, 3, 4, 5, 6, 7, 8, 9, 10, 11, 12, 13, 14, 15, 16, 17, 18, 19, or 20 amino acid substitution(s) or deletion(s) relative to the sequence of SEQ ID NO: 21, 27, 33, 40, or 60. An “APRIL domain” further includes an amino acid sequence having at least 80%, at least 85%, at least 90%, at least 91%, at least 92%, at least 93%, at least 94%, at least 95%, at least 96%, at least 97%, at least 98%, or at least 99% sequence identity to the full-length sequence of APRIL, or a portion thereof, or having at least 80%, at least 85%, at least 90%, at least 91%, at least 92%, at least 93%, at least 94%, at least 95%, at least 96%, at least 97%, at least 98%, or at least 99% to the APRIL sequence of SEQ ID NO: 21, 27, 33, 40, or 60.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. Where applicable, a decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include, for example, chimpanzees, cynomologous monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of disease e.g., cancer. A subject can be male or female.

A subject can be one who has been previously diagnosed with or identified as suffering from or having a condition in need of treatment (e.g. leukemia or another type of cancer, among others) or one or more complications related to such a condition, and optionally, have already undergone treatment for the condition or the one or more complications related to the condition. Alternatively, a subject can also be one who has not been previously diagnosed as having such condition or related complications. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

A “disease” is a state of health of an animal, for example a human, wherein the animal cannot maintain homeostasis, and wherein if the disease is not ameliorated, then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “tumor antigen” and “cancer antigen” are used interchangeably to refer to antigens which are differentially expressed by cancer cells and can thereby be exploited in order to target cancer cells. Cancer antigens are antigens which can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), and fusion proteins resulting from internal deletions or chromosomal translocations. Still other cancer antigens can be encoded by viral genes such as those carried on RNA and DNA tumor viruses. Many tumor antigens have been defined in terms of multiple solid tumors: MAGE 1, 2, & 3, defined by immunity; MART-1/Melan-A, gp100, carcinoembryonic antigen (CEA), HER2, mucins (i.e., MUC-1), prostate-specific antigen (PSA), and prostatic acid phosphatase (PAP). In addition, viral proteins such as some encoded by hepatitis B (HBV), Epstein-Barr (EBV), and human papilloma (HPV) have been shown to be important in the development of hepatocellular carcinoma, lymphoma, and cervical cancer, respectively.

As used herein, the term “chimeric” refers to the product of the fusion of portions of at least two or more different polynucleotide molecules. In one embodiment, the term “chimeric” refers to a gene expression element produced through the manipulation of known elements or other polynucleotide molecules.

In some embodiments, “activation” can refer to the state of a T cell that has been sufficiently stimulated to induce detectable cellular proliferation. In some embodiments activation can refer to induced cytokine production. In other embodiments, activation can refer to detectable effector functions. At a minimum, an “activated T cell” as used herein is a proliferative T cell.

As used herein, the terms “specific binding” and “specifically binds” refer to a physical interaction between two molecules, compounds, cells and/or particles wherein the first entity binds to the second, target, entity with greater specificity and affinity than it binds to a third entity which is a non-target. In some embodiments, specific binding can refer to an affinity of the first entity for the second target, entity, which is at least 10 times, at least 50 times, at least 100 times, at least 500 times, at least 1000 times or more greater than the affinity for the third nontarget entity under the same conditions. A reagent specific for a given target is one that exhibits specific binding for that target under the conditions of the assay being utilized. A non-limiting example includes an antibody, or a ligand, which recognizes and binds with a cognate binding partner (for example, a stimulatory and/or costimulatory molecule present on a T cell) protein.

A “stimulatory ligand,” as used herein, refers to a ligand that when present on an antigen presenting cell (APC e.g., a macrophage, a dendritic cell, a B-cell, an artificial APC, and the like) can specifically bind with a cognate binding partner (referred to herein as a “stimulatory molecule” or “co-stimulatory molecule”) on a T cell, thereby mediating a primary response by the T cell, including, but not limited to, proliferation, activation, initiation of an immune response, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

A “stimulatory molecule,” as the term is used herein, means a molecule on a T cell that specifically binds with a cognate stimulatory ligand present on an antigen presenting cell.

“Co-stimulatory ligand,” as the term is used herein, includes a molecule on an APC that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, 4-1 BBL, OX40L, CD7, B7-1 (CD80), B7-2 (CD86), PD-L1, PD-L2, inducible COStimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll-like receptor and a ligand that specifically binds with B7-H3. A co-stimulatory ligand also can include, but is not limited to, an antibody that specifically binds with a co-stimulatory molecule present on a T cell, such as, but not limited to, CD27, CD28, 4-1 BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

4-1 BBL is a type 2 transmembrane glycoprotein belonging to the TNFR/TNF ligand superfamily. 4-1 BBL is a co-stimulatory ligand that binds receptor 4-1 BB (CD137) expressed on T cell. 4-1 BBL is expressed on professional APCs including dendritic cells, macrophages, and activated B cells. 4-1 BBL sequences are known for a number of species, e.g., human 4-1 BBL, also known as TNFSF9 (NCBI Gene ID: 8744) polypeptide (e.g., NCBI Ref Seq NP_003802.1) and mRNA (e.g., NCBI Ref Seq NM_003811.3). 4-1 BBL can refer to human 4-1 BBL, including naturally occurring variants, molecules, and alleles thereof. In some embodiments of any of the aspects, e.g., in veterinary applications, 4-1 BBL can refer to the 4-1 BBL of, e.g., dog, cat, cow, horse, pig, and the like. Homologs and/or orthologs of human 4-1 BBL are readily identified for such species by one of skill in the art, e.g., using the NCBI ortholog search function or searching available sequence data for a given species for sequence similar to a reference 4-1 BBL sequence.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA, a Toll-like receptor, CD27, CD28, 4-1 BB, OX40, CD30, CD40, PD-1, ICOS, lymphocyte function-associated antigen-1 (LFA-1), CD2, CD7, LIGHT, NKG2C, B7-H3, and CD83.

In one embodiment, the term “engineered” and its grammatical equivalents as used herein can refer to one or more human-designed alterations of a nucleic acid, e.g., the nucleic acid within an organism's genome. In another embodiment, engineered can refer to alterations, additions, and/or deletion of genes. An “engineered cell” can refer to a cell with an added, deleted and/or altered gene.

The term “cell” or “engineered cell” and their grammatical equivalents as used herein can refer to a cell of human or non-human animal origin.

As used herein, the term “operably linked” refers to a first polynucleotide molecule, such as a promoter, connected with a second transcribable polynucleotide molecule, such as a gene of interest, where the polynucleotide molecules are so arranged that the first polynucleotide molecule affects the function of the second polynucleotide molecule. The two polynucleotide molecules may or may not be part of a single contiguous polynucleotide molecule and may or may not be adjacent. For example, a promoter is operably linked to a gene of interest if the promoter regulates or mediates transcription of the gene of interest in a cell.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of ordinary skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g., ligand-mediated receptor activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, a polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to an assay known in the art or described below herein. A functional fragment can comprise conservative substitutions of the sequences disclosed herein.

In some embodiments, a polypeptide described herein can be a variant of a polypeptide or molecule as described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity of the non-variant polypeptide. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are well established and include, for example, those disclosed by Walder et al. (Gene 42:133, 1986); Bauer et al. (Gene 37:73, 1985); Craik (BioTechniques, Jan. 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of a polypeptide also can be substituted, generally with serine, to improve the oxidative stability of the molecule and prevent aberrant crosslinking. Conversely, cysteine bond(s) can be added to a polypeptide to improve its stability or facilitate oligomerization.

As used herein, the term “DNA” is defined as deoxyribonucleic acid. The term “polynucleotide” is used herein interchangeably with “nucleic acid” to indicate a polymer of nucleosides. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

The term “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide.” Exemplary modifications include glycosylation and palmitoylation. Polypeptides can be purified from natural sources, produced using recombinant DNA technology or synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

In some embodiments, a nucleic acid encoding a polypeptide as described herein (e.g. a CAR polypeptide) is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′ UTR) or “leader” sequences and 3′ UTR or “trailer” sequences, as well as intervening sequences (introns) between individual coding segments (exons).

As used herein, a “signal peptide” or “signal sequence” refers to a peptide at the N-terminus of a newly synthesized protein that serves to direct the nascent protein into the endoplasmic reticulum. In some embodiments, the signal peptide is a CD8 signal peptide.

As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra-chromosomal DNA thereby eliminating potential effects of chromosomal integration.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. acute lymphoblastic leukemia or other cancer, disease, or disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier, e.g., a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier in which the active ingredient would not be found to occur in nature.

As used herein, the term “administering,” refers to the placement of a therapeutic or pharmaceutical composition as disclosed herein into a subject by a method or route which results in at least partial delivery of the agent at a desired site. Pharmaceutical compositions comprising agents as disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the technology.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined within the description of the various aspects and embodiments of the technology of the following.

The methods and constructs described herein provide several advantages. For example, fully human CARs including TriPRIL CAR affords a lower incidence of immunogenicity and rejection of the CAR T cell infusion. Furthermore, TriPRIL CAR T cells expand less rapidly in mice, which may result in a lower incidence of cytokine release syndrome associated with rapid CAR T cell expansion. TriPRIL CAR constructs also maintain a trimeric structure of the TNF superfamily member, but with the canonical stable configuration of a CAR backbone.

Other features and advantages of the invention will be apparent from the following description of the preferred embodiments thereof, and from the claims.

### BRIEF DESCRIPTION OF THE DRAWINGS

FIG. 1 shows a schematic comparison of scFv-based anti-BCMA CAR vs. APRIL anti-BCMA/TACI CAR.

FIG. 2 shows a schematic diagram of certain embodiments of the CARs described herein. It is expected that use of the 4-1 BB transmembrane domain is more likely to promote trimerization.

FIG. 3 shows target surface expression in the indicated cell types.

FIG. 4 shows a growth curve for cells expressing APRIL or BCMA CARs.

FIG. 5A shows the CAR-T cell transduction efficiency of APRIL CAR. FIG. 5B depicts the CAR-T cell transduction efficiency of BCMA CAR. X-axis is mCherry, y-axis is side scatter. Cells are gated on live CD3+ T cells.

FIG. 6 shows the results of a killing assay comparing APRIL and BCMA CARs.

FIG. 7 shows the results of an activation assay comparing APRIL and BCMA CARs. CAR-mediated T cell activation was tested in a Jurkat cell line expressing luciferase behind the NFAT promoter (JNL). JNL cells were lentivirally transduced with CARs as indicated and exposed to the targets indicated on the x-axis for several hours. Light emission was measured (relative Light units, y-axis).

FIG. 8 shows the level of expression of BCMA and TACI in the indicated multiple myeloma cell lines.

FIG. 9 shows the expression of BCMA and TACI in engineered cell lines.

FIG. 10 shows schematics of several APRIL and BCMA CARs and their transduction efficiencies.

FIG. 11 shows a graph demonstrating that BCMA and APRIL CARs expand upon repeated stimulation with RPMI8226^{PARENTAL}.

FIG. 12 shows a graph of APRIL-CAR killing of BCMA and TACI expressing cell lines.

FIG. 13 shows specific activation of APRIL-CAR.

FIG. 14 shows that BCMA and APRIL CARs degranulate in response to stimulation with RPMI8226^{PARENTAL}.

FIG. 15 shows the cytokine profile of APRIL-CART cells.

FIG. 16 shows a schematic diagram of the exemplary TriPRIL construct.

FIG. 17 shows CAR-T cell transduction efficiency of TriPRIL CAR. The x-axis shows mCherry signal, and the y-axis is side scatter. The top panel shows control untransduced (UTD) cells, and the bottom panel shows cells transduced with the TriPRIL CAR.

FIG. 18 shows the results of a cell killing assay using TriPRIL CAR T cells.

FIG. 19 shows BCMA and TACI expression on plasma cells obtained from multiple myeloma patients (n=25).

FIG. 20 shows the transduction efficiency of APRIL-based CARs in primary human T cells (MOI=5, n=3).

FIG. 21 shows BCMA and TACI expression on target cell lines RPMI8226, MM.1S, K562-BCMA, and K562-TACI.

FIGS. 22A and 22B show the results of an in vitro CD69 activation assay with BCMA, APRIL, and TriPRIL CAR T cells. FIG. 22A shows the gating strategy. FIG. 22B shows activation based on percent of CD69 expression after co-culture with target cells for 12 hours.

FIG. 23 shows the results of an in vitro killing assay with BCMA CAR, APRIL-CD8 CAR, and APRIL-4-1 BB CAR.

FIG. 24 shows the results of an in vitro killing assay with BCMA-CAR, APRIL-CD8 CAR, and TriPRIL CAR.

FIG. 25A shows the experimental design of an in vivo experiment with MM.1 S cells in NOD scid gamma (NSG) mice.

FIG. 25B shows bioluminescence imaging of tumor burden on NSG mice at the indicated time points.

FIG. 25C shows quantification of tumor burden in NSG mice at the indicated time points.

FIG. 25D shows the absolute number of CD3+/mCherry positive cells in blood 6.5 days after injection of CAR T or UTD cells.

FIG. 26A shows BCMA and TACI expression on plasma cells obtained from multiple myeloma patients.

FIG. 26B shows the levels of expression of BCMA (PE) and TACI (APC) on human multiple myeloma cell lines RPMI8226 and MM.1 S, and K562 cells modified to express either BCMA or TACI.

FIG. 26C shows a rendition of the binding of various CARs.

FIG. 26D shows the design of second-generation CARs targeting BCMA individually and BCMA and TACI concurrently.

FIG. 27A shows the affinity of BCMA scFv, APRIL-4-1 BB, and TriPRIL CARs for soluble BCMA (sBCMA) and soluble TACI (sTACI).

FIGS. 27B-27E show the cytotoxicity of the different CAR constructs in response to BCMA- and/or TACI-expressing target cells MM.1 S (FIG. 27B), RPMI8226 (FIG. 27C), K562-BCMA (FIG. 27D), and K562-TACI (FIG. 27E).

FIGS. 27F and 27G show the degranulation (FIG. 27F) and activation (FIG. 27G) of the different CAR constructs in response to BCMA- and/or TACI-expressing target cells.

FIGS. 27H and 271 show the long-term proliferation of different CAR T cells induced by repeated antigen stimulation with either BCMA (FIG. 27H) or TACI (FIG. 27I).

FIG. 27J shows the cytokine production by the different CAR T cells upon co-culture with human MM.1 S myeloma cells.

FIG. 28A shows the experimental design for in vivo testing of CAR T cells.

FIG. 28B shows representative bioluminescence imaging of myeloma xenografts over time.

FIG. 28C shows the quantification of flux (photons/second) in the four groups at the indicated time points.

FIG. 28D shows the persistence of CAR T cells measured in the peripheral blood by flow cytometry.

FIG. 29A shows BCMA and TACI expression of MM.1S myeloma cells generated with a CRISPR-mediated BCMA knockout.

FIG. 29B shows population doubling of MM.1 S, MM.1S BCMA KO I, and MM.1S BCMA KO II cell lines.

FIG. 29C shows specific lysis of MM.1S and MM.1 S BCMA knockout by UTD and TriPRIL CAR T cells.

FIG. 29D shows the killing of MM.1S and MM.1 S BCMA knockout cells by BCMA CAR and TriPRIL CAR T cells in vitro.

FIG. 29E shows kinetics of tumor growth over time of MM.1S and MM.1 S BCMA-negative cell lines in NSG mice.

FIGS. 30A-30C show the anti-tumor efficacy of the CAR T cells assessed in a xenograft model of BCMA-negative multiple myeloma. FIG. 30A shows the experimental design. FIG. 30B shows representative bioluminescence imaging of myeloma xenografts over time. FIG. 30C shows the quantification of flux (photons/second) in the three groups at the indicated time points.

FIG. 31A shows polyfunctionality of CD4+ or CD8+ T cells with various CAR constructs averaged from three donors. FIG. 31B shows polyfunctionality of CD4+ or CD8+ T cells with various CAR constructs from each individual donor.

FIG. 32A shows polyfunctional strength index (PSI) of CD+ or CD8+ T cells with various CAR constructs averaged from three donors. FIG. 32B shows PSI of CD+ or CD8+ T cells with various CAR constructs from each individual donor.

FIG. 33A shows polyfunctional heatmaps of CD4+ or CD8+ T cells averaged from three donors.

FIG. 33B shows polyfunctional heatmaps of CD4+ or CD8+ T cells from each individual donor.

FIG. 34A shows protein secretions (%) of CD4+ or CD8+ T cells with various CAR constructs averaged from three donors. FIGS. 34B-34D show protein secretions (%) of CD4+ or CD8+ T cells with various CAR constructs from each individual donor.

FIG. 35 shows cytotoxicity of TriPRIL CARTs in the presence of recombinant human (rh) BCMA, rhTACI and rhAPRIL.

### DETAILED DESCRIPTION OF THE INVENTION

Described herein are improvements in CAR design that avoid off-target effects and reduce the possibility for tumor escape by loss of target antigen. In one embodiment, an approach is described herein that uses a single ligand that binds two different tumor-related antigens or factors. The single ligand is fused to transmembrane and T cell receptor intracellular effector domains, optionally with co-stimulatory domains, essentially as for CARs known in the art. A CAR with a ligand that binds two different tumor-associated antigens or factors will not lose effectiveness if one or the other of the antigens or factors is down-regulated by targeted cells. In some embodiments, the CAR includes a ligand that includes a portion of a TNF superfamily receptor ligand. This is illustrated herein using as a ligand a portion of the APRIL polypeptide, which binds with high affinity to both the multiple myeloma and leukemia-associated BCMA polypeptide and TACI, another factor expressed on multiple myelomas.

Embodiments of the technology described herein relate to the discovery that a T cell comprising a CAR polypeptide comprising an extracellular portion of a TNF superfamily receptor ligand (e.g., APRIL) is an efficient therapeutic to treat cancer, a plasma cell disorder, or an autoimmune disease, without invoking off-target effects or adverse reactions.

Accordingly, one aspect of the invention described herein relates to a CAR polypeptide comprising a) an extracellular domain comprising a portion of a TNF superfamily receptor ligand (e.g., APRIL), which is N-terminal to the endogenous cleavage site, and comprises at least the TNF-like domain, b) a hinge and transmembrane domain, and c) an intracellular signaling domain. In some embodiments, the TNF superfamily receptor ligand is APRIL. In other embodiments, the TNF superfamily receptor ligand is TNF-alpha, lymphotoxin beta, OX40L, CD154, FasL, LIGHT, TL1A, CD70, Siva, CD153, 4-1BB ligand, TRAIL, RANKL, TWEAK, BAFF, CAMLG, LIGHT, NGF, BDNF, NT-3, NT-4, GITR ligand, TL1A, or EDA-A2.

Furthermore, the invention described herein relates to CAR polypeptides comprising two or more (e.g., three) extracellular domains, each comprising a portion of a TNF superfamily receptor ligand (e.g., APRIL) as described herein, as well as related methods of their use for treating a disorder (e.g., a cancer (e.g., multiple myeloma), a plasma cell disorder, autoimmune disease or disorder, or transplant rejection) in a subject resistant to anti-BCMA therapy.

Considerations necessary to make and use these and other aspects of the technology are described in the following.

Chimeric Antigen Receptors

The technology described herein provides improved chimeric antigen receptors (CARs) for use in immunotherapy. The following discusses CARs and the various improvements.

The terms “chimeric antigen receptor” or “CAR” or “CARs” as used herein refer to engineered T cell receptors, which graft a ligand or antigen specificity onto, e.g., T cells (for example naïve T cells, central memory T cells, effector memory T cells or combinations thereof). CARs are also known as artificial T-cell receptors, chimeric T-cell receptors or chimeric immunoreceptors.

A CAR places a chimeric extracellular target-binding domain that specifically binds a target, e.g., a polypeptide expressed on the surface of a cell to be targeted for an immune, e.g., a T cell response onto a construct including a transmembrane domain, and intracellular domain(s) (including signaling domains) of a T cell receptor molecule. In one embodiment, the chimeric extracellular target-binding domain comprises the antigen-binding domain(s) of an antibody that specifically binds an antigen expressed on a cell to be targeted for an immune, e.g., a T cell response. The properties of the intracellular signaling domain(s) of the CAR can vary as known in the art and as disclosed herein, but the chimeric target/antigen-binding domains(s) render the receptor sensitive to signaling activation when the chimeric target/antigen binding domain binds the target/antigen on the surface of a targeted cell.

With respect to intracellular signaling domains, so-called “first-generation” CARs include those that solely provide, e.g., CD3zeta (CD3) signals upon antigen binding. So-called “second-generation” CARs include those that provide both co-stimulation (e.g., CD28 or CD137) and activation (CD3) domains, and so-called “third-generation” CARs include those that provide multiple costimulatory (e.g., CD28 and CD137) domains and activation domains (e.g., CD3). In various embodiments, the CAR is selected to have high affinity or avidity for the target/antigen—for example, antibody-derived target or antigen binding domains will generally have higher affinity and/or avidity for the target antigen than would a naturally-occurring T cell receptor. This property, combined with the high specificity one can select for an antibody provides highly specific T cell targeting by CAR T cells.

As used herein, a “CAR T cell” or “CAR-T” refers to a T cell which expresses a CAR. When expressed in a T cell, CARs have the ability to redirect T-cell specificity and reactivity toward a selected target in a non-MHC-restricted manner, exploiting the antigen-binding properties of monoclonal antibodies. The non-MHC-restricted antigen recognition gives T-cells expressing CARs the ability to recognize an antigen independent of antigen processing, thus bypassing a major mechanism of tumor escape.

As used herein, the term “extracellular target binding domain” refers to a polypeptide found on the outside of the cell sufficient to facilitate binding to a target. The extracellular target binding domain will specifically bind to its binding partner. As non-limiting examples, the extracellular target-binding domain can include an antigen-binding domain of an antibody, or a ligand (e.g., a TNF superfamily receptor ligand). In some embodiments, the extracellular target-binding domain comprises APRIL, or two or more APRIL domains, for example, three APRIL domains (TriPRIL) as described herein), which recognizes and binds with a cognate binding partner protein. In this context, a ligand is a molecule which binds specifically to a portion of a protein and/or receptor. The cognate binding partner of a ligand useful in the methods and compositions described herein can generally be found on the surface of a cell. Ligand:cognate partner binding can result in the alteration of the ligand-bearing receptor, or activate a physiological response, for example, the activation of a signaling pathway or cascade. In one embodiment, the ligand can be non-native to the genome. Optionally, the ligand has a conserved function across at least two species.

Antibody Reagents

In various embodiments, the CARs described herein comprise an antibody reagent or an antigen-binding domain thereof as an extracellular target-binding domain.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like. Fully human antibody binding domains can be selected, for example, from phage display libraries using methods known to those of ordinary skill in the art.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

In one embodiment, the antibody or antibody reagent is not a human antibody or antibody reagent, (i.e., the antibody or antibody reagent is mouse), but has been humanized. A “humanized antibody or antibody reagent” refers to a non-human antibody or antibody reagent that has been modified at the protein sequence level to increase its similarity to antibody or antibody reagent variants produced naturally in humans. One approach to humanizing antibodies employs the grafting of murine or other non-human CDRs onto human antibody frameworks.

In one embodiment, a CAR's extracellular target binding domain comprises or consists essentially of a single-chain Fv (scFv) fragment created by fusing the VH and VL domains of an antibody, generally a monoclonal antibody, via a flexible linker peptide. In various embodiments, the scFv is fused to a transmembrane domain and to a T cell receptor intracellular signaling domain, e.g., an engineered intracellular signaling domain as described herein.

Antibody binding domains and ways to select and clone them are well known to those of ordinary skill in the art.

In one embodiment, the CARs useful in the technology described herein comprise at least two antigen-specific targeting regions in an extracellular target binding domain, a transmembrane domain, and an intracellular signaling domain. In such embodiments, the two or more antigen-specific targeting regions target at least two different antigens and may be arranged in tandem and separated by linker sequences. In another embodiment, the CAR is a bispecific CAR. A bispecific CAR is specific to two different antigens.

TNF Superfamily Receptor Ligands

In one embodiment, the extracellular domain of the CAR polypeptide comprises a portion of a TNF superfamily receptor ligand, wherein the portion of the TNF superfamily receptor ligand is N-terminal to the endogenous cleavage site, and comprises at least the TNF-like domain.

For example, in one embodiment, the extracellular domain of the CAR polypeptide comprises a portion of APRIL, wherein the portion of APRIL is N-terminal to the endogenous cleavage site, and comprises at least the TNF-like domain (SEQ ID NO: 37).

(SEQ ID NO: 37)
VLHLVPINATSKDDSDVTEVMWQPALRRGRGLQAQGYGVRIQDAGVYLLY
SQVLFQDVTFTMGQVVSREGQGRQETLFRCIRSMPSHPDRAYNSCYSAGV
FHLHQGDILSVIIPRARAKLNLSPHGTFLGFV

In other embodiments, the CAR polypeptide comprises a TNF superfamily receptor ligand or portion thereof, wherein the TNF superfamily receptor ligand is TNF-alpha, lymphotoxin beta, OX40 ligand (OX40L), CD154, Fas ligand (FasL), LIGHT, TNF-like ligand 1A (TL1A), CD70, Siva, CD153, 4-1 BB ligand (4-1 BBL), TNF-related apoptosis-inducing ligand (TRAIL), receptor activator of nuclear factor kappa-B ligand (RANKL), TNF-related weak inducer of apoptosis (TWEAK), B cell activating factor (BAFF), calcium modulating ligand (CAMLG or CAML), nerve growth factor (NGF), brain-derived neurotrophic factor (BDNF), neurotrophin-3 (NT-3), neurotrophin-4 (NT-4), glucocorticoid-induced TNFR-related protein (GITR) ligand, or ectodysplasin A2 (EDA-A2). In some embodiments, the TNF superfamily receptor ligand or portion thereof, e.g., TNF-alpha, lymphotoxin beta, OX40L, CD154, FasL, LIGHT, TL1A, CD70, Siva, CD153, 4-1BBL, TRAIL, RANKL, TWEAK, BAFF, CAMLG, NGF, BDNF, NT-3, NT-4, GITR ligand, or EDA-A2, includes the TNF-like domain of SEQ ID NO: 37.

In some embodiments, a CAR polypeptide described herein can comprise two or more (e.g., two or more, three or more, four or more, five or more, six or more, seven or more, eight or more, nine or more, or ten or more) extracellular domains, each comprising a portion of a TNF superfamily receptor ligand. The extracellular target binding domain of a CAR polypeptide described herein can include two or more of the same TNF superfamily receptor ligand or portion thereof (e.g., a homooligomer), or may include at least one different TNF superfamily receptor ligand or portion thereof (e.g., a heterooligomer).

TriPRIL

In some embodiments, the extracellular target-binding domain of the CAR comprises a ligand of BCMA and/or TACI, e.g., APRIL, or a portion thereof. The extracellular target-binding domain can comprise two or more APRIL domains. In one embodiment, the extracellular target-binding domain of the CAR comprises three APRIL domains, which are optionally connected by one or more linker sequences. Exemplary linkers described herein include glycine/serine linkers, as well as a Whitlow linker. Such a polypeptide comprising three APRIL domains is referred to herein as TriPRIL.

APRIL (A Proliferation-Inducing Ligand), also known as TNF Superfamily Member 13 (TNFSF13), is a member of the tumor necrosis factor ligand (TNF) family, and functions as a ligand for BCMA. APRIL sequences are known for a number of species, e.g., human APRIL (UniProtKB: 075888; NCBI Gene ID: 8741) polypeptide (e.g., NCBI Ref Seq: NP 001185551.1) and mRNA (e.g., NCBI Ref Seq: NM_001198622.1). APRIL can refer to human APRIL (e.g., SEQ ID NO: 60), including naturally occurring variants, truncated forms (e.g. SEQ ID NO: 21, 27, 33, or 40), molecules, and alleles thereof. In some embodiments of any of the aspects, e.g., in veterinary applications, APRIL can refer to the APRIL of, e.g., dog, cat, cow, horse, pig, and the like. Homologs and/or orthologs of human APRIL are readily identified for such species by one of skill in the art, e.g., using the NCBI ortholog search function or searching available sequence data for a given species for sequence similar to a reference APRIL sequence. In some embodiments, human APRIL corresponds to the sequence of SEQ ID NO: 60:

(SEQ ID NO: 60)
MPASSPFLLAPKGPPGNMGGPVREPALSVALWLSWGAALGAVACAMALLT
QQTELQSLRREVSRLQGTGGPSQNGEGYPWQSLPEQSSDALEAWENGERS
RKRRAVLTQKQKKQHSVLHLVPINATSKDDSDVTEVMWQPALRRGRGLQA
QGYGVRIQDAGVYLLYSQVLFQDVTFTMGQVVSREGQGRQETLFRCIRSM
PSHPDRAYNSCYSAGVFHLHQGDILSVIIPRARAKLNLSPHGTFLGFVKL

In one embodiment, the portion of APRIL has a sequence corresponding to a sequence selected from SEQ ID NO: 3, 8, 9, 15, 21, 27, 33, or 40; or comprises a sequence selected from SEQ ID NO: 3, 8, 9, 15, 21, 27, 33, or 40; or comprises a sequence with at least 80%, at least 85%, at least 90%, at least 91%, at least 92%, at least 93%, at least 94%, at least 95%, at least 96%, at least 97%, at least 98%, at least 99%, or at least 100% sequence identity to a sequence selected from SEQ ID NO 3, 8, 9, 15, 21, 27, 33, or 40. In one embodiment, the portion of APRIL consists essentially of a sequence selected from SEQ ID NO: 3, 8, 9, 15, 21, 27, 33, or 40; or consists essentially of a sequence with at least 80%, at least 85%, at least 90%, at least 91%, at least 92%, at least 93%, at least 94%, at least 95%, at least 96%, at least 97%, at least 98%, at least 99%, or at least 100% sequence identity to a sequence selected from SEQ ID NO 3, 8, 9, 15, 21, 27, 33, or 40. In one embodiment, the portion of APRIL does not comprise a sequence derived from the portion of APRIL which is C-terminal of the endogenous cleavage site.

Linker sequences useful for the invention can be, for example, from 2 to 100 amino acids, 5 to 50 amino acids, 10 to 15 amino acids, 15 to 20 amino acids, or 18 to 20 amino acids in length, and include any suitable linkers known in the art. For instance, linker sequences useful for the invention include, but are not limited to, glycine/serine linkers, e.g., GGGSGGGSGGGS (SEQ ID NO: 41) and Gly4Ser (G4S) linkers such as (G4S)3 (GGGGSGGGGSGGGGS (SEQ ID NO: 61)) and (G4S)4 (GGGGSGGGGSGGGGSGGGGS (SEQ ID NO: 62)); the linker sequence of GSTSGSGKPGSGEGSTKG (SEQ ID NO: 58) as described by Whitlow et al., Protein Eng. 6(8):989-95, 1993, the contents of which are incorporated herein by reference in its entirety; the linker sequence of GGSSRSSSSGGGGSGGGG (SEQ ID NO: 63) as described by Andris-Widhopf et al., Cold Spring Harb. Protoc. 2011(9), 2011, the contents of which are incorporated herein by reference in its entirety; as well as linker sequences with added functionalities, e.g., an epitope tag or an encoding sequence containing Cre-Lox recombination site as described by Sblattero et al., Nat. Biotechnol. 18(1):75-80, 2000, the contents of which are incorporated herein by reference in its entirety.

For example, a linker sequence connecting two APRIL domains can correspond to the sequence of SEQ ID NO: 41, 58, 61, 62, or 63, or comprise the sequence of SEQ ID NO: 41, 58, 61, 62, or 63, or comprise a sequence having at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 41, 58, 61, 62, or 63, or comprises a sequence having at least 1, 2, 3, 4, 5, 6, or 7 amino acid substitution(s) or deletion(s) relative to the sequence of SEQ ID NO: 41, 58, 61, 62, or 63.

In one example, TriPRIL includes glycine/serine linkers between each APRIL domain. An exemplary TriPRIL sequence having glycine/serine linkers is as follows:

(SEQ ID NO: 56)
HSVLHLVPINATSKDDSDVTEVMWQPALRRGRGLQAQGYGVRIQDAGVYL
LYSQVLFQDVTFTMGQVVSREGQGRQETLFRCIRSMPSHPDRAYNSCYSA
GVFHLHQGDILSVIIPRARAKLNLSPHGTFLGFVKLGGGSGGGSGGGSHS
VLHLVPINATSKDDSDVTEVMWQPALRRGRGLQAQGYGVRIQDAGVYLLY
SQVLFQDVTFTMGQVVSREGQGRQETLFRCIRSMPSHPDRAYNSCYSAGV
FHLHQGDILSVIIPRARAKLNLSPHGTFLGFVKLGGGSGGGSGGGSHSVL
HLVPINATSKDDSDVTEVMWQPALRRGRGLQAQGYGVRIQDAGVYLLYSQ
VLFQDVTFTMGQVVSREGQGRQETLFRCIRSMPSHPDRAYNSCYSAGVFH
LHQGDILSVIIPRARAKLNLSPHGTFLGFVKL,

wherein the linker sequences are presented in bold and underlined. In some embodiments, the amino acid sequence of TriPRIL corresponds to the sequence of SEQ ID NO: 56, comprises the amino acid sequence of SEQ ID NO: 56, or comprises an amino acid sequence having at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 56.

In a further example, TriPRIL can include three APRIL domains connected by a Whitlow linker sequence as follows:

(SEQ ID NO: 59)
HSVLHLVPINATSKDDSDVTEVMWQPALRRGRGLQAQGYGVRIQDAGVYL
LYSQVLFQDVTFTMGQVVSREGQGRQETLFRCIRSMPSHPDRAYNSCYSA
GVFHLHQGDILSVIIPRARAKLNLSPHGTFLGFVKLGSTSGSGKPGSGEG
STKGHSVLHLVPINATSKDDSDVTEVMWQPALRRGRGLQAQGYGVRIQDA
GVYLLYSQVLFQDVTFTMGQVVSREGQGRQETLFRCIRSMPSHPDRAYNS
CYSAGVFHLHQGDILSVIIPRARAKLNLSPHGTFLGFVKLGSTSGSGKPG
SGEGSTKGHSVLHLVPINATSKDDSDVTEVMWQPALRRGRGLQAQGYGVR
IQDAGVYLLYSQVLFQDVTFTMGQVVSREGQGRQETLFRCIRSMPSHPDR
AYNSCYSAGVFHLHQGDILSVIIPRARAKLNLSPHGTFLGFVKL,

wherein the linker sequences are presented in bold and underlined. In some embodiments, the amino acid sequence of TriPRIL corresponds to the sequence of SEQ ID NO: 59, comprises the amino acid sequence of SEQ ID NO: 59, or comprises an amino acid sequence having at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 59.

In some embodiments, each APRIL domain of TriPRIL corresponds to the sequence of SEQ ID NO: 21, 27, 33, 40, or 60; or comprises the sequence of SEQ ID NO: 21, 27, 33, 40, or 60; or comprises a sequence having at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 21, 27, 33, 40, or 60. In one embodiment, the portion of APRIL consists essentially of the sequence of SEQ ID NO: 21, 27, 33, or 40; or consists essentially of a sequence having at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 21, 27, 33, or 40. In one embodiment, each APRIL domain of TriPRIL does not comprise a sequence derived from the portion of APRIL that is C-terminal of the endogenous cleavage site.

In one embodiment, the CAR polypeptide comprises a portion of a TNF superfamily receptor ligand that comprises one or more mutations within its coding region. For example, in one embodiment, the CAR polypeptide comprises a portion of APRIL that comprises one or more mutations within its coding region. Exemplary amino acid mutations include point mutation made to amino acids 18, 61, 91, 92, and/or 117 of SEQ ID NO: 21; amino acids 18, 63, 91, 92, and/or 117 of SEQ ID NO: 27; amino acids 18, 63, 91, 92, and/or 117 of SEQ ID NO: 33; amino acids 18, 61, 91, 92, and/or 117 of SEQ ID NO: 40; and amino acids 18, 63, 91, 92, and/or 117 of SEQ ID NO: 40. In another example, the portion of APRIL can include an amino acid substitution or deletion at amino acids 18, 61, 91, 92, and/or 117 of SEQ ID NO: 21; amino acids 18, 63, 91, 92, and/or 117 of SEQ ID NO: 27; amino acids 18, 63, 91, 92, and/or 117 of SEQ ID NO: 33; amino acids 18, 61, 91, 92, and/or 117 of SEQ ID NO: 40; and amino acids 18, 63, 91, 92, and/or 117 of SEQ ID NO: 40. One skilled in the art will be capable of introducing mutations into the nucleic acid sequence of a gene or gene product using standard techniques. For example, point mutations can be introduced via site-directed point mutagenesis, a PCR technique. Site-directed mutagenesis kits are commercially available, for instance, through New England Biolabs; Ipswich, Mass. Non-limiting examples of alternative methods to introduce point mutations to the nucleic acid sequence of a gene or gene product include cassette mutagenesis or whole plasmid mutagenesis.

Optionally, the portion of a TNF superfamily receptor ligand (e.g., APRIL) does not comprise a lysine-rich region. In one embodiment, a “lysine-rich region” refers to a region of the amino acid sequence that comprises at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% lysine amino acids. As used herein a “region” refers to at least 4 or more consecutive amino acids. In one embodiment, the lysine rich sequence comprises a sequence of KQKKQH (SEQ ID NO: 38).

Further, in another example, a CAR polypeptide comprising a portion of APRIL does not comprise a furin cleavage site of R—X—Y—R (SEQ ID NO: 66), wherein X is any amino acid and Y is lysine or arginine. For example, the portion of APRIL does not comprise a furin cleavage site having the sequence of R—K—R—R (SEQ ID NO: 67). A furin cleavage site of human APRIL as described herein may be deleted or mutated.

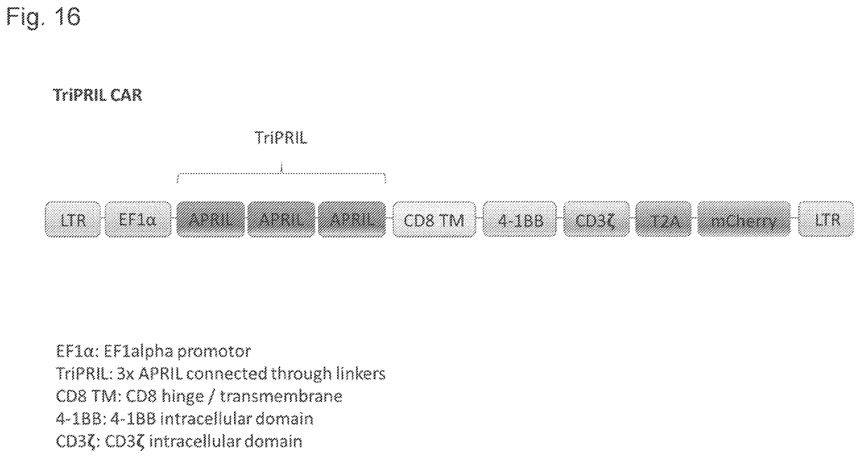

In one embodiment of any aspect, the CAR polypeptide comprises two or more (e.g., two or more, three or more, four or more, five or more, six or more, seven or more, eight or more, nine or more, or ten or more) extracellular domains comprising a portion of a TNF superfamily receptor ligand (e.g., APRIL). In one embodiment, the CAR polypeptide comprises three extracellular domains comprising a portion of TNF superfamily receptor ligand (e.g., APRIL). For example, the CAR polypeptide comprises three extracellular domains each comprising a portion of APRIL, i.e., TriPRIL as shown in FIG. 16. For example, in some embodiments, the CAR polypeptide may include a repeat of two or more (e.g., two or more, three or more, four or more, five or more, six or more, seven or more, eight or more, nine or more, or ten or more) TNF superfamily receptor ligands (e.g., APRIL, as is shown in FIG. 16 where APRIL is provided as a triple repeat). In some embodiments, the TNF superfamily receptor ligands are the same. In other embodiments, the TNF superfamily receptor ligands may be different (e.g., the CAR may include one or more portions of APRIL and one or more portions of a second TNF superfamily receptor ligand (e.g., BAFF)).

In one embodiment of any aspect, the TNF superfamily receptor ligand (e.g., APRIL) oligomerizes (e.g., dimerizes or trimerizes) with another TNF superfamily receptor ligand (e.g., APRIL). The oligomerization may be intramolecular or intermolecular. The oligomer may be a homooligomer or a heterooligomer.

Target/Antigen

Any cell-surface moiety can be targeted by a CAR. Most often, the target will be a cell-surface polypeptide differentially or preferentially expressed on a cell one wishes to target for a T cell response. In this regard, tumor antigens or tumor-associated antigens provide attractive targets, providing a means to target tumor cells while avoiding or at least limiting collateral damage to non-tumor cells or tissues. Non-limiting examples of tumor antigens or tumor-associated antigens include CEA, Immature laminin receptor, TAG-72, HPV E6 and E7, BING-4, Calcium-activated chloride channel 2, Cyclin B1, 9D7, Ep-CAM, EphA3, Her2/neu, Telomerase, Mesotheliun, SAP-1, Survivin, BAGE family, CAGE family, GAGE family, MAGE family, SAGE family, XAGE family, NY-ESO-1/LAGE-1, PRAME, SSX-2, Melan-A/MART-1, Gp100/pme117, Tyrosinase, TRP-1/-2, MC1R, BRCA1/2, CDK4, MART-2, p53, Ras, MUC1, and TGF-(3R11. In one aspect, the cell-surface moiety may be a TNF superfamily receptor, e.g., TNFR1, TNFR2, CD95, DCR3, DR3, DR4, DR5, DCR1, DCR2, DR6, EDAR, NGFR, OPG, RANK, LTbetaR, FN14, HVEM, CD27, CD3θ, CD40, 4-1BB, OX40, GITR, BCMA, TACI, BAFFR, XEDAR, TROY, or RELT. In some embodiments, the TNF superfamily receptor is BCMA or TACI.

In some embodiments, the target/antigen of a CAR polypeptide as described herein is BCMA and/or TACI.

B cell maturation antigen (BCMA) is a small type-III transmembrane protein and is a member of the tumor necrosis factor (TNF) receptor superfamily. BCMA binds BAFF with low affinity and APRIL with high affinity, and BCMA signaling protects myeloma cells from apoptosis. BCMA has two close family members: TACI and BAFF receptor.

BCMA sequences are known for a number of species, e.g., human BCMA (NCBI Gene ID: 608) polypeptide (e.g., NCBI Ref Seq: NP_001183.2) and mRNA (e.g., NCBI Ref Seq: NM_001192.2). BCMA can refer to human BCMA, including naturally occurring variants, molecules, and alleles thereof. In some embodiments of any of the aspects, e.g., in veterinary applications, BCMA can refer to the BCMA of, e.g., dog, cat, cow, horse, pig, and the like. Homologs and/or orthologs of human BCMA are readily identified for such species by one of skill in the art, e.g., using the NCBI ortholog search function or searching available sequence data for a given species for sequence similar to a reference BCMA sequence.

Transmembrane activator and CAML interactor (TACI) is receptor that recognizes APRIL, BAFF, and CAML. TACI is a member of the TNF receptor superfamily and is expressed at similar levels and stages of B cell development as BCMA. The intracellular domains of both BCMA and TACI interact with TRAFs, and likely have redundant functions in promoting plasma cell survival. TACI sequences are known for a number of species, e.g., human TACI (NCBI Gene ID: 23495) polypeptide (e.g., NCBI Ref Seq: NP_036584.1) and mRNA (e.g., NCBI Ref Seq: NM_012452.2). TACI can refer to human TACI, including naturally occurring variants, molecules, and alleles thereof. In some embodiments of any of the aspects, e.g., in veterinary applications, TACI can refer to the TACI of, e.g., dog, cat, cow, horse, pig, and the like. Homologs and/or orthologs of human TACI are readily identified for such species by one of skill in the art, e.g., using the NCBI ortholog search function or searching available sequence data for a given species for sequence similar to a reference TACI sequence.

In some embodiments, APRIL, or a portion thereof, binds to TACI via residue 206 of full-length APRIL (SEQ ID NO: 60). In some embodiments, APRIL, or a portion hereof, binds to BCMA via residue 132 of full-length APRIL (SEQ ID NO: 60). Information regarding the binding of APRIL to its receptors BCMA and TACI is described in detail in Kimberley et al., The Journal of Biological Chemistry. 287(44):37434-37446, 2012, which is incorporated herein by reference in its entirety.

Hinge and Transmembrane Domains

Each CAR as described herein necessarily includes a transmembrane domain that joins the extracellular target-binding domain to the intracellular signaling domain.

As used herein, “hinge domain” refers to an amino acid region that allows for separation and flexibility of the binding moiety and the T cell membrane. The length of the flexible hinges also allow for better binding to relatively inaccessible epitopes, e.g., longer hinge regions are allow for optimal binding. One skilled in the art will be able to determine the appropriate hinge for the given CAR target. In one embodiment, the transmembrane domain or fragment thereof of any of the CAR polypeptides described herein comprises a CD8 or 4-1 BB hinge domain.

Each CAR as described herein necessarily includes a transmembrane domain that joins the extracellular target-binding domain to the intracellular signaling domain.

Accordingly, in some embodiments, the binding domain of the CAR is optionally followed by one or more “hinge domains,” which plays a role in positioning the target binding domain away from the effector cell surface to enable proper cell/cell contact, target binding and activation. A CAR optionally comprises one or more hinge domains between the binding domain and the transmembrane domain (TM). The hinge domain may be derived either from a natural, synthetic, semi-synthetic, or recombinant source. The hinge domain can include the amino acid sequence of a naturally occurring immunoglobulin hinge region or an altered immunoglobulin hinge region. Illustrative hinge domains suitable for use in the CARs described herein include the hinge region derived from the extracellular regions of type 1 membrane proteins such as CD8 (e.g., CD8a), CD4, CD28, 4-1 BB, and CD7, which may be wild-type hinge regions from these molecules or may be altered. In some embodiments, the hinge region is derived from the hinge region of an immunoglobulin-like protein (e.g., IgA, IgD, IgE, IgG, or IgM), CD28, or CD8. In one embodiment, the hinge domain comprises a CD8a hinge region.

As used herein, “transmembrane domain” (TM domain) refers to the generally hydrophobic region of the CAR which crosses the plasma membrane of a cell. The TM domain can be the transmembrane region or fragment thereof of a transmembrane protein (for example a Type I transmembrane protein or other transmembrane protein), an artificial hydrophobic sequence, or a combination thereof. While specific examples are provided herein and used in the Examples, other transmembrane domains will be apparent to those of skill in the art and can be used in connection with alternate embodiments of the technology. A selected transmembrane region or fragment thereof would preferably not interfere with the intended function of the CAR. As used in relation to a transmembrane domain of a protein or polypeptide, “fragment thereof” refers to a portion of a transmembrane domain that is sufficient to anchor or attach a protein to a cell surface.

In one embodiment, the transmembrane domain or fragment thereof of any of the CAR polypeptides described herein comprises a transmembrane domain selected from the transmembrane domain of CD8 or 4-1 BB. In an alternate embodiment of any aspect, the transmembrane domain or fragment thereof of the CAR described herein comprises a transmembrane domain selected from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1 BB (CD137), GITR, CD40, BAFFR, HVEM (LIGHTR), SLAMF7, NKp80 (KLRFI), CD160, CD19, IL2R beta, IL2R gamma, IL7Ra, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11 b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

4-1 BB is a membrane receptor protein, also known as CD137, and is a member of the tumor necrosis factor (TNF) receptor superfamily. 4-1 BB is expressed on activated T lymphocytes. 4-1 BB sequences are known for a number of species, e.g., human 4-1 BB, also known as TNFRSF9 (NCBI Gene ID: 3604) and mRNA (NCBI Reference Sequence: NM_001561.5). 4-1 BB can refer to human 4-1 BB, including naturally occurring variants, molecules, and alleles thereof. In some embodiments of any of the aspects, e.g., in veterinary applications, 4-1 BB can refer to the 4-1 BB of, e.g., dog, cat, cow, horse, pig, and the like. Homologs and/or orthologs of human 4-1 BB are readily identified for such species by one of skill in the art, e.g., using the NCBI ortholog search function or searching available sequence data for a given species for sequence similar to a reference 4-1 BB sequence.

As used herein, a hinge and transmembrane domain or a hinge/transmembrane domain refers to a domain comprising both a hinge and a transmembrane domain. In one embodiment, the 4-1 BB hinge and transmembrane sequence corresponds to a nucleotide sequence selected from SEQ ID NO: 10 or 16; or comprises a sequence selected from SEQ ID NO: 10 or 16; or comprises a sequence with at least 80%, at least 85%, at least 90%, at least 91%, at least 92%, at least 93%, at least 94%, at least 95%, at least 96%, at least 97%, at least 98%, at least 99%, or at least 100% sequence identity to a sequence selected from SEQ ID NO: 10 or 16. In one embodiment, the 4-1 BB hinge and transmembrane sequence corresponds to an amino acid sequence selected from SEQ ID NO: 28 or 34; or comprises a sequence selected from SEQ ID NO: 28 or 34; or comprises a sequence with at least 80%, at least 85%, at least 90%, at least 91%, at least 92%, at least 93%, at least 94%, at least 95%, at least 96%, at least 97%, at least 98%, at least 99%, or at least 100% sequence identity to a sequence selected from SEQ ID NO: 28 or 34.

CD8 is an antigen preferentially found on the cell surface of cytotoxic T lymphocytes. CD8 mediates cell-cell interactions within the immune system, and acts as a T cell co-receptor. CD8 consists of an alpha (CD8a or CD8a) and beta (CD8(3 or CD8b) chain. CD8a sequences are known for a number of species, e.g., human CD8a, (NCBI Gene ID: 925) polypeptide (NCBI Ref Seq NP_001139345.1) and mRNA (e.g., NCBI Ref Seq NM_000002.12). CD8 can refer to human CD8, including naturally occurring variants, molecules, and alleles thereof. In some embodiments of any of the aspects, e.g., in veterinary applications, CD8 can refer to the CD8 of, e.g., dog, cat, cow, horse, pig, and the like. Homologs and/or orthologs of human CD8 are readily identified for such species by one of skill in the art, e.g., using the NCBI ortholog search function or searching available sequence data for a given species for sequence similar to a reference CD8 sequence.

In one embodiment, the CD8 hinge and transmembrane domain sequence corresponds to the nucleotide sequence of SEQ ID NO: 4 or 53; or comprises the sequence of SEQ ID NO: 4 or 53; or comprises a sequence with at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 4 or 53. In one embodiment, the CD8 hinge and transmembrane sequence corresponds to the amino acid sequence of SEQ ID NO: 22 or 42; or comprises the sequence of SEQ ID NO: 22 or 42; or comprises a sequence with at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 22 or 42.

Co-Stimulatory Domain

Each CAR described herein optionally comprises an intracellular domain of a co-stimulatory molecule, or co-stimulatory domain. As used herein, the term “co-stimulatory domain” refers to an intracellular signaling domain of a co-stimulatory molecule. Co-stimulatory molecules are cell surface molecules other than antigen receptors or Fc receptors that provide a second signal required for efficient activation and function of T lymphocytes upon binding to antigen. Illustrative examples of such co-stimulatory molecules include CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAGS), CD270 (HVEM), CD273 (PD-L2), CD274 (PD-L1), CD278 (ICOS), DAP10, LAT, NKD2C SLP76, TRIM, and ZAP70. In one embodiment, the intracellular domain is the intracellular domain of 4-1 BB, CD27, CD28, or OX40.

In one embodiment, the CAR polypeptide further comprises an intracellular domain. As used herein, an “intracellular domain” refers to a nucleic acid fully comprised within a cell. In one embodiment, the intracellular domain refers to the intracellular domain of a receptor. An intracellular domain can interact with the interior of a cell. With respect to the intracellular domain of a receptor, the intracellular domain can function to relay a signal transduced. An intracellular domain of a receptor can comprise enzymatic activity.

In one embodiment, the intracellular domain is the intracellular domain of a 4-1 BB. In one embodiment, the 4-1 BB intracellular domain sequence corresponds to a nucleotide sequence selected from SEQ ID NO: 5, 11, 17, or 54; or comprises a sequence selected from SEQ ID NO: 5, 11, 17, or 54; or comprises at least 80%, at least 85%, at least 90%, at least 95%, at least 96%, at least 97%, at least 98%, at least 99%, or at least 100% sequence identity to a sequence selected from SEQ ID NO: 5, 11, 17, or 54. In one embodiment, the 4-1 BB intracellular domain amino acid sequence corresponds to an amino acid sequence selected from SEQ ID NO: 23, 29, 35, or 43; or comprises a sequence selected from SEQ ID NO: 23, 29, 35, or 43; or comprises at least 80%, at least 85%, at least 90%, at least 95%, at least 96%, at least 97%, at least 98%, at least 99%, or at least 100% sequence identity to a sequence selected from SEQ ID NO: 23, 29, 35, or 43.

Intracellular Signaling Domain

CARs as described herein comprise an intracellular signaling domain. An “intracellular signaling domain,” refers to the part of a CAR polypeptide that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited following antigen binding to the extracellular target binding domain of the CAR.

CD3 is a T cell co-receptor that facilitates T lymphocytes activation when simultaneously engaged with the appropriate co-stimulation (e.g., binding of a co-stimulatory molecule). A CD3 complex consists of 4 distinct chains; mammal CD3 consists of a CD3γ chain, a CD3δ chain, and two CD3ε chains. These chains associate with a molecule known as the T cell receptor (TCR) and the CD3ζ to generate an activation signal in T lymphocytes. A complete TCR complex comprises a TCR, CD3ζ, and the complete CD3 complex.

In some embodiments of any aspect, a CAR polypeptide described herein comprises an intracellular signaling domain that comprises an Immunoreceptor Tyrosine-based Activation Motif or ITAM from CD3 zeta (CD3ζ), ITAM-mutated CD3ζ, CD3η, or CD3θ. In some embodiments of any aspect, the ITAM comprises three motifs of ITAM of CD3ζ (ITAM3). In some embodiments of any aspect, the three motifs of ITAM of CD3ζ are mutated.

ITAMS are known as a primary signaling domains regulate primary activation of the TCR complex either in a stimulatory way, or in an inhibitory way. Primary signaling domains that act in a stimulatory manner may contain signaling motifs which are known as immunoreceptor tyrosine-based activation motifs or ITAMs. Non-limiting examples of ITAM containing intracellular signaling domains that are of particular use in the technology include those derived from TCFζ, FcRγ, FcRβ, CD3γ, CD3θ, CD3δ, CD3η, CD3ε, CD3ζ, CD22, CD79a, CD79b, and CD66d.

One skilled in the art will be capable of introducing mutations into the nucleic acid sequence of a gene or gene product, for example ITAM, using standard techniques. For example, point mutations can be introduced via site-directed point mutagenesis, a PCR technique. Site-directed mutagenesis kits are commercially available, for instance, through New England Biolabs; Ipswich, Mass. Non-limiting examples of alternative methods to introduce point mutations to the nucleic acid sequence of a gene or gene product include cassette mutagenesis or whole plasmid mutagenesis.

In one embodiment, the ITAM utilized in the CAR is based on alternatives to CD3ζ, including mutated ITAMs from CD3ζ (which contains 3 ITAM motifs), truncations of CD3ζ, and alternative splice variants known as CD3ε, CD3η, CD3θ, and artificial constructs engineered to express fusions between CD3ε, CD3η, or CD3θ and CD3ζ.

In some embodiments, a CAR polypeptide described herein comprises the intracellular signaling domain of CD3ζ (including variants of CD3ζ, e.g., ITAM-mutated CD3ζ), CD3η, or CD3θ.

For example, a CAR polypeptide described herein comprises the intracellular signaling domain of CD3ζ. In one embodiment, the CD3 intracellular signaling sequence corresponds to a nucleotide sequence selected from SEQ ID NO: 6, 12, or 55; or comprises a sequence selected from SEQ ID NO: 6, 12, or 55; or comprises a sequence with at least 80%, at least 85%, at least 90%, at least 91%, at least 92%, at least 93%, at least 94%, at least 95%, at least 96%, at least 97%, at least 98%, at least 99%, or at least 100% sequence identity to a sequence selected from SEQ ID NO: 6, 12, or 55. In one embodiment, the CD3 intracellular signaling sequence corresponds to an amino acid sequence selected from SEQ ID NO: 24, 30, or 44; or comprises a sequence selected from SEQ ID NO: 24, 30, or 44; or comprises a sequence with at least 80%, at least 85%, at least 90%, at least 91%, at least 92%, at least 93%, at least 94%, at least 95%, at least 96%, at least 97%, at least 98%, at least 99%, or at least 100% sequence identity to a sequence selected from SEQ ID NO: 24, 30, or 44.

In further embodiments, a CAR polypeptide described herein comprises the intracellular signaling domain of CD3θ. In one embodiment, the CD3θ intracellular signaling sequence corresponds to the nucleotide sequence of SEQ ID NO: 18; or comprises the sequence of SEQ ID NO: 18; or comprises a sequence at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 18. In one embodiment, the CD3θ intracellular signaling sequence corresponds to the amino acid sequence of SEQ ID NO: 36; or comprises the sequence of SEQ ID NO: 36; or comprises a sequence at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 36.

A more detailed description of CARs and CART cells can be found in Maus et al. Blood 2014 123:2624-35; Reardon et al. Neuro-Oncology 2014 16:1441-1458; Hoyos et al. Haematologica 2012 97:1622; Byrd et al. J Clin Oncol 2014 32:3039-47; Maher et al. Cancer Res 2009 69:4559-4562; and Tamada et al. Clin Cancer Res 2012 18:6436-6445; each of which is incorporated by reference herein in its entirety.

In one embodiment, the CAR polypeptide further comprises a CD8 leader sequence. As used herein, a “leader sequence”, also known as leader RNA, refers to a region of an mRNA that is directly upstream of the initiation codon. A leader sequence can be important for the regulation of translation of a transcript.

In one embodiment, the CD8 leader sequence corresponds to a nucleotide sequence selected from SEQ ID NO: 2, 8, 14, or 47; or comprises a sequence selected from SEQ ID NO: 2, 8, 14, or 47; or comprises a sequence with at least 80%, at least 85%, at least 90%, at least 91%, at least 92%, at least 93%, at least 94%, at least 95%, at least 96%, at least 97%, at least 98%, at least 99%, or at least 100% sequence identity to a sequence selected from SEQ ID NO: 2, 8, 14, or 47. In one embodiment, the CD8 leader sequence corresponds to an amino acid sequence selected from SEQ ID NO: 20, 26, or 32; or comprises a sequence selected from SEQ ID NO: 20, 26, or 32; or comprises a sequence with at least 80%, at least 85%, at least 90%, at least 91%, at least 92%, at least 93%, at least 94%, at least 95%, at least 96%, at least 97%, at least 98%, at least 99%, or at least 100% sequence identity to a sequence selected from SEQ ID NO: 20, 26, or 32.

In some embodiments, a CAR polypeptide as described herein includes a signal peptide. Signal peptides can be derived from any protein that has an extracellular domain or is secreted. A CAR polypeptide as described herein may include any signal peptides known in the art. In some embodiments, the CAR polypeptide includes a CD8 signal peptide, e.g., a CD8 signal peptide corresponding to the amino acid sequence of SEQ ID NO: 20, 26, or 32, or comprising the amino acid sequence of SEQ ID NO: 20, 26, or 32, or comprising an amino acid sequence having at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 20, 26, or 32. In further embodiments, a CD8 signal peptide is encoded by a nucleotide sequence corresponding to a nucleotide sequence selected from SEQ ID NO: 2, 8, 14, or 47; or comprising a sequence selected from SEQ ID NO: 2, 8, 14, or 47; or comprising a sequence having at least 80%, at least 85%, at least 90%, at least 91%, at least 92%, at least 93%, at least 94%, at least 95%, at least 96%, at least 97%, at least 98%, at least 99%, or at least 100% sequence identity to a sequence selected from SEQ ID NO: 2, 8, 14, or 47.

In one embodiment, the CAR further comprises a linker domain. As used herein “linker domain” refers to an oligo- or polypeptide region from about 2 to 100 amino acids in length, which links together any of the domains/regions of the CAR as described herein. In some embodiment, linkers can include or be composed of flexible residues such as glycine and serine so that the adjacent protein domains are free to move relative to one another. Linker sequences useful for the invention include any linker sequence known in the art or as described herein, e.g., the linker sequence of SEQ ID NO: 41, 58, 61, 62, or 63. Longer linkers may be used when it is desirable to ensure that two adjacent domains do not sterically interfere with one another. Linkers may be cleavable or non-cleavable. Examples of cleavable linkers include 2A linkers (for example T2A), 2A-like linkers or functional equivalents thereof and combinations thereof. In one embodiment, the linker region is T2A derived from Thosea asigna virus. Non-limiting examples of linkers include linkers derived from Thosea asigna virus, and a linker derived from the internal ribosomal entry site (IRES) sequence.

In one embodiment, a CAR as described herein further comprises a reporter molecule, e.g., to permit for non-invasive imaging (e.g., positron-emission tomography PET scan). In a bispecific CAR that includes a reporter molecule, the first extracellular binding domain and the second extracellular binding domain can include different or the same reporter molecule. In a bispecific CAR T cell, the first CAR and the second CAR can express different or the same reporter molecule. In another embodiment, a CAR as described herein further comprises a reporter molecule (for example hygromycin phosphotransferase (hph)) that can be imaged alone or in combination with a substrate or chemical (for example 9-[4-[¹⁸F]fluoro-3-(hydroxymethyl)butyl]guanine ([¹⁸F]FHBG)). In another embodiment, a CAR as described herein further comprises nanoparticles at can be readily imaged using non-invasive techniques (e.g., gold nanoparticles (GNP) functionalized with ⁶⁴Cu²⁺). Labeling of CAR T cells for non-invasive imaging is reviewed, for example in Bhatnagar P, et al. Integr Biol. (Camb). 2013 January; 5(1): 231-238, and Keu K V, et al. Sdci Transl Med. 2017 Jan. 18; 9(373), which are incorporated herein by reference in their entireties.

GFP and mCherry are demonstrated herein as fluorescent tags useful for imaging a CAR expressed on a T cell (e.g., a CAR T cell). It is expected that essentially any fluorescent protein known in the art can be used as a fluorescent tag for this purpose. For clinical applications, the CAR need not include a fluorescent tag or fluorescent protein.

Another aspect of the invention relates to a CAR polypeptide comprising a sequence with at least 80%, at least 85%, at least 90%, at least 91%, at least 92%, at least 93%, at least 94%, at least 95%, at least 96%, at least 97%, at least 98%, at least 99%, or at least 100% sequence identity with a sequence selected from SEQ ID NO: 19, 25, 31, 39, 57, 64, or 65, or that is encoded by a nucleic acid comprising a nucleotide sequence with at least 80%, at least 85%, at least 90%, at least 91%, at least 92%, at least 93%, at least 94%, at least 95%, at least 96%, at least 97%, at least 98%, at least 99%, or at least 100% sequence identity with the sequence of SEQ ID NO: 1, 7, 13, or 45.

Another aspect of the invention relates to a CAR polypeptide comprising a sequence selected from SEQ ID NO: 19, 25, 31, 39, 57, 64, or 65, or that is encoded by a nucleic acid comprising a nucleotide sequence selected from SEQ ID NO: 1, 7, 13, or 45.

Another aspect of the invention relates to a CAR polypeptide comprising a sequence corresponding to a sequence selected from SEQ ID NO: 19, 25, 31, or 39, 57, 64, or 65, or that is encoded by a nucleic acid comprising a nucleotide sequence selected from SEQ ID NO: 1, 7, 13, or 45.

In some embodiments, a CAR polypeptide described herein comprises fully human sequences. Such polypeptides are produced according to methods known in the art.

Another aspect of the invention described herein relates to a polypeptide complex comprising two or more (e.g., two or more, three or more, four or more, five or more, six or more, seven or more, eight or more, nine or more, or ten or more) of any of the CAR polypeptides described herein. In one embodiment, the polypeptide complex comprises three of any of the CAR polypeptides described herein.

Nucleic Acids and Cells

In some embodiments, any of the CAR polypeptides described herein (e.g., a CAR polypeptide of SEQ ID NO: 19, 25, 31, 39, 57, 64, or 65) are encoded by a polynucleotide comprised in a viral vector. Optionally, a polynucleotide encoding a CAR polypeptide as described herein can be codon-optimized to enhance expression or stability. Codon optimization may be performed according to any standard methods known in the art.

Retroviruses, such as lentiviruses, provide a convenient platform for delivery of nucleic acid sequences encoding a gene, or chimeric gene of interest. A selected nucleic acid sequence can be inserted into a vector and packaged in retroviral particles using techniques known in the art. The recombinant virus can then be isolated and delivered to cells, e.g. in vitro or ex vivo. Retroviral systems are well known in the art and are described in, for example, U.S. Pat. No. 5,219,740; Kurth and Bannert (2010) “Retroviruses: Molecular Biology, Genomics and Pathogenesis” Calster Academic Press (ISBN:978-1-90455-55-4); and Hu and Pathak Pharmacological Reviews 2000 52:493-512; which are incorporated by reference herein in their entirety. Lentiviral system for efficient DNA delivery can be purchased from OriGene; Rockville, Md. In alternative embodiments, the CAR polypeptide of any of the CARs described herein are expressed in the mammalian cell via transfection or electroporation of an expression vector comprising nucleic acid encoding the CAR. Transfection or electroporation methods are known in the art.

Efficient expression of the CAR polypeptide of any of the CAR polypeptides described herein can be assessed using standard assays that detect the mRNA, DNA, or gene product of the nucleic acid encoding the CAR, such as RT-PCR, FACS, northern blotting, western blotting, ELISA, or immunohistochemistry.

In one embodiment, the CAR polypeptide of any of the CAR polypeptides described herein is constitutively expressed. In one embodiment, the CAR polypeptide of any of the CAR polypeptides described herein is encoded by recombinant nucleic acid sequence.

Another aspect of the invention relates to a mammalian cell comprising any of the CAR polypeptides described herein; or a nucleic acid encoding any of the CAR polypeptides described herein. In one embodiment, the mammalian cell comprises an antibody, antibody reagent, antigen-binding portion thereof, or any of the CAR polypeptides described herein, or a nucleic acid encoding such an antibody, antibody reagent, antigen-binding portion thereof, or any of the CAR polypeptides described herein. The mammalian cell or tissue can be of human, primate, hamster, rabbit, rodent, cow, pig, sheep, horse, goat, dog or cat origin, but any other mammalian cell may be used. In a preferred embodiment of any aspect, the mammalian cell is human. In one embodiment, the cell is a T cell or a natural killer (NK) cell. In alternate embodiments of any aspect, the cell is an immune cell. As used herein, “immune cell” refers to a cell that plays a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes. In some embodiments, the cell is a T cell; a NK cell; a NKT cell; lymphocytes, such as B cells and T cells; and myeloid cells, such as monocytes, macrophages, eosinophils, mast cells, basophils, and granulocytes.

The immune cell can be obtained from a subject having or diagnosed as having cancer, a plasma cell disorder, or an autoimmune disease or disorder. For example, the immune cell can be obtained from a subject having a cancer, e.g., multiple myeloma, smoldering myeloma, or Waldenstrom's macroglobulenemia. In some embodiments, the immune cell is obtained from a subject resistant to anti-BCMA therapy. Immune cells can also be obtained from allogeneic donors, which are non-genetically identical individuals of the same species as the intended recipients of the cells.

Immune cells (e.g., human immune cells) that can be used in the invention include autologous cells, obtained from the subject to whom the cells are later to be administered, after ex vivo modification and expansion. For example, the immune cells can be obtained from an individual having or diagnosed as having cancer, a plasma cell disorder, or autoimmune disease or disorder. Immune cells can also be obtained from allogeneic donors, which are non-genetically identical individuals of the same species as the intended recipients of the cells. Immune cells useful for the invention include T cells and NK cells.

Methods for obtaining T cells and NK are known in the art and can be useful for the engineered immune cells described herein. T cells and NK cells are typically obtained from peripheral blood that is collected from a subject by, e.g., venipuncture or withdrawal through an implanted port or catheter. Optionally, the blood can be obtained by a process including leukapheresis, in which white cells are obtained from the blood of a subject, while other blood components are returned to the subject. Blood or leukapheresis product (fresh or cryopreserved) is processed to enrich for T cells or NK cells using methods known in the art. For example, density gradient centrifugation (using, e.g., Ficoll) and/or counter-flow centrifugal elutriation can be carried out to enrich for mononuclear cells (including T cells or NK cells). In one example, for T cells, a T cell stimulation step employing, e.g., CD3/CD28 antibodies coated on magnetic beads or artificial antigen presenting cells (aAPCs) expressing, e.g., cell surface-bound anti-CD3 and anti-CD28 antibody fragments (see below), can further be carried out in order to stimulate T cells and to deplete other cells, e.g., B cells. The T cells of enriched T cell preparations can then be subject to genetic modification.

As an alternative to peripheral blood, tissues including bone marrow, lymph nodes, spleen, and tumors can be used as a source for T cells and NK cells. The T cells and NK cells can be of human, primate, hamster, rabbit, rodent, cow, pig, sheep, horse, goat, dog, or cat origin, but any other mammalian cell may be used. In a certain embodiments of any aspect, the T or NK cell is human.

An immune cell, e.g., a T cell or NK cell, can be engineered to comprise any of the CAR polypeptides described herein (e.g., the CAR polypeptide of SEQ ID NO: 19, 25, 31, 39, 57, 64, or 65); or a nucleic acid encoding any of the CAR polypeptides described herein (e.g., a nucleic acid encoding the CAR polypeptide of SEQ ID NO: 19, 25, 31, 39, 57, 64, or 65).

The invention furthermore provides compositions and methods for treating and preventing diseases and conditions including, e.g., cancer, autoimmune diseases or disorders, or plasma cell diseases or disorders. These methods include the use of an immune cell (e.g., a T cell or an NK cell) including a CAR polypeptide, or a nucleic acid encoding said CAR, as described herein, and administering the modified immune cell to a subject to treat, e.g., cancer. In some embodiments of any of the aspect, the modified immune cell (e.g., a T cell or an NK cell including one or more additional modification as described herein) is stimulated and/or activated prior to administration to the subject.

Therapeutic Methods

The invention provides compositions and methods for treating a cancer (e.g., multiple myeloma), a plasma cell disorder, or an autoimmune disease or disorder using the CAR polypeptides described herein. In particular embodiments, the cancer, e.g., multiple myeloma, smoldering myeloma, or Waldenstrom's macroglobulenemia, expresses BCMA and/or TACI. In further embodiments, the cancer, e.g., multiple myeloma, smoldering myeloma, or Waldenstrom's macroglobulenemia, has reduced or eliminated BCMA expression. Furthermore, the invention provides methods for treating a cancer, e.g., multiple myeloma, smoldering myeloma, or Waldenstrom's macroglobulenemia, in a patient resistant to anti-BCMA therapy.

“Cancer” as used herein can refer to a hyperproliferation of cells whose unique trait—loss of normal cellular control—results in unregulated growth, lack of differentiation, local tissue invasion, and metastasis, and can be leukemia, lymphoma, multiple myeloma, or a solid tumor. Non-limiting examples of leukemia include acute myeloid leukemia (AML), Chronic myeloid leukemia (CML), Acute lymphocytic leukemia (ALL), and Chronic lymphocytic leukemia (CLL). In one embodiment, the cancer is ALL or CLL. Non-limiting examples of lymphoma include Diffuse large B-cell lymphoma (DLBCL), Follicular lymphoma, Chronic lymphocytic leukemia (CLL), Small lymphocytic lymphoma (SLL), Mantle cell lymphoma (MCL), Marginal zone lymphomas, Burkitt lymphoma, hairy cell leukemia (HCL), and Waldenstrom's macroglobulinemia. In one embodiment, the cancer is DLBCL or Follicular lymphoma. Non-limiting examples of solid tumors include Adrenocortical Tumor, Alveolar Soft Part Sarcoma, Carcinoma, Chondrosarcoma, Colorectal Carcinoma, Desmoid Tumors, Desmoplastic Small Round Cell Tumor, Endocrine Tumors, Endodermal Sinus Tumor, Epithelioid Hemangioendothelioma, Ewing Sarcoma, Germ Cell Tumors (Solid Tumor), Giant Cell Tumor of Bone and Soft Tissue, Hepatoblastoma, Hepatocellular Carcinoma, Melanoma, Nephroma, Neuroblastoma, Non-Rhabdomyosarcoma Soft Tissue Sarcoma (NRSTS), Osteosarcoma, Paraspinal Sarcoma, Renal Cell Carcinoma, Retinoblastoma, Rhabdomyosarcoma, Synovial Sarcoma, and Wilms Tumor. Solid tumors can be found in bones, muscles, or organs, and can be sarcomas or carcinomas. It is contemplated that any aspect of the invention described herein can be used to treat all types of cancers, including cancers not listed in the instant application. As used herein, the term “tumor” refers to an abnormal growth of cells or tissues, e.g., of malignant type or benign type.

As used herein, an “autoimmune disease or disorder” is characterized by the inability of one's immune system to distinguish between a foreign cell and a healthy cell. This results in one's immune system targeting one's healthy cells for programmed cell death. Non-limiting examples of an autoimmune disease or disorder include inflammatory arthritis, type 1 diabetes mellitus, multiples sclerosis, psoriasis, inflammatory bowel diseases, SLE, and vasculitis, allergic inflammation, such as allergic asthma, atopic dermatitis, and contact hypersensitivity, rheumatoid arthritis, multiple sclerosis (MS), systemic lupus erythematosus, Graves' disease (overactive thyroid), Hashimoto's thyroiditis (underactive thyroid), chronic graft v. host disease, hemophilia with antibodies to coagulation factors, celiac disease, Crohn's disease and ulcerative colitis, Guillain-Barre syndrome, primary biliary sclerosis/cirrhosis, sclerosing cholangitis, autoimmune hepatitis, Raynaud's phenomenon, scleroderma, Sjogren's syndrome, Goodpasture's syndrome, Wegener's granulomatosis, polymyalgia rheumatica, temporal arteritis/giant cell arteritis, chronic fatigue syndrome CFS), psoriasis, autoimmune Addison's Disease, ankylosing spondylitis, Acute disseminated encephalomyelitis, antiphospholipid antibody syndrome, aplastic anemia, idiopathic thrombocytopenic purpura, Myasthenia gravis, opsoclonus myoclonus syndrome, optic neuritis, Ord's thyroiditis, pemphigus, pernicious anaemia, polyarthritis in dogs, Reiter's syndrome, Takayasu's arteritis, warm autoimmune hemolytic anemia, Wegener's granulomatosis and fibromyalgia (FM).

In one embodiment, the mammalian cell is obtained for a patient having an immune system disorder that results in abnormally low activity of the immune system, or immune deficiency disorders, which hinders one's ability to fight a foreign cell, (i.e., a virus or bacterial cell).

A plasma cell is a white blood cell produces from B lymphocytes which function to generate and release antibodies needed to fight infections. As used herein, a “plasma cell disorder or disease” is characterized by abnormal multiplication of a plasma cell. Abnormal plasma cells are capable of “crowding out” healthy plasma cells, which results in a decreased capacity to fight a foreign object, such as a virus or bacterial cell. Non-limiting examples of plasma cell disorders include amyloidosis, Waldenstrom's macroglobulinemia, osteosclerotic myeloma (POEMS syndrome), Monoclonal gammopathy of unknown significance (MGUS), smoldering myeloma, solitary plasmacytoma, and plasma cell myeloma.

One aspect of the invention described herein relates to a method to a method of treating cancer, a plasma cell disorder, amyloidosis, or an autoimmune disease or disorder in a subject, the method comprising: engineering a T cell to comprise any of the CAR polypeptides described herein on the T cell surface; administering the engineered T cell to the subject.

Another aspect of the invention described herein relates to a method of treating cancer, a plasma cell disorder, or an autoimmune disease or disorder in a subject, the method comprising administering a cell comprising any of the CAR polypeptides described herein, or a nucleic acid encoding any of the CAR polypeptides described herein.

In one embodiment, the method further comprises activating or stimulating the CAR T prior to administering the cell to the subject, e.g., according to a method as described elsewhere herein.

The invention described herein provides methods of treating a cancer, a plasma cell disorder, or an autoimmune disease or disorder in a subject comprising administering to the subject an immune cell (e.g., a T or NK cell), or a composition thereof, comprising a CAR polypeptide as described herein (e.g., the CAR polypeptide of SEQ ID NO: 19, 25, 31, 39, 57, 64, or 65) and/or a nucleic acid capable of encoding said CAR. In some embodiments, the cancer comprises cells expressing B cell activating factor (BAFF), B cell maturation antigen (BCMA), and/or transmembrane activator and CAML interactor (TACI). In one embodiment, the cancer cell comprises the tumor antigens BAFF+, BCMA+, and/or TACI+ cancer. In some embodiments, the subject is resistant to anti-BCMA therapy. In some embodiments, the cancer comprises cells with reduced BCMA expression. In further embodiments, the plasma cell disorder is amyloidosis. In some embodiments, the autoimmune disorder is hemophilia with antibodies to coagulation factors, myasthenia gravis, multiple sclerosis, or chronic graft versus host disease.

In one embodiment, cancer is a myeloma, e.g., multiple myeloma, smoldering myeloma, or Waldenstrom's macroglobulenemia.

Furthermore, the CAR polypeptides described herein can be used for methods of treating or preventing transplant rejection in a subject. In some embodiments, the subject has high levels of anti-HLA antibodies.

Administration

In some embodiments, the methods described herein relate to treating a subject having or diagnosed as having cancer, a plasma cell disease or disorder, or an autoimmune disease or disorder with a mammalian cell comprising any of the CAR polypeptides described herein, or a nucleic acid encoding any of the CAR polypeptides described herein. As used herein, a “CAR T cell as described herein” refers to a mammalian cell comprising any of the CAR polypeptides described herein, or a nucleic acid encoding any of the CAR polypeptides described herein. As used herein, a “condition” refers to a cancer, a plasma cell disease or disorder, or an autoimmune disease or disorder. Subjects having a condition can be identified by a physician using current methods of diagnosing the condition. Symptoms and/or complications of the condition, which characterize these conditions and aid in diagnosis are well known in the art and include but are not limited to, fatigue, persistent infections, and persistent bleeding. Tests that may aid in a diagnosis of, e.g. the condition, but are not limited to, blood screening and bone marrow testing, and are known in the art for a given condition. A family history for a condition, or exposure to risk factors for a condition can also aid in determining if a subject is likely to have the condition or in making a diagnosis of the condition.

The compositions described herein can be administered to a subject having or diagnosed as having a condition. In some embodiments, the methods described herein comprise administering an effective amount of activated CAR T cells described herein to a subject in order to alleviate a symptom of the condition. As used herein, “alleviating a symptom of the condition” is ameliorating any condition or symptom associated with the condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. In one embodiment, the compositions described herein are administered systemically or locally. In a preferred embodiment, the compositions described herein are administered intravenously. In another embodiment, the compositions described herein are administered at the site of the tumor.

The term “effective amount” as used herein refers to the amount of activated CAR T cells needed to alleviate at least one or more symptom of the disease or disorder, and relates to a sufficient amount of the cell preparation or composition to provide the desired effect. The term “therapeutically effective amount” therefore refers to an amount of activated CAR T cells that is sufficient to provide a particular anti-condition effect when administered to a typical subject. An effective amount as used herein, in various contexts, would also include an amount sufficient to delay the development of a symptom of the disease, alter the course of a symptom disease (for example but not limited to, slowing the progression of a condition), or reverse a symptom of the condition. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of activated CAR T cells, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for bone marrow testing, among others. The dosage can be determined by a physician and adjusted, as necessary, to suit observed effects of the treatment.

In one aspect of the invention, the technology described herein relates to a pharmaceutical composition comprising activated CAR T cells as described herein, and optionally a pharmaceutically acceptable carrier. The active ingredients of the pharmaceutical composition at a minimum comprise activated CAR T cells as described herein. In some embodiments, the active ingredients of the pharmaceutical composition consist essentially of activated CAR T cells as described herein. In some embodiments, the active ingredients of the pharmaceutical composition consist of activated CAR T cells as described herein. Pharmaceutically acceptable carriers for cell-based therapeutic formulation include saline and aqueous buffer solutions, Ringer's solution, and serum component, such as serum albumin, HDL and LDL. The terms such as “excipient”, “carrier”, “pharmaceutically acceptable carrier” or the like are used interchangeably herein.

In some embodiments, the pharmaceutical composition comprising activated CAR T cells as described herein can be a parenteral dose form. Since administration of parenteral dosage forms typically bypasses the patient's natural defenses against contaminants, the components apart from the CAR T cells themselves are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. Any of these can be added to the activated CAR T cells preparation prior to administration.

Suitable vehicles that can be used to provide parenteral dosage forms of activated CAR T cells as disclosed within are well known to those skilled in the art. Examples include, without limitation: saline solution; glucose solution; aqueous vehicles including but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Dosage

“Unit dosage form” as the term is used herein refers to a dosage for suitable one administration. By way of example a unit dosage form can be an amount of therapeutic disposed in a delivery device, e.g., a syringe or intravenous drip bag. In one embodiment, a unit dosage form is administered in a single administration. In another, embodiment more than one unit dosage form can be administered simultaneously.

In some embodiments, the activated CAR T cells described herein are administered as a monotherapy, i.e., another treatment for the condition is not concurrently administered to the subject.

A pharmaceutical composition comprising the T cells described herein can generally be administered at a dosage of 10⁴ to 10⁹ cells/kg body weight, in some instances 10⁵ to 10⁶ cells/kg body weight, including all integer values within those ranges. If necessary, T cell compositions can also be administered multiple times at these dosages. The cells can be administered by using infusion techniques that are commonly known in immunotherapy (see, e.g., Rosenberg et al., New Eng. J. of Med. 319:1676, 1988).

In certain aspects, it may be desired to administer activated CAR T cells to a subject and then subsequently redraw blood (or have an apheresis performed), activate T cells therefrom as described herein, and reinfuse the patient with these activated and expanded T cells. This process can be carried out multiple times every few weeks. In certain aspects, T cells can be activated from blood draws of from 10cc to 400cc. In certain aspects, T cells are activated from blood draws of 20cc, 30cc, 40cc, 50cc, 60cc, 70cc, 80cc, 90cc, or 100cc.

Modes of administration can include, for example intravenous (i.v.) injection or infusion. The compositions described herein can be administered to a patient transarterially, intratumorally, intranodally, or intramedullary. In some embodiments, the compositions of T cells may be injected directly into a tumor, lymph node, or site of infection. In one embodiment, the compositions described herein are administered into a body cavity or body fluid (e.g., ascites, pleural fluid, peritoneal fluid, or cerebrospinal fluid).

In a particular exemplary aspect, subjects may undergo leukapheresis, wherein leukocytes are collected, enriched, or depleted ex vivo to select and/or isolate the cells of interest, e.g., T cells. These T cell isolates can be expanded by contact with an aAPC as described herein, e.g., an aAPC expressing anti-CD28 and anti-CD3 CDRs as described herein and treated such that one or more CAR constructs of the invention may be introduced, thereby creating a CAR T cell. Subjects in need thereof can subsequently undergo standard treatment with high dose chemotherapy followed by peripheral blood stem cell transplantation. Following or concurrent with the transplant, subjects can receive an infusion of the expanded CAR T cells. In one embodiment, expanded cells are administered before or following surgery.

In some embodiments, lymphodepletion is performed on a subject prior to administering one or more CAR T cell as described herein. In such embodiments, the lymphodepletion can comprise administering one or more of melphalan, Cytoxan, cyclophosphamide, and fludarabine.

The dosage of the above treatments to be administered to a patient will vary with the precise nature of the condition being treated and the recipient of the treatment. The scaling of dosages for human administration can be performed according to art-accepted practices.

In some embodiments, a single treatment regimen is required. In others, administration of one or more subsequent doses or treatment regimens can be performed. For example, after treatment biweekly for three months, treatment can be repeated once per month, for six months or a year or longer. In some embodiments, no additional treatments are administered following the initial treatment.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

Combination Therapy

The activated CAR T cells described herein can be used in combination with other known agents and therapies. In one embodiment, the subject is further administered an anti-BCMA therapy. In one embodiment, the subject is resistant to anti-BCMA therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The activated CAR T cells described herein and the at least one additional therapeutic agent can be administered simultaneously, in the same or in separate compositions, or sequentially. For sequential administration, the CAR-expressing cell described herein can be administered first, and the additional agent can be administered second, or the order of administration can be reversed. The CAR T therapy and/or other therapeutic agents, procedures or modalities can be administered during periods of active disorder, or during a period of remission or less active disease. The CAR T therapy can be administered before another treatment, concurrently with the treatment, post-treatment, or during remission of the disorder.

When administered in combination, the activated CAR T cells and the additional agent (e.g., second or third agent), or all, can be administered in an amount or dose that is higher, lower or the same as the amount or dosage of each agent used individually, e.g., as a monotherapy. In certain embodiments, the administered amount or dosage of the activated CAR T cells, the additional agent (e.g., second or third agent), or all, is lower (e.g., at least 20%, at least 30%, at least 40%, or at least 50%) than the amount or dosage of each agent used individually. In other embodiments, the amount or dosage of the activated CAR T cells, the additional agent (e.g., second or third agent), or all, that results in a desired effect (e.g., treatment of cancer) is lower (e.g., at least 20%, at least 30%, at least 40%, or at least 50% lower) than the amount or dosage of each agent individually required to achieve the same therapeutic effect. In further embodiments, the activated CAR T cells described herein can be used in a treatment regimen in combination with surgery, chemotherapy, radiation, an mTOR pathway inhibitor, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, rapamycin, mycophenolic acid, steroids, FR901228, cytokines, or a peptide vaccine, such as that described in Izumoto et al. 2008 J Neurosurg 108:963-971.

In one embodiment, the activated CAR T cells described herein can be used in combination with a checkpoint inhibitor. Exemplary checkpoint inhibitors include anti-PD-1 inhibitors (Nivolumab, MK-3475, Pembrolizumab, Pidilizumab, AMP-224, AMP-514), anti-CTLA4 inhibitors (Ipilimumab and Tremelimumab), anti-PDL1 inhibitors (Atezolizumab, Avelomab, MSB0010718C, MED14736, and MPDL3280A), and anti-TIM3 inhibitors.

In one embodiment, the activated CAR T cells described herein can be used in combination with a chemotherapeutic agent. Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide). General chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®). Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil Nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HC1 (Treanda®). Exemplary mTOR inhibitors include, e.g., temsirolimus; ridaforolimus (formally known as deferolimus, (IR,2R,45)-4-[(2R)-2[(1R,95,125,15R,16E,18R,19R,21R,235,24E,26E,28Z,305,325,35R)-1,18-dihydroxy-19,30-dimethoxy-15,17,21,23,29,35-hexamethyl-2,3,10,14,20-pentaoxo-11,36-dioxa-4-azatricyclo[30.3.1.04′9] hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RADOOI); rapamycin (AY22989, Sirolimus®); simapimod (CAS 164301-51-3); emsirolimus, (5-{2,4-Bis[(35)-3-methylmorpholin-4-yl]pyrido[2,3-(i]pyrimidin-7-yl}-2-methoxyphenyl)methanol (AZD8055); 2-Amino-8-[iraw5,-4-(2-hydroxyethoxy)cyclohexyl]-6-(6-methoxy-3-pyridinyl)-4-methyl-pyrido[2,3-JJpyrimidin-7(8H)-one (PF04691502, CAS 1013101-36-4); and N2-[1,4-dioxo-4-[[4-(4-oxo-8-phenyl-4H-1-benzopyran-2-yl)morpholinium-4-yl]methoxy]butyl]L-arginylglycyl-L-a-aspartyl-L-serine-, inner salt (SF1126, CAS 936487-67-1), and XL765. Exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon γ, CAS 951209-71-5, available from IRX Therapeutics). Exemplary anthracyclines include, e.g., doxorubicin (Adriamycin® and Rubex®); bleomycin (Ienoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin. Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®). Exemplary proteosome inhibitors include bortezomib (Velcade®); carfilzomib (PX-171-007, (5)-4-Methyl-N-((5)-I-(((5)-4-methyl-I—((R)-2-methyloxiran-2-yl)-I-oxopentan-2-yl)amino)-I-oxo-3-phenylpropan-2-yl)-2-((5)-2-(2-morpholinoacetamido)-4-phenylbutanamido)-pentanamide); marizomib (NPT0052); ixazomib citrate (MLN-9708); delanzomib (CEP-18770); and O-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912).

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003).

In an embodiment, activated CAR T cells described herein are administered to a subject in combination with a molecule that decreases the activity and/or level of a molecule targeting GITR and/or modulating GITR functions, a molecule that decreases the Treg cell population, an mTOR inhibitor, a GITR agonist, a kinase inhibitor, a non-receptor tyrosine kinase inhibitor, a CDK4 inhibitor, and/or a BTK inhibitor.

Efficacy

The efficacy of activated CAR T cells in, e.g. the treatment of a condition described herein, or to induce a response as described herein (e.g. a reduction in cancer cells) can be determined by the skilled clinician. However, a treatment is considered “effective treatment,” as the term is used herein, if one or more of the signs or symptoms of a condition described herein is altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Treatment according to the methods described herein can reduce levels of a marker or symptom of a condition, e.g. by at least 10%, at least 15%, at least 20%, at least 25%, at least 30%, at least 40%, at least 50%, at least 60%, at least 70%, at least 80% or at least 90% or more.

Efficacy can also be measured by a failure of an individual to worsen as assessed by hospitalization, or need for medical interventions (i.e., progression of the disease is halted). Methods of measuring these indicators are known to those of skill in the art and/or are described herein.

Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy of a given approach can be assessed in animal models of a condition described herein, for example treatment of ALL. When using an experimental animal model, efficacy of treatment is evidenced when a statistically significant change in a marker is observed.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A chimeric antigen receptor (CAR) polypeptide comprising:
   a) two or more extracellular domains, each comprising a Tumor Necrosis Factor (TNF) superfamily receptor ligand or a portion thereof;
   b) a transmembrane domain; and
   c) an intracellular signaling domain.
2. The CAR polypeptide of paragraph 1, wherein the transmembrane domain comprises a hinge/transmembrane domain.
3. The CAR polypeptide of paragraph 1 or 2, further comprising one or more co-stimulatory domains.
4. The CAR polypeptide of any one of paragraphs 1-3, wherein the TNF superfamily receptor ligand is A Proliferation-Inducing Ligand (APRIL).
5. The CAR polypeptide of any one of paragraphs 1-3, wherein the TNF superfamily receptor ligand is TNF-alpha, lymphotoxin beta, OX40 ligand (OX40L), CD154, Fas ligand (FasL), LIGHT, TNF-like ligand 1A (TL1A), CD70, Siva, CD153, 4-1BB ligand (4-1BBL), TNF-related apoptosis-inducing ligand (TRAIL), receptor activator of nuclear factor kappa-B ligand (RANKL), TNF-related weak inducer of apoptosis (TWEAK), B cell activating factor (BAFF), calcium modulating ligand (CAMLG), nerve growth factor (NGF), brain-derived neurotrophic factor (BDNF), neurotrophin-3 (NT-3), neurotrophin-4 (NT-4), glucocorticoid-induced TNF receptor (TNFR)-related protein (GITR) ligand, or ectodysplasin A2 (EDA-A2).
6. The CAR polypeptide of any one of paragraphs 1-5, wherein the two or more extracellular domains each comprise a portion of the same TNF superfamily receptor ligand.
7. The CAR polypeptide of any one of paragraphs 1-5, wherein at least two of the extracellular domains each comprise a portion of different TNF superfamily receptor ligands.
8. The CAR polypeptide of any one of paragraphs 1-7, wherein the two or more extracellular domains are connected to each other by one or more linker sequences.
9. The CAR polypeptide of paragraph 8, wherein each linker sequence comprises an amino acid sequence having at least 90% sequence identity to the amino acid sequence of SEQ ID NO: 41, 58, 61, 62, or 63.
10. The CAR polypeptide of paragraph 8 or 9, wherein each linker sequence comprises the amino acid sequence of SEQ ID NO: 41, 58, 61, 62, or 63.
11. The CAR polypeptide of any one of paragraphs 1-10, further comprising a leader sequence.
12. The CAR polypeptide of paragraph 11, wherein the leader sequence is a CD8 leader sequence.
13. The CAR polypeptide of paragraph 12, wherein the CD8 leader sequence comprises the sequence of SEQ ID NO: 20, 26, or 32.
14. The CAR polypeptide of any one of paragraphs 4 and 6-13, wherein the portion of APRIL does not comprise a lysine-rich region of APRIL.
15. The CAR polypeptide of any one of paragraphs 4 and 6-14, wherein the portion of APRIL does not comprise a furin cleavage site.
16. The CAR polypeptide of any one of paragraphs 4 and 6-15, wherein the portion of APRIL comprises the sequence of SEQ ID NO: 21, 27, 33, or 40.
17. The CAR polypeptide of any one of paragraphs 1-16, wherein the hinge and transmembrane domain comprises the hinge and transmembrane domain of CD28, CD8, or 4-1 BB.
18. The CAR polypeptide of paragraph 17, wherein the CD8 hinge and transmembrane domain comprises the amino acid sequence of SEQ ID NO: 22 or 42.
19. The CAR polypeptide of paragraph 17, wherein the 4-1 BB hinge and transmembrane domain comprises the amino acid sequence of SEQ ID NO: 28 or 34.
20. The CAR polypeptide of any one of paragraphs 1-19, wherein the intracellular signaling domain comprises the intracellular signaling domain of CD3ζ, CD3ε, or CD3θ.
21. The CAR polypeptide of paragraph 20, wherein the CD3 intracellular signaling domain comprises the amino acid sequence of SEQ ID NO: 24, 30, or 44.
22. The CAR polypeptide of paragraph 20, wherein the CD3θ intracellular signaling domain comprises the amino acid sequence of SEQ ID NO: 36.
23. The CAR polypeptide of any one of paragraphs 1-22, wherein the co-stimulatory domain comprises the intracellular domain of 4-1 BB, CD28, CD27, ICOS, or OX40.
24. The CAR polypeptide of any one of paragraphs 1-23, wherein the co-stimulatory domain is the intracellular domain of 4-1 BB.
25. The CAR polypeptide of paragraph 24, wherein the intracellular domain of 4-1 BB comprises the amino acid sequence of SEQ ID NO: 23, 29, 35, or 43.
26. The CAR polypeptide of any one of paragraphs 1-25, wherein the CAR polypeptide comprises three extracellular domains, each comprising a portion of a TNF superfamily receptor ligand.
27. A CAR polypeptide comprising at least 95% sequence identity with the sequence of SEQ ID NO: 39, 57, 64, or 65, or that is encoded by a sequence comprising at least 95% sequence identity with the sequence of SEQ ID NO: 45.
28. A CAR polypeptide comprising the sequence of SEQ ID NO: 39, 57, 64, or 65, or that is encoded by the sequence of SEQ ID NO: 45.
29. A CAR polypeptide comprising a sequence corresponding to the sequence of SEQ ID NO: 39, 57, 64, or 65, or that is encoded by a sequence of SEQ ID NO: 45.
30. A polypeptide complex comprising two or more of the CAR polypeptides of any one of paragraphs 1-29.
31. The polypeptide complex of paragraph 30, wherein the polypeptide complex comprises three CAR polypeptides of any one of paragraphs 1-29.
32. A mammalian cell comprising:
   a) the CAR polypeptide of any one of paragraphs 1-29;
   b) a nucleic acid encoding the CAR polypeptide of any one of paragraphs 1-29; or
   c) the polypeptide complex of paragraph 30 or 31.
33. The cell of paragraph 32, wherein the cell is a T or natural killer (NK) cell.
34. The cell of paragraph 32 or 33, wherein the cell is a human cell.
35. The cell of any one of paragraphs 32-34, wherein the cell is obtained from an individual having or diagnosed as having cancer, a plasma cell disorder, or an autoimmune disease or disorder.
36. A method of treating a cancer, a plasma cell disorder, amyloidosis, an autoimmune disease or disorder, or transplant rejection in a subject, the method comprising:
   a) engineering a T cell to comprise the CAR of any one of paragraphs 1-29 on the T cell surface; and
   b) administering the engineered T cell to the subject.
37. A method of treating a cancer, a plasma cell disorder, an autoimmune disease or disorder, or transplant rejection in a subject, the method comprising administering the cell of any one of paragraphs 32-35 to the subject.
38. The method of paragraph 36 or 37, wherein the cancer is BAFF+, B cell maturation antigen (BCMA)+ and/or transmembrane activator and calcium modulating ligand (CAML) interactor (TACI)+.
39. The method of any one of paragraphs 36-38, wherein the subject is further administered an anti-BCMA therapy.
40. The method of any one of paragraphs 36-39, wherein the subject is resistant to anti-BCMA therapies.
41. The method of any one of paragraphs 36-40, wherein the cancer is multiple myeloma, smoldering myeloma, or Waldenstrom's macroglobulenemia.
42. The method of any one of paragraphs 36-41, wherein the autoimmune disease is selected from the group consisting of hemophilia with antibodies to coagulation factors, myasthenia gravis, multiple sclerosis, and chronic graft versus host disease.
43. The method of any one of paragraphs 36-42, wherein the subject has high levels of anti-human leukocyte antigen (HLA) antibodies.
44. A composition comprising the CAR polypeptide of any one of paragraphs 1-29, the polypeptide complex of paragraph 30 or 31, or the cell of any one of paragraphs 32-35 formulated for the treatment of cancer.
45. The composition of paragraph 44, further comprising a pharmaceutically acceptable carrier.
46. A method of treating a subject resistant to anti-BCMA therapy, the method comprising administering to the subject an immune cell comprising a CAR and/or a polynucleotide encoding the CAR, wherein the CAR comprises an extracellular target-binding domain comprising two or more APRIL domains.
47. The method of paragraph 46, wherein the subject has a cancer, a plasma cell disorder, an autoimmune disease or disorder, or transplant rejection.
48. The method of paragraph 47, wherein the cancer comprises cells expressing BCMA and/or TACI.
49. The method of paragraph 47 or 48, wherein the cancer comprises cells with reduced BCMA expression.
50. The method of any one of paragraphs 47-49, wherein the cancer is a myeloma or Waldenstrom's macroglobulinemia.
51. The method of any one of paragraphs 47-50, wherein the cancer is multiple myeloma or smoldering myeloma.
52. The method of paragraph 47, wherein the plasma cell disorder is amyloidosis.
53. The method of paragraph 47, wherein the autoimmune disease or disorder is selected from the group consisting of hemophilia with antibodies to coagulation factors, myasthenia gravis, multiple sclerosis, and chronic graft versus host disease.
54. The method of paragraph 47, wherein the subject has high levels of anti-HLA antibodies.
55. The method of any one of paragraphs 46-54, wherein the CAR comprises a transmembrane domain and an intracellular signaling domain.
56. The method of any one of paragraphs 46-55, wherein the CAR further comprises one or more co-stimulatory domains.
57. The method of paragraph 55 or 56, wherein the transmembrane domain comprises a hinge/transmembrane domain.
58. The method of paragraph 57, wherein the hinge/transmembrane domain comprises the hinge/transmembrane domain of an immunoglobulin-like protein, CD28, CD8, or 4-1 BB.
59. The method of paragraph 57 or 58, wherein the hinge/transmembrane domain comprises the hinge/transmembrane domain of CD8, optionally comprising the amino acid sequence of SEQ ID NO: 22 or 42.
60. The method of paragraph 57 or 58, wherein the hinge/transmembrane domain comprises the hinge/transmembrane domain of 4-1 BB, optionally comprising the amino acid sequence of SEQ ID NO: 28 or 34.
61. The method of any one of paragraphs 55-60, wherein the intracellular signaling domain comprises the intracellular signaling domain of TCFζ, FcRγ, FcRβ, CD3γ, CD3θ, CD3ζ, CD3ε, CD3η, CD3ζ, CD22, CD79a, CD79b, or CD66d.
62. The method of paragraph 61, wherein the intracellular signaling domain comprises the intracellular signaling domain of CD3ζ, optionally comprising the amino acid sequence of SEQ ID NO: 24, 30, or 44.
63. The method of paragraph 61, wherein the intracellular signaling domain comprises the intracellular signaling domain of CD3θ, optionally comprising the amino acid sequence of SEQ ID NO: 36.
64. The method of any one of paragraphs 56-63, wherein the co-stimulatory domain comprises the co-stimulatory domain of 4-1 BB, CD27, CD28, or OX40.
65. The method of paragraph 64, wherein the co-stimulatory domain comprises the co-stimulatory domain of 4-1 BB, optionally comprising the amino acid sequence of SEQ ID NO: 23, 29, 35, or 43.
66. The method of any one of paragraphs 46-65, wherein each APRIL domain comprises an amino acid sequence having at least 90% sequence identity to the amino acid sequence of SEQ ID NO: 21, 27, 33, or 40.
67. The method of paragraph 66, wherein each APRIL domain comprises the amino acid sequence of SEQ ID NO: 21, 27, 33, or 40.
68. The method of any one of paragraphs 46-67, wherein the APRIL domains are connected to each other by one or more linker sequences.
69. The method of paragraph 68, wherein the linker sequence comprises an amino acid sequence having at least 90% sequence identity to the amino acid sequence of SEQ ID NO: 41, 58, 61, 62, or 63.
70. The method of paragraph 68 or 69, wherein the linker sequence comprises the amino acid sequence of SEQ ID NO: 41, 58, 61, 62, or 63.
71. The method of any one of paragraphs 46-70, wherein the extracellular target-binding domain comprises three APRIL domains.
72. The method of any one of paragraphs 46-71, wherein the extracellular target-binding domain comprises an amino acid sequence having at least 90% sequence identity to the amino acid sequence of SEQ ID NO: 56 or 59.
73. The method of any one of paragraphs 46-72, wherein the extracellular target-binding domain comprises the amino acid sequence of SEQ ID NO: 56 or 59.
74. The method of any one of paragraphs 46-73, wherein the CAR comprises an amino acid sequence having at least 90% sequence identity to the amino acid sequence of SEQ ID NO: 39, 57, 64, or 65.
75. The method of any one of paragraphs 46-74, wherein the CAR comprises the amino acid sequence of SEQ ID NO: 39, 57, 64, or 65.
76. The method of any one of paragraphs 46-75, wherein the polynucleotide encoding the CAR further comprises a suicide gene. 77. The method of any one of paragraphs 46-76, wherein the polynucleotide encoding the CAR further comprises a sequence encoding a signal sequence.
78. The method of any one of paragraphs 46-77, wherein the immune cell is a T or NK cell.
79. The method of any one of paragraphs 46-78, wherein the immune cell is a human cell.
80. A CAR comprising an extracellular target-binding domain comprising three APRIL domains.
81. The CAR of paragraph 80, wherein the CAR comprises a transmembrane domain and an intracellular signaling domain.
82. The CAR of paragraph 81, wherein the CAR further comprises one or more co-stimulatory domains.
83. The CAR of paragraph 81 or 82, wherein the transmembrane domain comprises a hinge/transmembrane domain.
84. The CAR of paragraph 83, wherein the hinge/transmembrane domain comprises the hinge/transmembrane domain of an immunoglobulin-like protein, CD28, CD8, or 4-1 BB.
85. The CAR of paragraph 83 or 84, wherein the hinge/transmembrane domain comprises the hinge/transmembrane domain of CD8, optionally comprising the amino acid sequence of SEQ ID NO: 22 or 42.
86. The CAR of paragraph 83 or 84, wherein the hinge/transmembrane domain comprises the hinge/transmembrane domain of 4-1 BB, optionally comprising the amino acid sequence of SEQ ID NO: 28 or 34.
87. The CAR of any one of paragraphs 81-86, wherein the intracellular signaling domain comprises the intracellular signaling domain of TCFζ, FcRγ, FcRβ, CD3γ, CD3θ, CD3ζ, CD3ε, CD3η, CD3ζ, CD22, CD79a, CD79b, or CD66d.
88. The CAR of paragraph 87, wherein the intracellular signaling domain comprises the intracellular signaling domain of CD3ζ, optionally comprising the amino acid sequence of SEQ ID NO: 24, 30, or 44.
89. The CAR of paragraph 87, wherein the intracellular signaling domain comprises the intracellular signaling domain of CDT), optionally comprising the amino acid sequence of SEQ ID NO: 36.
90. The CAR of any one of paragraphs 82-89, wherein the co-stimulatory domain comprises the co-stimulatory domain of 4-1 BB, CD27, CD28, or OX40.
91. The CAR of paragraph 90, wherein the co-stimulatory domain comprises the co-stimulatory domain of 4-1 BB, optionally comprising the amino acid sequence of SEQ ID NO: 23, 29, 35, or 43.
92. The CAR of any one of paragraphs 80-91, wherein each APRIL domain comprises an amino acid sequence having at least 90% sequence identity to the amino acid sequence of SEQ ID NO: 21, 27, 33, or 40.
93. The CAR of paragraph 92, wherein each APRIL domain comprises the amino acid sequence of SEQ ID NO: 21, 27, 33, or 40.
94. The CAR of any one of paragraphs 80-93, wherein the APRIL domains are connected to each other by one or more linker sequences.
95. The CAR of paragraph 94, wherein the linker sequence comprises an amino acid sequence having at least 90% sequence identity to the amino acid sequence of SEQ ID NO: 41, 58, 61, 62, or 63.
96. The CAR of paragraph 94 or 95, wherein the linker sequence comprises the amino acid sequence of SEQ ID NO: 41, 58, 61, 62, or 63.
97. The CAR of any one of paragraphs 80-96, wherein the extracellular target-binding domain comprises an amino acid sequence having at least 90% sequence identity to the amino acid sequence of SEQ ID NO: 56 or 59.
98. The CAR of any one of paragraphs 80-97, wherein the extracellular target-binding domain comprises the amino acid sequence of SEQ ID NO: 56 or 59.
99. The CAR of any one of paragraphs 80-98, wherein the CAR comprises an amino acid sequence having at least 90% sequence identity to the amino acid sequence of SEQ ID NO: 39, 57, 64, or 65.
100. The CAR of any one of paragraphs 80-99, wherein the CAR comprises the amino acid sequence of SEQ ID NO: 39, 57, 64, or 65.
101. A CAR comprising an amino acid sequence having at least 90% sequence identity to the amino acid sequence of SEQ ID NO: 39, 57, 64, or 65.
102. A CAR comprising the amino acid sequence of SEQ ID NO: 39, 57, 64, or 65.
103. A polynucleotide encoding the CAR of any one of paragraphs 80-102.
104. The polynucleotide of paragraph 103, further comprising a suicide gene.
105. The polynucleotide of paragraph 103 or 104, further comprising a sequence encoding a signal sequence.
106. An immune cell comprising the CAR of any one of paragraphs 80-102 and/or the polynucleotide of any one of paragraphs 103-105.
107. The immune cell of paragraph 106, wherein the immune cell is a T or NK cell.
108. The immune cell of paragraph 106 or 107, wherein the immune cell is a human cell.
109. A pharmaceutical composition comprising the CAR of any one of paragraphs 80-102, the polynucleotide of any one of paragraphs 103-105, and/or the immune cell of any one of paragraphs 106-108.
110. A method of treating a cancer, a plasma cell disorder, an autoimmune disease or disorder, or transplant rejection in a subject, the method comprising administering to the subject the immune cell of any one of paragraphs 106-108 and/or the pharmaceutical composition of paragraph 109.
111. The method of paragraph 110, wherein the cancer comprises cells expressing BCMA and/or TACI.
112. The method of paragraph 110 or 111, wherein the cancer comprises cells with reduced BCMA expression.
113. The method of any one of paragraphs 110-112, wherein the subject is resistant to anti-BCMA therapy.
114. The method of any one of paragraphs 110-113, wherein the cancer is a myeloma or Waldenstrom's macroglobulinemia.
115. The method of paragraph 114, wherein the myeloma is multiple myeloma or smoldering myeloma.
116. The method of paragraph 110, wherein the plasma cell disorder is amyloidosis.
117. The method of paragraph 110, wherein the autoimmune disease or disorder is selected from the group consisting of hemophilia with antibodies to coagulation factors, myasthenia gravis, multiple sclerosis, and chronic graft versus host disease.
118. The method of paragraph 110, wherein the subject has high levels of anti-HLA antibodies.

Still other embodiments of the technology described herein can be defined according to any of the following additional numbered paragraphs:
1. A chimeric antigen receptor (CAR) polypeptide comprising:
   a) one or more extracellular domains comprising a portion of Tumor Necrosis Factor (TNF) superfamily receptor ligand;
   b) a hinge and transmembrane domain;
   c) a co-stimulatory domain; and
   d) an intracellular signaling domain.
2. The CAR polypeptide of paragraph 1, wherein the TNF superfamily receptor ligand is A Proliferation-Inducing Ligand (APRIL).
3. The CAR polypeptide of paragraph 1, wherein the TNF superfamily receptor ligand is TNF-alpha, lymphotoxin beta, OX40L, CD154, FasL, LIGHT, TL1A, CD70, Siva, CD153, 4-1BB ligand, TRAIL, RANKL, TWEAK, BAFF, CAMLG, LIGHT, NGF, BDNF, NT-3, NT-4, GITR ligand, TL1A, or EDA-A2.
4. The CAR polypeptide of any one of paragraphs 1-3, further comprising a CD8 leader sequence.
5. The CAR polypeptide of paragraph 4, wherein the CD8 leader sequence comprises the sequence selected from SEQ ID NO: 20, 26, or 32.
6. The CAR polypeptide of any of paragraphs 2, 4, or 5, wherein the portion of APRIL does not comprise a lysine-rich region of APRIL.
7. The CAR polypeptide of any of paragraphs 2 or 4-6, wherein the portion of APRIL comprises the sequence selected from SEQ ID NO: 21, 27, or 33.
8. The CAR polypeptide of any of paragraphs 1-7, wherein the hinge and transmembrane domain comprises the hinge and transmembrane domain of CD8 or 4-1 BB.
9. The CAR polypeptide of any of paragraphs 1-8, wherein the CD8 hinge and transmembrane domain sequence comprises the sequence of SEQ ID NO: 22.
10. The CAR polypeptide of any of paragraphs 1-9, wherein the 4-1 BB hinge and transmembrane domain sequence comprises the sequence selected from SEQ ID NO: 28 or 34.
11. The CAR polypeptide of any of paragraphs 1-10, wherein the intracellular signaling domain comprises the signaling domain of CD3ζ, CD3ε, or CD3θ.
12. The CAR polypeptide of any of paragraphs 1-11, wherein the CD3 intracellular signaling domain sequence comprises the sequence selected from SEQ ID NO: 24 or 30.
13. The CAR polypeptide of any of paragraphs 1-12, wherein the CD3θ intracellular signaling domain sequence comprises the sequence of SEQ ID NO: 36.
14. The CAR of any of paragraphs 1-13, wherein the co-stimulatory domain is the intracellular domain selected from the group consisting of 4-1 BB ICD, CD28 ICD, CD27 ICD, ICOS ICD, and OX40 ICD.
15. The CAR polypeptide of any of paragraphs 1-14, wherein the co-stimulatory domain is the intracellular domain of 4-1 BB.
16. The CAR polypeptide of paragraph 15, wherein the intracellular domain of 4-1 BB sequence comprises a sequence selected from SEQ ID NO: 23, 29, or 35.
17. The CAR polypeptide of any one of paragraphs 1-16, wherein the CAR polypeptide comprises two or more extracellular domains comprising a portion of TNF superfamily receptor ligand.
18. The CAR polypeptide of paragraph 17, wherein the CAR polypeptide comprises three extracellular domains comprising a portion of TNF superfamily receptor ligand.
19. A CAR polypeptide comprising at least 95% identity with a sequence selected from SEQ ID NO: 19, 25, or 31, or that is encoded by a sequence comprising at least 95% identity with a sequence selected from SEQ ID NO: 1, 7, or 13.
20. A CAR polypeptide comprising a sequence selected from SEQ ID NO: 19, 25, or 31, or that is encoded by a sequence selected from SEQ ID NO: 1, 7, or 13.
21. A CAR polypeptide comprising a sequence corresponding to a sequence selected from SEQ ID NO: 19, 25, or 31, or that is encoded by a sequence selected from SEQ ID NO: 1, 7, or 13.
22. A polypeptide complex comprising two or more of the CAR polypeptides of any one of paragraphs 1-21.
23. The polypeptide complex of paragraph 22, wherein the polypeptide complex comprises three CAR polypeptides of any one of paragraphs 1-21.
24. A mammalian cell comprising;
   a) a CAR polypeptide of any of paragraphs 1-21;
   b) a nucleic acid encoding a CAR polypeptide of any of paragraphs 1-21; or
   c) a polypeptide complex of paragraph 22 or 23.
25. The cell of paragraph 24, wherein the cell is a T cell.
26. The cell of paragraph 24 or 25, wherein the cell is a human cell.
27. The cell of any of paragraphs 24-26, wherein the cell is obtained from an individual having or diagnosed as having cancer, a plasma cell disorder, or autoimmune disease.
28. A method of treating cancer, a plasma cell disorder, amyloidosis, or an autoimmune disease in a subject, the method comprising:
a) engineering a T cell to comprise a CAR of any of paragraphs 1-21 on the T cell surface;
b) administering the engineered T cell to the subject.
29. A method of treating cancer, a plasma cell disorder, or an autoimmune disease in a subject, the method comprising administering a cell of any of paragraphs 24-27 to the subject.
30. The method of paragraph 28 or 29, wherein the cancer is BAFF+, BCMA+ and/or TACI⁺.
31. The method of any of paragraphs 28-30, wherein the subject is further administered an anti-BCMA therapy.
32. The method of any of paragraphs 28-31, wherein the subject is resistant to anti-BCMA therapies.
33. The method of any of paragraphs 28-32, wherein the cancer is multiple myeloma or smoldering myeloma.
34. The method of any of paragraphs 28-32, wherein the autoimmune disease is selected from the group consisting of hemophilia with antibodies to coagulation factors, myasthenia gravis, multiple sclerosis, and chronic graft v. host disease.
35. A composition comprising the CAR polypeptide of any one of paragraphs 1-21, the polypeptide complex of paragraph 22 or 23, or the cell of any one of paragraphs 24-27 formulated for the treatment of cancer.
36. The composition of paragraph 35, further comprising a pharmaceutically acceptable carrier.

### EXAMPLES

The following are examples of the methods and compositions of the invention. It is understood that various other embodiments may be practiced, given the description provided herein.

### Example 1. Design of APRIL-Based Chimeric Antigen Receptors (CARs)

Chimeric antigen receptors based on the extracellular domain of the APRIL (A PRoliferation-Inducing ligand) fused to transmembrane domains of CD8 or 4-1 BB and the signaling domain of the T cell activating receptors CD3 zeta, CD3 eta, or CD3 theta are described herein. These CARs can overcome resistance to anti-BCMA targeted therapies and utilize dimerizing and trimerizing transmembrane domains for optimal function. These CARs are contemplated for the treatment of cancer, e.g., multiple myelomas, plasma cell disorders, and/or severe autoimmune disease.

It was contemplated by the inventors that the natural ligand for BCMA could be used to engineer an antigen-binding moiety to generate anti-myeloma CAR T cells. CAR T cells based on scFvs and on the natural ligand (APRIL) were compared for cytotoxic activity, antigen-specific proliferation, and cytokine production in myeloma cell lines expressing BCMA, TACI, and/or BAFF-receptor.

BCMA is a small type-III transmembrane protein that binds BAFF with low affinity and APRIL with high affinity; BCMA signaling protects myeloma cells from apoptosis.

BCMA has two close family members: TACI and BAFF receptor. TACI is expressed at similar levels and stages of B cell development, whereas BAFF receptor is expressed in earlier stages of B cell development and has higher affinity for binding BAFF than APRIL. The intracellular domains of both BCMA and TACI interact with TRAFs, and likely have redundant functions in promoting plasma cell survival. Antibodies and scFvs raised specifically against BCMA are less likely to cross-react with TACI given the small epitope-binding regions of BCMA than vice versa. In fact, the literature indicates that none of the anti-BCMA products (antibodies, scFvs, or bi-specific T cell engagers) in the clinical setting cross-react with BAFF-receptor or TACI.

One of the greatest challenges in designing a CAR T cell with novel specificity is determining off-tumor expression of the target. Reassuringly, anti-BCMA products have been considered safe in a variety of clinical settings, without evidence of off-tumor reactivity. CAR T cell products directed to BCMA have been associated with cytokine release syndrome. However, publicly available data from TOGA, ENCODE, BLUEPRINT, and GTEX indicate that the expression profiles of BCMA and TACI appear to be safe for targeting via CAR T cells (data not shown); neither molecule is expressed by healthy adult tissues other than plasma cells and B cells, and both are expressed at high levels in multiple myeloma and chronic lymphocytic leukemia. Further, given emerging data regarding antigen-escape variants in patients with acute lymphoblastic leukemia receiving anti-CD19-directed CAR T cells, developing a re-directed T cell that binds two antigens with similar expression profiles and signaling redundancy can provide a mechanism of avoiding escape variants.

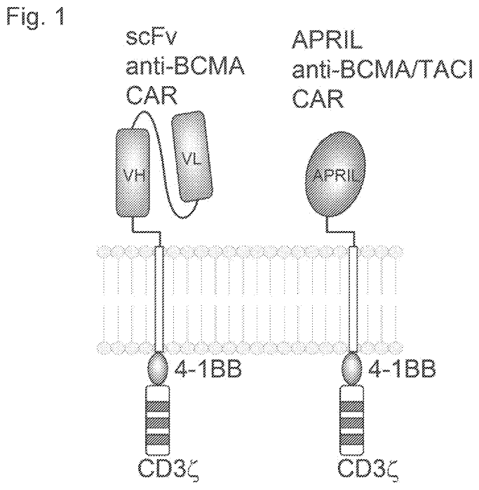

There are three putative ways to generate one CAR designed to react to two antigens. (1) Generate an scFv that cross-reacts with both targets. The danger with this strategy is that a promiscuous scFv may also have off-tumor reactivity that could be difficult to predict in the pre-clinical setting. (2) Generate a CAR composed of scFvs with two different specificities in tandem. This strategy is being pursued for CD19 and CD22 and for CD19 and CD20, for example. However, the optimal spacing between the two scFvs must be determined empirically, and formation of cross-reactive diabody-scFvs could also result in off-target binding. This method is feasible but challenging and expensive, especially since scFvs must be generated and tested independently, and then combined. (3) Develop a high-affinity ligand that binds to both receptors and fuse it to the remaining components of the chimeric antigen receptor (transmembrane and signaling domains). In this case, the inventors appreciated a unique opportunity to utilize the third approach with APRIL (FIG. 1).

There are four potential issues with respect to using APRIL as an extracellular binding domain for a CAR T cell: (1) APRIL naturally forms a homo-trimer, whereas scFv-based CARs are thought to homodimerize. It is not clear whether APRIL homodimers bind BCMA/TACI, or if CARs can signal if they form trimers. Of note, 4-1 BB also naturally forms a trimer, and yet CAR constructs that include a 4-1 BB costimulation domain are highly active, indicating flexibility of function between homodimerizing and homotrimerizing TNF-related proteins. Formation of active CARs with suitable binding to BCMA/TACI is easily tested in vitro via flow cytometry with soluble BCMA and TACI, as well as via cytotoxicity assays against target cells expressing BCMA and TACI. (2) APRIL also binds to heparan sulfate chains associated with proteoglycans of the syndecan family (including CD138, syndecan-1), which may have more disseminated expression than TACI and BCMA; thus, there is increased potential for off-tumor activity. Specifically, binding of APRIL to heparan sulfate chains occurs via the lysine-rich region in its N-terminus, whereas the TNF-like region interacts with the BCMA and TACI receptors. In myeloma cells, binding to CD138 can act as a co-receptor for APRIL binding to TACI. Due to the distance between putative binding of the APRIL CAR and the heparan sulfate proteoglycan molecules, it is not expected that this interaction will result in cytotoxicity, but this prediction can be tested systematically in cell lines expressing CD138 without TACI or BCMA. In addition, a form of APRIL that lacks the N-terminal lysine-rich region to avoid binding to heparan sulfate chains can be generated. (3) There is a putative receptor for APRIL, which has not been confirmed but is hypothesized to be expressed on epithelial tissue; this interaction would necessitate testing and modeling of APRIL-CAR directed activity against epithelial cells. (4) The natural APRIL sequence is cleaved from its endogenous transmembrane domain, and can promote survival signals in myeloma cells; it is therefore proposed to anchor only the N-terminus domains of APRIL (distal to the cleavage site) to the transmembrane and intracellular domains of the CAR, so as to avoid shedding APRIL from the CAR T cells.

Experimental Design

Described herein is the testing of a small panel of scFv sequences specific for BCMA based on published sequences of murine and phage-display derived anti-BCMA constructs in the context of our CAR backbone. In addition, an APRIL-based CAR, utilizing only the most extracellular portion of APRIL domains that bind to BCMA and TACI is characterized. Also described is an N-terminus-truncated version of APRIL to eliminate the lysine-rich region that binds to heparan sulfate chains. Next, lentiviral vectors with two scFv- and two APRIL-based CARs are used to test primary T cells for expression of the CAR via flow cytometry after staining with biotinylated soluble BCMA-Ig and TACI-Ig (commercially available). Finally, it is verified that APRIL-based CARs do not secrete or cleave APRIL as a soluble protein, by collecting supernatants from T cell cultures and measuring soluble APRIL via ELISA.

Target cell lines based on K562 cells were engineered to express BAFF-receptor, BCMA, and TACI singly and in combination via lentiviral transduction. K562 cells expressing CD138 (syndecan-1), are engineered to test for binding of APRIL-based CARs to this heparan sulfate proteoglycan. These lines provide targets and antigen-presenting cells in which to test anti-BCMA scFv-CARs and APRIL-CARs for their ability to lyse BCMA- and TACI-expressing targets, and undergo antigen-specific proliferation. CD138-bearing targets are tested for sensitivity to APRIL-CAR mediated binding and toxicity in the presence and absence of heparin (which eliminates binding between APRIL and heparan sulfate). Specific lysis is measured by co-culturing effector cells with target cells at various (E:T) ratios; target cells are also genetically modified to express luciferase, such that viable target cells can be quantified by measuring light emission.

The cross-reactivity of binding to the CARs is also measured by using soluble BCMA and soluble TACI as staining reagents for CAR T cells to be evaluated by flow cytometry. Anti-BCMA scFv-CAR T cells and APRIL-CAR T cells are tested for their ability to proliferate in an antigen-specific manner in response to targets presenting BCMA, TACI, or both. Proliferation is measured by dilution of the fluorescent dye CFSE, and by counting T cells over the course of one to two weeks following antigen stimulation.

Finally, primary human plasma cells from patients with multiple myeloma are examined for their expression of BCMA, TACI, and BAFF receptor by standard flow cytometry. The MGH myeloma group has a biobank of bone marrow specimens from patients with multiple myeloma, from which de-identified samples can be examined. The levels of BCMA, TACI, and BAFF-R in plasma cells from 30 patients with measurable plasma cell burden can be quantified. Where feasible, anti-BCMA and APRIL-based CAR T cells are co-cultured with viable primary myeloma plasma cells; co-cultures are evaluated for viability of the myeloma cells and proliferation of the CAR T cells. In addition, the levels of BCMA and TACI expression in the bone marrow plasma cells of patients who have received anti-BCMA scFv-based CAR T cells can be examined. In this case, BCMA and TACI expression can be quantified in baseline marrow samples and in a bone marrow sample that is collected at 1-3 months following treatment, or at relapse, in patients treated at our site.

It is expected that scFv-based and APRIL-based CAR-transduced primary T cells exert cytotoxic activity and proliferate in response to BCMA-expressing target cells, be they K562-transduced cell lines, myeloma cell lines such as U266 and RPMI-8226, or primary patient myeloma cells. In contrast, only APRIL-based CARs exert cytotoxic effects against cell lines expressing only TACI. APRIL-based CARs bind soluble versions of both TACI and BCMA, whereas scFv-based anti-BCMA CARs bind only to soluble BCMA.

Untransduced T cells and CD19-CAR transduced T cells are not expected to display cytotoxic activity in response to BCMA-expressing target cells or multiple myeloma cell lines; these cells serve as negative controls. APRIL-based CARs are not expected to secrete soluble APRIL into the culture medium; if detectable secretion occurs, as measured by ELISA or Luminex analysis of the supernatant, the CAR can be redesigned to an alternative format (based on an scFv that is cross-reactive between TACI and BCMA), or including fewer amino acid domains of the extracellular distal (C-terminus) portion of APRIL to further eliminate possible cleavage sites. APRIL-based and scFv-based anti-BCMA CARs are expected to yield similar levels of cytokine production, and proliferate similarly in response to BCMA-expressing targets, but only APRIL-based CARs are expected to produce IFNγ and IL-2 in response to TACI-expressing targets.

APRIL-based CARs are not expected to mediate cytolysis of CD138-expressing targets in the absence of TACI or BCMA due to the distance between binding sites; comparisons will be made to anti-CD138-scFv-based CARs, which have already been shown to eliminate myeloma cell lines in vitro and in vivo. However, if CD138-directed cytotoxicity is not observed with APRIL-based CARs, the heparan sulfate mechanism can be verified by adding heparin to abrogate this interaction. An N-terminus-truncated version of APRIL, so as to eliminate the lysine-rich region but maintain only the TNF-like region as the extracellular binding domain of the CAR, is also described herein. If there is any remaining question as to potential toxicity of APRIL-based CARs against heparan sulfate proteoglycans or epithelial tissues, cytotoxicity can be tested against primary cultured keratinocytes and in our skin-graft in vivo model. In this model, immunodeficient mice are grafted with human skin (discarded tissue from plastic surgery or circumcisions) and allowed to heal. Skin-toxicity of CAR T cells is monitored histopathologically from biopsies or graft excisions; skin toxicity is manifested as lymphocytic infiltration with destruction of the epidermal/dermal junction and keratinocyte apoptosis, which is the pathognomonic sign of graft-vs.-host disease. If there is remaining concern about possible epithelial toxicity of APRIL-based CAR T cells, safety of APRIL-based CARs can be evaluated in this model.

In bone marrow samples obtained from patients with multiple myeloma, it is expected to confirm high levels of expression of TACI and BCMA in plasma cells, with lower levels of BAFF-receptor as determined by flow cytometry and appropriate controls (fluorescence minus one).

### Example 2. Limiting Antigen Escape in Multiple Myeloma by Dual Antigen-Targeting

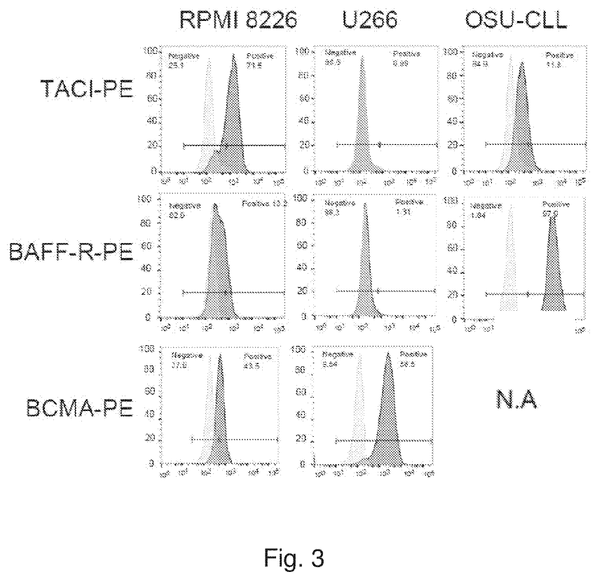

Despite recent advantages in treatment, multiple myeloma still remains an incurable disease. Several recent clinical trials of CAR T cells directed against B cell maturation antigen (BCMA) have lead to clinical responses including complete remission in patients with multiple myeloma. However, treatment failure due to antigen-loss of BCMA has already been described in some patients. The transmembrane activator and calcium modulator and cyclophilin ligand interactor (TACI) is thought to have a redundant role to BCMA in maintaining plasma cell survival, and is also highly expressed on multiple myeloma cells. In the work described herein, the natural ligand for BCMA and TACI, APRIL, was utilized as a CAR binding moiety. The approach prevents disease relapse due to antigen-escape by dual targeting of multiple surface antigens in multiple myeloma (FIG. 3).

Materials and Methods

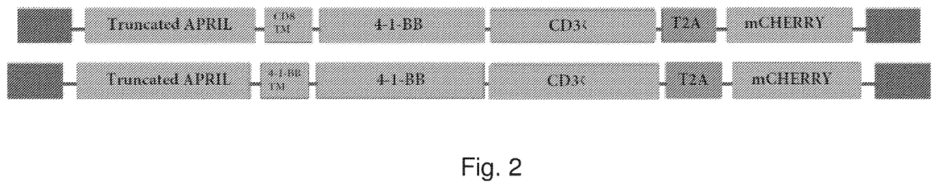

CAR constructs were generated with scFv-based anti-BCMA, and APRIL-based CARs bearing different hinge and transmembrane domains (CD8 or 4-1 BB), all fused to 4-1 BB and CD3 zeta (FIG. 2). Human primary T cells were lentivirally transduced with either an anti-BCMA-CAR or APRIL-based CARs. Cytotoxicity, proliferation and cytokine production was evaluated in vitro against a panel of cell lines with varying expression levels of BCMA and TACI and in vivo in a xenograft model of multiple myeloma.

Results

Increased activation in response to BCMA+ or TACI+ target cells, were seen for APRIL-based CARs. Anti-BCMA-CAR was only activated in response to BCMA+ target cells. Both BCMA and APRIL-CD8 hinge/transmembrane CARs displayed antigen-specific cytotoxicity. Interestingly, lower levels were found in cytokine production for APRIL-CD8 hinge/transmembrane CAR compared to anti-BCMA-CAR. This observation is likely to reflect the difference in binding affinity between using APRIL or an scFv as CAR binding moiety. Altering the hinge/transmembrane domain to 4-1 BB in the APRIL-CAR lead to a reduction in cytotoxicity and limited cytokine production. Ongoing studies, using a xenograft model have shown complete tumor remission in some mice treated with anti-BCMA-CAR or APRIL-CD8 hinge/transmembrane CAR.

Discussion

Described herein is the design of a CAR, based on the natural ligand APRIL, able to recognize both BCMA and TACI in order to limit potential antigen-escape in multiple myeloma. Inclusion of the CD8 hinge and transmembrane region was optimal for APRIL CAR function. Despite the cytotoxic efficacy of the APRIL CAR against tumor cells, lower levels of effector cytokine production were seen. This is an important finding, since CAR T cell therapy can lead to cytokine release syndrome.

### Example 3. T Cells Expressing APRIL-Based CARs

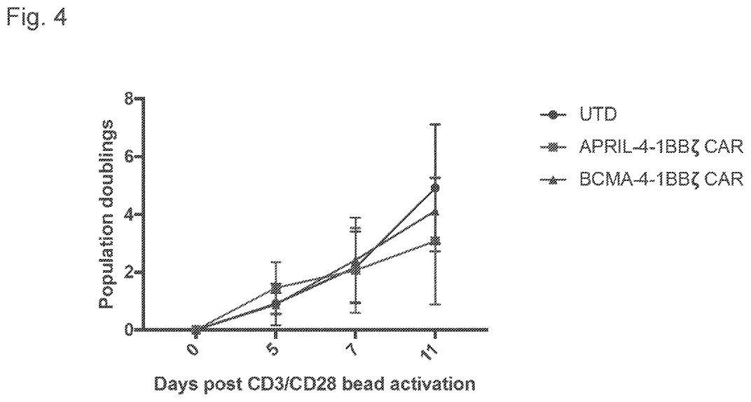

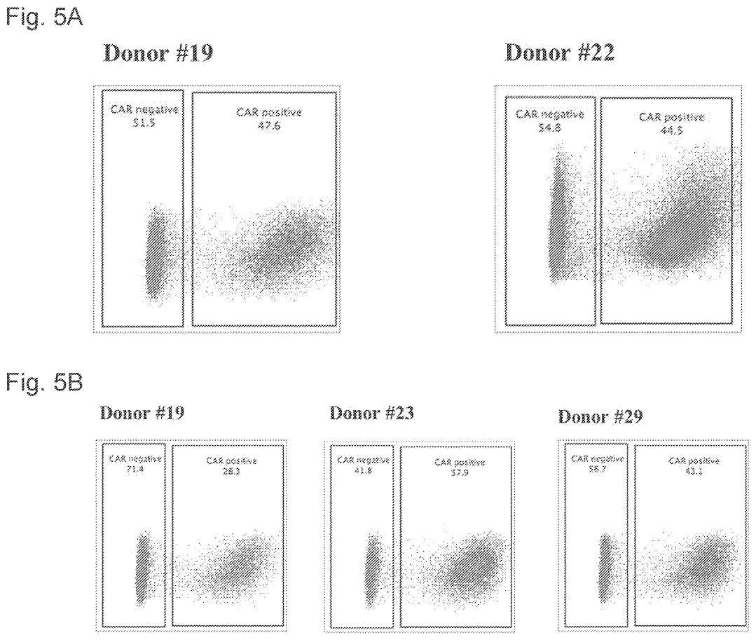

Human T cells were stimulated with CD3/28 beads on day 0 and transduced with lentiviral vector coding for APRIL-CD8TM-4-1 BBz CAR expressed APRIL-CD8TM-4-1BK CAR or BCMA-CD8TM-4-1BBζ CAR. Cells were counted beginning on day 0 and their growth was plotted as population doublings (FIG. 4). Transduction efficiency was measured by mCherry (reporter) positivity (FIGS. 5A-5B).

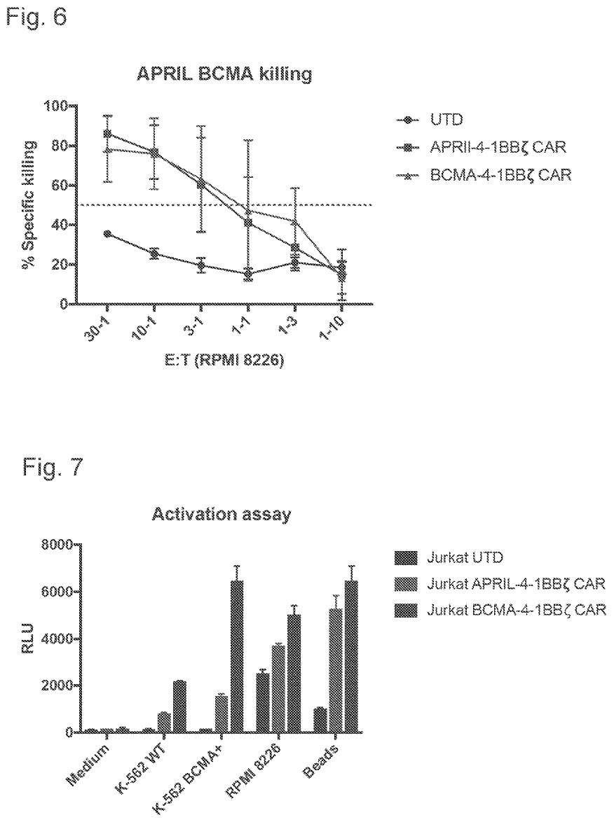

CAR-transduced T cells were incubated for 18 hours with target BCMA+ TACI+ multiple cells (RMPI-8226) that had been transduced to express luciferase. Specific lysis of target cell was calculated at the indicated effector:target ratios (FIG. 6).

CAR-mediated T cell activation was tested in a Jurkat cell line expressing luciferase behind the NFAT promoter (FIG. 7).

### Example 4. In Vitro Efficacy of APRIL CAR T Cells

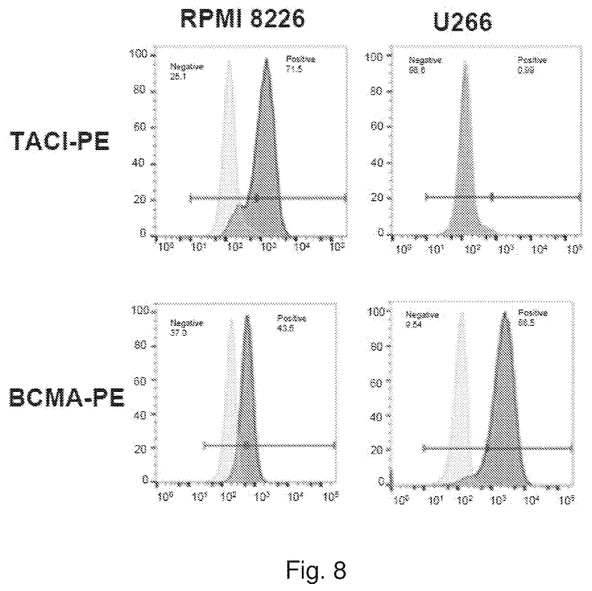

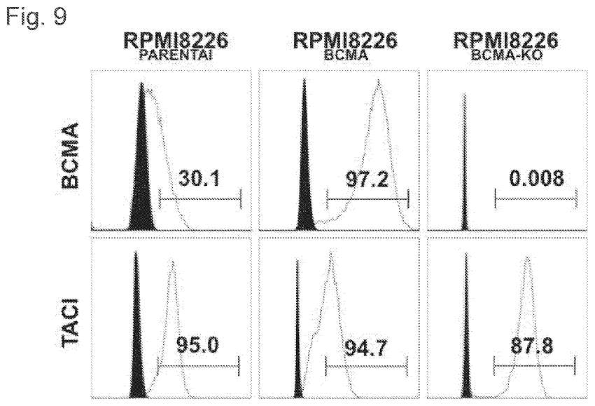

Surface expression of BCMA and TACI was measured in multiple myeloma cell lines (FIG. 8), and RPMI8226 was engineered to express various levels of BCMA (FIG. 9). TACI was transduced in to the RPMI-BCMA KO.

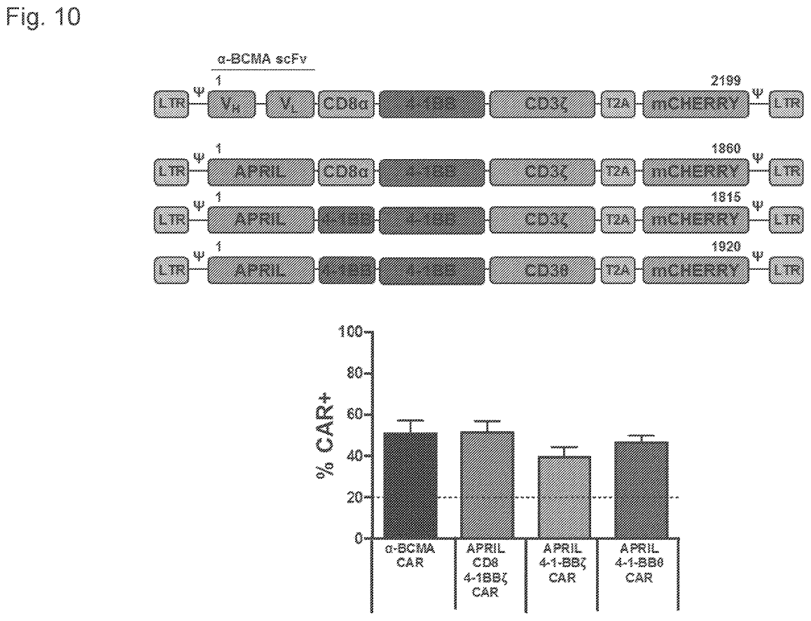

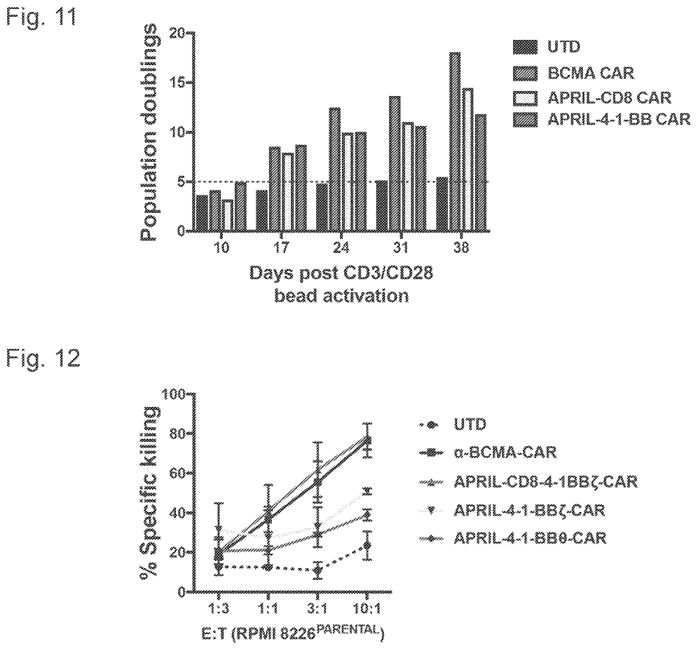

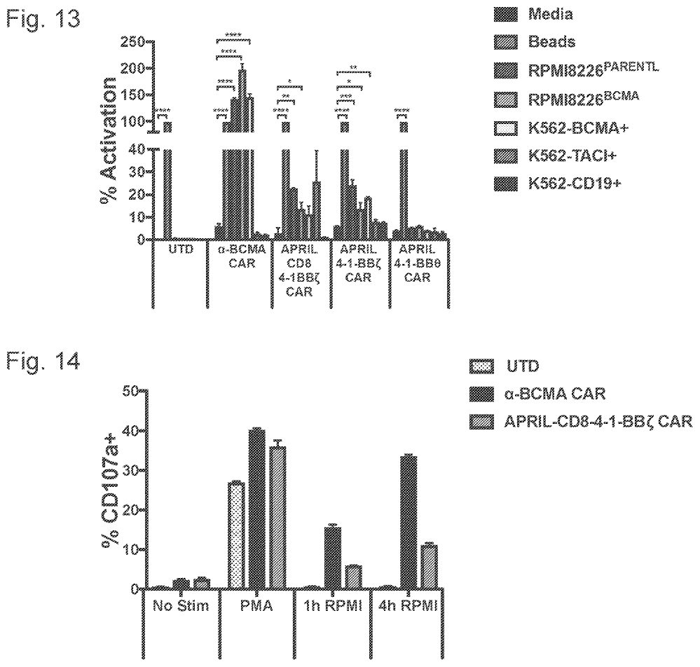

A number of APRIL and BCMA CAR constructs were designed and demonstrated to effectively transduce T cells (FIG. 10). T cells expressing the CARs expanded upon stimulation with BCMA-expressing cells (FIG. 11). APRIL-CAR expressing T cells demonstrated specific killing of cells expressing BCMA and TACI (FIG. 12) and activation was similarly specific (FIG. 13).

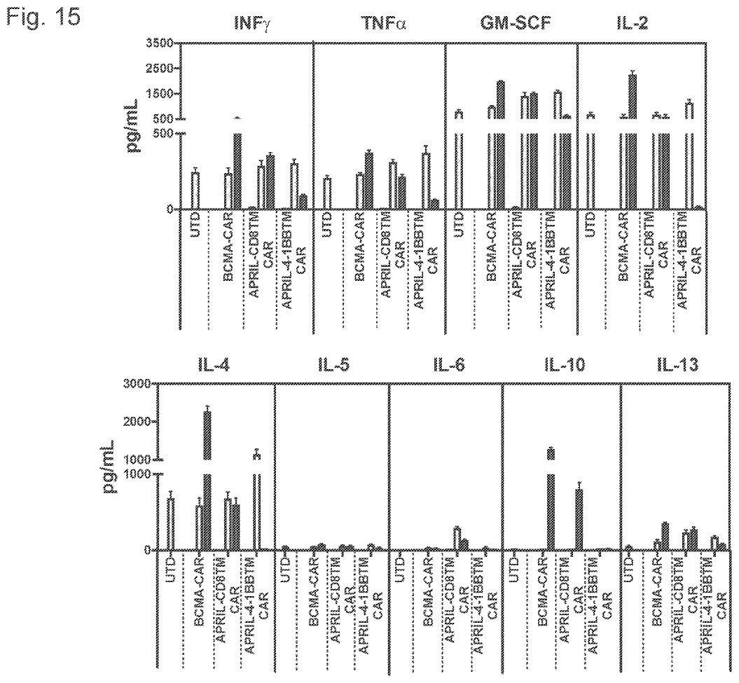

BCMA and APRIL CARs degranulate in response to stimulation with RPMI8226^{PARENTAL} (FIG. 14). The cytokine profile of APRIL CARs is depicted in FIG. 15

### Example 5. APRIL CAR Nucleic Acid Sequences

pMGH71 (APRIL-CD8 CAR)-
APRIL/CD8TM/4-1BB/CD3ζ (SEQ ID NO: 1) comprises:
CD8 leader (nucleotides 1-63 (SEQ ID NO: 2));
APRIL sequence (nucleotides 64-471 (SEQ ID NO: 3));
CD8 hinge and TM sequence (nucleotides 472-678 (SEQ ID NO: 4));
4-1BB ICD sequence (nucleotides 679-804 (SEQ ID NO: 5));
and CD3 zeta sequence (nucleotides 805-1140 (SEQ ID NO: 6)).
(SEQ ID NO: 1)
ATGGCCCTCCCTGTCACCGCCCTGCTGCTTCCGCTGGCTCTTCTGCTCCACGCCGCTCGGCCC
CACTCTGTCCTGCACCTGGTTCCCATTAACGCCACCTCCAAGGATGACTCCGATGTGACAGAGG
TGATGTGGCAACCAGCTCTTAGGCGTGGGAGAGGCCTACAGGCCCAAGGATATGGTGTCCGAA
TCCAGGATGCTGGAGTTTATCTGCTGTATAGCCAGGTCCTGTTTCAAGACGTGACTTTCACCATG
GGTCAGGTGGTGTCTCGAGAAGGCCAAGGAAGGCAGGAGACTCTATTCCGATGTATAAGAAGTA
TGCCCTCCCACCCGGACCGGGCCTACAACAGCTGCTATAGCGCAGGTGTCTTCCATTTACACCA
AGGGGATATTCTGAGTGTCATAATTCCCCGGGCAAGGGCGAAACTTAACCTCTCTCCACATGGA
ACCTTCCTGGGGTTTGTGAAACTGACCACTACCCCAGCACCGAGGCCACCCACCCCGGCTCCT
ACCATCGCCTCCCAGCCTCTGTCCCTGCGTCCGGAGGCATGTAGACCCGCAGCTGGTGGGGCC
GTGCATACCCGGGGTCTTGACTTCGCCTGCGATATCTACATTTGGGCCCCTCTGGCTGGTACTT
GCGGGGTCCTGCTGCTTTCACTCGTGATCACTCTTTACTGTAAGCGCGGTCGGAAGAAGCTGCT
GTACATCTTTAAGCAACCCTTCATGAGGCCTGTGCAGACTACTCAAGAGGAGGACGGCTGTTCA
TGCCGGTTCCCAGAGGAGGAGGAAGGCGGCTGCGAACTGCGCGTGAAATTCAGCCGCAGCGC
AGATGCTCCAGCCTACCAACAGGGGCAGAACCAGCTCTACAACGAACTCAATCTTGGTCGGAGA
GAGGAGTACGACGTGCTGGACAAGCGGAGAGGACGGGACCCAGAAATGGGCGGGAAGCCGCG
CAGAAAGAATCCCCAAGAGGGCCTGTACAACGAGCTCCAAAAGGATAAGATGGCAGAAGCCTAT
AGCGAGATTGGTATGAAAGGGGAACGCAGAAGAGGCAAAGGCCACGACGGACTGTACCAGGGA
CTCAGCACCGCCACCAAGGACACCTATGACGCTCTTCACATGCAGGCCCTGCCGCCTCGG
CD8 leader (SEQ ID NO: 2 (nucleotides 1-63 of SEQ ID NO: 1))
(SEQ ID NO: 2)
ATGGCCCTCCCTGTCACCGCCCTGCTGCTTCCGCTGGCTCTTCTGCTCCACGCCGCTCGGCCC
APRIL sequence SEQ ID NO: 3 (nucleotides 64-471 of SEQ ID NO: 1)
(SEQ ID NO: 3)
CACTCTGTCCTGCACCTGGTTCCCATTAACGCCACCTCCAAGGATGACTCCGATGTGACAGAGG
TGATGTGGCAACCAGCTCTTAGGCGTGGGAGAGGCCTACAGGCCCAAGGATATGGTGTCCGAA
TCCAGGATGCTGGAGTTTATCTGCTGTATAGCCAGGTCCTGTTTCAAGACGTGACTTTCACCATG
GGTCAGGTGGTGTCTCGAGAAGGCCAAGGAAGGCAGGAGACTCTATTCCGATGTATAAGAAGTA
TGCCCTCCCACCCGGACCGGGCCTACAACAGCTGCTATAGCGCAGGTGTCTTCCATTTACACCA
AGGGGATATTCTGAGTGTCATAATTCCCCGGGCAAGGGCGAAACTTAACCTCTCTCCACATGGA
ACCTTCCTGGGGTTTGTGAAACTG
CD8 hinge and TM sequence (SEQ ID NO: 4 (nucleotides 472-678 of SEQ ID NO: 1))
(SEQ ID NO: 4)
ACCACTACCCCAGCACCGAGGCCACCCACCCCGGCTCCTACCATCGCCTCCCAGCCTCTGTCC
CTGCGTCCGGAGGCATGTAGACCCGCAGCTGGTGGGGCCGTGCATACCCGGGGTCTTGACTTC
GCCTGCGATATCTACATTTGGGCCCCTCTGGCTGGTACTTGCGGGGTCCTGCTGCTTTCACTCG
TGATCACTCTTTACTGT
4-1BB ICD sequence (SEQ ID NO: 5 (nucleotides 679-804 of SEQ ID NO: 1))
(SEQ ID NO: 5)
AAGCGCGGTCGGAAGAAGCTGCTGTACATCTTTAAGCAACCCTTCATGAGGCCTGTGCAGACTA
CTCAAGAGGAGGACGGCTGTTCATGCCGGTTCCCAGAGGAGGAGGAAGGCGGCTGCGAACTG
CD3 zeta sequence (SEQ ID NO: 6 (nucleotides 805-1140 of SEQ ID NO: 1))
(SEQ ID NO: 6)
CGCGTGAAATTCAGCCGCAGCGCAGATGCTCCAGCCTACCAACAGGGGCAGAACCAGCTCTAC
AACGAACTCAATCTTGGTCGGAGAGAGGAGTACGACGTGCTGGACAAGCGGAGAGGACGGGAC
CCAGAAATGGGCGGGAAGCCGCGCAGAAAGAATCCCCAAGAGGGCCTGTACAACGAGCTCCAA
AAGGATAAGATGGCAGAAGCCTATAGCGAGATTGGTATGAAAGGGGAACGCAGAAGAGGCAAA
GGCCACGACGGACTGTACCAGGGACTCAGCACCGCCACCAAGGACACCTATGACGCTCTTCAC
ATGCAGGCCCTGCCGCCTCGG
pMGH76 (APRIL-4-1BB CAR)-
APRIL/4-1BBTM/4-1BB/CD3ζ (SEQ ID NO: 7) comprises
CD8 leader (nucleotides 1-63 (SEQ ID NO: 8));
APRIL sequence (nucleotides 64-471 (SEQ ID NO: 9));
4-1BB hinge and TM sequence (nucleotides 472-633 (SEQ ID NO: 10));
4-1BB ICD sequence (nucleotides 634-759 (SEQ ID NO: 11));
CD3 zeta sequence (nucleotides 760-1095 (SEQ ID NO: 12)).
(SEQ ID NO: 7)
ATGGCCCTCCCTGTCACCGCCCTGCTGCTTCCGCTGGCTCTTCTGCTCCACGCCGCTCGGCCC
CACTCTGTCCTGCACCTGGTTCCCATTAACGCCACCTCCAAGGATGACTCCGATGTGACAGAGG
TGATGTGGCAACCAGCTCTTAGGCGTGGGAGAGGCCTACAGGCCCAAGGATATGGTGTCCGAA
TCCAGGATGCTGGAGTTTATCTGCTGTATAGCCAGGTCCTGTTTCAAGACGTGACTTTCACCATG
GGTCAGGTGGTGTCTCGAGAAGGCCAAGGAAGGCAGGAGACTCTATTCCGATGTATAAGAAGTA
TGCCCTCCCACCCGGACCGGGCCTACAACAGCTGCTATAGCGCAGGTGTCTTCCATTTACACCA
AGGGGATATTCTGAGTGTCATAATTCCCCGGGCAAGGGCGAAACTTAACCTCTCTCCACATGGA
ACCTTCCTGGGGTTTGTGAAACTGCCATCTCCAGCCGACCTCTCTCCGGGAGCATCCTCTGTGA
CCCCGCCTGCCCCTGCGAGAGAGCCAGGACACTCTCCGCAGATCATCTCCTTCTTTCTTGCGCT
GACGTCGACTGCGTTGCTCTTCCTGCTGTTCTTCCTCACGCTCCGTTTCTCTGTTGTTAAGCGCG
GTCGGAAGAAGCTGCTGTACATCTTTAAGCAACCCTTCATGAGGCCTGTGCAGACTACTCAAGA
GGAGGACGGCTGTTCATGCCGGTTCCCAGAGGAGGAGGAAGGCGGCTGCGAACTGCGCGTGA
AATTCAGCCGCAGCGCAGATGCTCCAGCCTACCAACAGGGGCAGAACCAGCTCTACAACGAAC
TCAATCTTGGTCGGAGAGAGGAGTACGACGTGCTGGACAAGCGGAGAGGACGGGACCCAGAAA
TGGGCGGGAAGCCGCGCAGAAAGAATCCCCAAGAGGGCCTGTACAACGAGCTCCAAAAGGATA
AGATGGCAGAAGCCTATAGCGAGATTGGTATGAAAGGGGAACGCAGAAGAGGCAAAGGCCACG
ACGGACTGTACCAGGGACTCAGCACCGCCACCAAGGACACCTATGACGCTCTTCACATGCAGG
CCCTGCCGCCTCGG
CD8 leader sequence (SEQ ID NO: 8 (nucleotides 1-63 of SEQ ID NO: 7))
(SEQ ID NO: 8)
ATGGCCCTCCCTGTCACCGCCCTGCTGCTTCCGCTGGCTCTTCTGCTCCACGCCGCTCGGCCC
APRIL sequence (SEQ ID NO: 9 (nucleotides 64-471 of SEQ ID NO: 7))
(SEQ ID NO: 9)
CACTCTGTCCTGCACCTGGTTCCCATTAACGCCACCTCCAAGGATGACTCCGATGTGACAGAGG
TGATGTGGCAACCAGCTCTTAGGCGTGGGAGAGGCCTACAGGCCCAAGGATATGGTGTCCGAA
TCCAGGATGCTGGAGTTTATCTGCTGTATAGCCAGGTCCTGTTTCAAGACGTGACTTTCACCATG
GGTCAGGTGGTGTCTCGAGAAGGCCAAGGAAGGCAGGAGACTCTATTCCGATGTATAAGAAGTA
TGCCCTCCCACCCGGACCGGGCCTACAACAGCTGCTATAGCGCAGGTGTCTTCCATTTACACCA
AGGGGATATTCTGAGTGTCATAATTCCCCGGGCAAGGGCGAAACTTAACCTCTCTCCACATGGA
ACCTTCCTGGGGTTTGTGAAACTG
4-1BB hinge and TM sequence (SEQ ID NO: 10 (nucleotides 472-633 of SEQ ID NO: 7))
(SEQ ID NO: 10)
CCATCTCCAGCCGACCTCTCTCCGGGAGCATCCTCTGTGACCCCGCCTGCCCCTGCGAGAGAG
CCAGGACACTCTCCGCAGATCATCTCCTTCTTTCTTGCGCTGACGTCGACTGCGTTGCTCTTCCT
GCTGTTCTTCCTCACGCTCCGTTTCTCTGTTGTT
4-1BB ICD sequence (SEQ ID NO: 11 (nucleotides 634-759 of SEQ ID NO: 7))
(SEQ ID NO: 11)
AAGCGCGGTCGGAAGAAGCTGCTGTACATCTTTAAGCAACCCTTCATGAGGCCTGTGCAGACTA
CTCAAGAGGAGGACGGCTGTTCATGCCGGTTCCCAGAGGAGGAGGAAGGCGGCTGCGAACTG
CD3 zeta sequence (SEQ ID NO: 12 (nucleotides 760-1095 of SEQ ID NO: 7))
(SEQ ID NO: 12)
CGCGTGAAATTCAGCCGCAGCGCAGATGCTCCAGCCTACCAACAGGGGCAGAACCAGCTCTAC
AACGAACTCAATCTTGGTCGGAGAGAGGAGTACGACGTGCTGGACAAGCGGAGAGGACGGGAC
CCAGAAATGGGCGGGAAGCCGCGCAGAAAGAATCCCCAAGAGGGCCTGTACAACGAGCTCCAA
AAGGATAAGATGGCAGAAGCCTATAGCGAGATTGGTATGAAAGGGGAACGCAGAAGAGGCAAA
GGCCACGACGGACTGTACCAGGGACTCAGCACCGCCACCAAGGACACCTATGACGCTCTTCAC
ATGCAGGCCCTGCCGCCTCGG
pMGH77 (APRIL-4-1BBθ CAR)-
APRIL/4-1BBTM/4-1BB/CD3theta (SEQ ID NO: 13) comprising
CD8 leader (nucleotides 1-63 (SEQ ID NO: 14));
APRIL sequence (nucleotides 64-471 (SEQ ID NO: 15));
4-1BB hinge and TM sequence (nucleotides 472-633 (SEQ ID NO: 16));
4-1BB ICD sequence (nucleotides 634-759 (SEQ ID NO: 17));
CD3 theta sequence (nucleotides 760-1200) (SEQ ID NO: 18)).
(SEQ ID NO: 13)
ATGGCCCTCCCTGTCACCGCCCTGCTGCTTCCGCTGGCTCTTCTGCTCCACGCCGCTCGGCCC
CACTCTGTCCTGCACCTGGTTCCCATTAACGCCACCTCCAAGGATGACTCCGATGTGACAGAGG
TGATGTGGCAACCAGCTCTTAGGCGTGGGAGAGGCCTACAGGCCCAAGGATATGGTGTCCGAA
TCCAGGATGCTGGAGTTTATCTGCTGTATAGCCAGGTCCTGTTTCAAGACGTGACTTTCACCATG
GGTCAGGTGGTGTCTCGAGAAGGCCAAGGAAGGCAGGAGACTCTATTCCGATGTATAAGAAGTA
TGCCCTCCCACCCGGACCGGGCCTACAACAGCTGCTATAGCGCAGGTGTCTTCCATTTACACCA
AGGGGATATTCTGAGTGTCATAATTCCCCGGGCAAGGGCGAAACTTAACCTCTCTCCACATGGA
ACCTTCCTGGGGTTTGTGAAACTGCCATCTCCAGCCGACCTCTCTCCGGGAGCATCCTCTGTGA
CCCCGCCTGCCCCTGCGAGAGAGCCAGGACACTCTCCGCAGATCATCTCCTTCTTTCTTGCGCT
GACGTCGACTGCGTTGCTCTTCCTGCTGTTCTTCCTCACGCTCCGTTTCTCTGTTGTTAAGCGCG
GTCGGAAGAAGCTGCTGTACATCTTTAAGCAACCCTTCATGAGGCCTGTGCAGACTACTCAAGA
GGAGGACGGCTGTTCATGCCGGTTCCCAGAGGAGGAGGAAGGCGGCTGCGAACTGCGCGTGA
AATTCAGCCGCAGCGCAGATGCTCCAGCCTACCAACAGGGGCAGAACCAGCTCTACAACGAAC
TCAATCTTGGTCGGAGAGAGGAGTACGACGTGCTGGACAAGCGGAGAGGACGGGACCCAGAAA
TGGGCGGGAAGCCGCGCAGAAAGAATCCCCAAGAGGGCCTGTACAACGAGCTCCAAAAGGATA
AGATGGCAGAAGCCTATAGCGAGATTGGTATGAAAGGGGAACGCAGAAGAGGCAAAGGCCACG
ACGGACTGTACCAGGACAGCCACTTCCAAGCAGTTCCAGTACAGGAAAAGAAAAAAAGGCTCAG
AAGGGCACCGTGGCGTGCATTCGCCCAGCCCCAGAGGTTAAAGCACCGAAACAATGAACTACC
TGACTCCCTAGAGCCCATATATAAAAACATTTGGAACAAAACATTTATAGGAGAG
CD8 leader sequence (SEQ ID NO: 14 (nucleotides 1-63 of SEQ ID NO: 13))
(SEQ ID NO: 14)
ATGGCCCTCCCTGTCACCGCCCTGCTGCTTCCGCTGGCTCTTCTGCTCCACGCCGCTCGGCCC
APRIL sequence (SEQ ID NO: 15 (nucleotides 64-471 of SEQ ID NO: 13))
(SEQ ID NO: 15)
CACTCTGTCCTGCACCTGGTTCCCATTAACGCCACCTCCAAGGATGACTCCGATGTGACAGAGG
TGATGTGGCAACCAGCTCTTAGGCGTGGGAGAGGCCTACAGGCCCAAGGATATGGTGTCCGAA
TCCAGGATGCTGGAGTTTATCTGCTGTATAGCCAGGTCCTGTTTCAAGACGTGACTTTCACCATG
GGTCAGGTGGTGTCTCGAGAAGGCCAAGGAAGGCAGGAGACTCTATTCCGATGTATAAGAAGTA
TGCCCTCCCACCCGGACCGGGCCTACAACAGCTGCTATAGCGCAGGTGTCTTCCATTTACACCA
AGGGGATATTCTGAGTGTCATAATTCCCCGGGCAAGGGCGAAACTTAACCTCTCTCCACATGGA
ACCTTCCTGGGGTTTGTGAAACTG
4-1BB hinge and TM sequence (SEQ ID NO: 16 (nucleotides 472-633 of SEQ ID NO: 13))
(SEQ ID NO: 16)
CCATCTCCAGCCGACCTCTCTCCGGGAGCATCCTCTGTGACCCCGCCTGCCCCTGCGAGAGAG
CCAGGACACTCTCCGCAGATCATCTCCTTCTTTCTTGCGCTGACGTCGACTGCGTTGCTCTTCCT
GCTGTTCTTCCTCACGCTCCGTTTCTCTGTTGTT
4-1BB ICD sequence (SEQ ID NO: 17 (nucleotides 634-759 SEQ ID NO: 13))
(SEQ ID NO: 17)
AAGCGCGGTCGGAAGAAGCTGCTGTACATCTTTAAGCAACCCTTCATGAGGCCTGTGCAGACTA
CTCAAGAGGAGGACGGCTGTTCATGCCGGTTCCCAGAGGAGGAGGAAGGCGGCTGCGAACTG
CD3 theta sequence (SEQ ID NO: 18 (nucleotides 760-1200 of SEQ ID NO: 13))
(SEQ ID NO: 18)
CGCGTGAAATTCAGCCGCAGCGCAGATGCTCCAGCCTACCAACAGGGGCAGAACCAGCTCTAC
AACGAACTCAATCTTGGTCGGAGAGAGGAGTACGACGTGCTGGACAAGCGGAGAGGACGGGAC
CCAGAAATGGGCGGGAAGCCGCGCAGAAAGAATCCCCAAGAGGGCCTGTACAACGAGCTCCAA
AAGGATAAGATGGCAGAAGCCTATAGCGAGATTGGTATGAAAGGGGAACGCAGAAGAGGCAAA
GGCCACGACGGACTGTACCAGGACAGCCACTTCCAAGCAGTTCCAGTACAGGAAAAGAAAAAAA
GGCTCAGAAGGGCACCGTGGCGTGCATTCGCCCAGCCCCAGAGGTTAAAGCACCGAAACAATG
AACTACCTGACTCCCTAGAGCCCATATATAAAAACATTTGGAACAAAACATTTATAGGAGAG

### Example 6. APRIL CAR Amino Acid Sequences

In one embodiment, as described elsewhere herein, specific residues are involved in binding to BCMA/TACI, namely: D132, T175, D205, R206, R231 of APRIL. The location of those residues are depicted below with bold type.

pMGH71 (APRIL-CD8 CAR)-
CD8Leader/APRIL/CD8hinge + TM/4-1BB/CD3z (SEQ ID NO: 19) comprising
CD8 leader (amino acids 1-21 (SEQ ID NO: 20));
APRIL sequence (amino acids 22-157 (SEQ ID NO: 21));
CD8 hinge and TM sequence (amino acids 158-226 (SEQ ID NO: 22));
4-1BB ICD sequence (amino acids 227-268 (SEQ ID NO: 23));
CD3 zeta sequence (amino acids 269-380) (SEQ ID NO: 24)).
(SEQ ID NO: 19)
MALPVTALLLPLALLLHAARPHSVLHLVPINATSKDDSDVTEVMWQPALRRGRGLQAQGYGVRIQDA
GVYLLYSQVLFQDVTFTMGQVVSREGQGRQETLFRCIRSMPSHPDRAYNSCYSAGVFHLHQGDILS
VIIPRARAKLNLSPHGTFLGFVKLTTTPAPRPPTPAPTIASQPLSLRPEACRPAAGGAVHTRGLDFACD
IYIWAPLAGTCGVLLLSLVITLYCKRGRKKLLYIFKQPFMRPVQTTQEEDGCSCRFPEEEEGGCELRV
KFSRSADAPAYQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR
CD8 leader sequence (SEQ ID NO: 20 (amino acids 1-21 of SEQ ID NO: 19))
(SEQ ID NO: 20)
MALPVTALLLPLALLLHAARP
APRIL sequence (SEQ ID NO: 21 (amino acids 22-157 of SEQ ID NO: 19))
(SEQ ID NO: 21)
HSVLHLVPINATSKDDSDVTEVMWQPALRRGRGLQAQGYGVRIQDAGVYLLYSQVLFQDVTFTMGQ
VVSREGQGRQETLFRCIRSMPSHPDRAYNSCYSAGVFHLHQGDILSVIIPRARAKLNLSPHGTFLGFV
KL
CD8 hinge and TM sequence (SEQ ID NO: 22 (amino acids 158-226 of SEQ ID NO: 19))
(SEQ ID NO: 22)
TTTPAPRPPTPAPTIASQPLSLRPEACRPAAGGAVHTRGLDFACDIYIWAPLAGTCGVLLLSLVITLYC
4-1BB ICD sequence (SEQ ID NO: 23 (amino acids 227-268 of SEQ ID NO: 19))
(SEQ ID NO: 23)
KRGRKKLLYIFKQPFMRPVQTTQEEDGCSCRFPEEEEGGCEL
CD3 zeta sequence (SEQ ID NO: 24 (amino acids 269-380 of SEQ ID NO: 19))
(SEQ ID NO: 24)
RVKFSRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
pMGH76 (APRIL-4-1BB CAR)-
CD8Leader/APRIL/4-1BBhinge + TM/4-1BB/CD3z (SEQ ID NO: 25) comprises
CD8 leader (amino acids 1-21 (SEQ ID NO: 26));
APRIL sequence (amino acids 22-157 (SEQ ID NO: 27));
4-1BB hinge and TM sequence (amino acids 158-211 (SEQ ID NO: 28));
4-1BB ICD sequence (amino acids 212-253 (SEQ ID NO: 29));
CD3 zeta sequence (amino acids 254-365 (SEQ ID NO: 30)).
(SEQ ID NO: 25)
MALPVTALLLPLALLLHAARPHSVLHLVPINATSKDDSDVTEVMWQPALRRGRGLQAQGYGVRIQDA
GVYLLYSQVLFQDVTFTMGQVVSREGQGRQETLFRCIRSMPSHPDRAYNSCYSAGVFHLHQGDILS
VIIPRARAKLNLSPHGTFLGFVKLPSPADLSPGASSVTPPAPAREPGHSPQIISFFLALTSTALLFLLFFL
TLRFSVVKRGRKKLLYIFKQPFMRPVQTTQEEDGCSCRFPEEEEGGCELRVKFSRSADAPAYQQGQ
NQLYNELNLGRREEYDVLDKRRGRDPEMGGKPRRKNPQEGLYNELQKDKMAEAYSEIGMKGERRR
GKGHDGLYQGLSTATKDTYDALHMQALPPR
CD8 leader sequence (SEQ ID NO: 26 (amino acids 1-21 of SEQ ID NO: 25))
(SEQ ID NO: 26)
MALPVTALLLPLALLLHAARP
APRIL sequence (SEQ ID NO: 27 (amino acids 22-157 of SEQ ID NO: 25))
(SEQ ID NO: 27)
HSVLHLVPINATSKDDSDVTEVMWQPALRRGRGLQAQGYGVRIQDAGVYLLYSQVLFQDVTFTMGQ
VVSREGQGRQETLFRCIRSMPSHPDRAYNSCYSAGVFHLHQGDILSVIIPRARAKLNLSPHGTFLGFV
KL
4-1BB hinge and TM sequence (SEQ ID NO: 28 (amino acids 158-211 of SEQ ID NO: 25))
(SEQ ID NO: 28)
PSPADLSPGASSVTPPAPAREPGHSPQIISFFLALTSTALLFLLFFLTLRFSVV
4-1BB ICD sequence (SEQ ID NO: 29 (amino acids 212-253 of SEQ ID NO: 25))
(SEQ ID NO: 29)
KRGRKKLLYIFKQPFMRPVQTTQEEDGCSCRFPEEEEGGCEL
CD3 zeta sequence (SEQ ID NO: 30 (amino acids 254-365 of SEQ ID NO: 25)
(SEQ ID NO: 30)
RVKFSRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
pMGH77 (APRIL-4-1BBθ CAR)-
CD8Leader/APRIL/4-1BBhinge + TM/4-1BB/CD3theta (SEQ ID NO: 31)
comprises CD8 leader (amino acids 1-21 (SEQ ID NO: 32));
APRIL sequence (amino acids 22-157 (SEQ ID NO: 33));
4-1BB hinge and TM sequence (amino acids 158-211 (SEQ ID NO: 34));
4-1BB ICD sequence (amino acids 212-253 (SEQ ID NO: 35));
CD3 theta sequence (amino acids 254-400) (SEQ ID NO: 36)).
(SEQ ID NO: 31)
MALPVTALLLPLALLLHAARPHSVLHLVPINATSKDDSDVTEVMWQPALRRGRGLQAQGYGVRIQDA
GVYLLYSQVLFQDVTFTMGQVVSREGQGRQETLFRCIRSMPSHPDRAYNSCYSAGVFHLHQGDILS
VIIPRARAKLNLSPHGTFLGFVKLPSPADLSPGASSVTPPAPAREPGHSPQIISFFLALTSTALLFLLFFL
TLRFSVVKRGRKKLLYIFKQPFMRPVQTTQEEDGCSCRFPEEEEGGCELRVKFSRSADAPAYQQGQ
NQLYNELNLGRREEYDVLDKRRGRDPEMGGKPRRKNPQEGLYNELQKDKMAEAYSEIGMKGERRR
GKGHDGLYQDSHFQAVPVQEKKKRLRRAPWRAFAQPQRLKHRNNELPDSLEPIYKNIWNKTFIGE
CD8 leader sequence (SEQ ID NO: 32 (amino acids 1-21 of SEQ ID NO: 31))
(SEQ ID NO: 32)
MALPVTALLLPLALLLHAARP
APRIL sequence (SEQ ID NO: 33 (amino acids 22-157 of SEQ ID NO: 31))
(SEQ ID NO: 33)
HSVLHLVPINATSKDDSDVTEVMWQPALRRGRGLQAQGYGVRIQDAGVYLLYSQVLFQDVTFTMGQ
VVSREGQGRQETLFRCIRSMPSHPDRAYNSCYSAGVFHLHQGDILSVIIPRARAKLNLSPHGTFLGFV
KL
4-1BB hinge and TM sequence (SEQ ID NO: 34 (amino acids 158-211 of SEQ ID NO: 31))
(SEQ ID NO: 34)
PSPADLSPGASSVTPPAPAREPGHSPQIISFFLALTSTALLFLLFFLTLRFSVV
4-1BB ICD sequence (SEQ ID NO: 35 (amino acids 212-253 of SEQ ID NO: 31))
(SEQ ID NO: 35)
KRGRKKLLYIFKQPFMRPVQTTQEEDGCSCRFPEEEEGGCEL
CD3 theta sequence (SEQ ID NO: 36 (amino acids 254-400 of SEQ ID NO: 31))
(SEQ ID NO: 36)
RVKFSRSADAPAYQQGQNQLYNELNLGRREEYDVLDKRRGRDPEMGGKPRRKNPQEGLYNELQK
DKMAEAYSEIGMKGERRRGKGHDGLYQDSHFQAVPVQEKKKRLRRAPWRAFAQPQRLKHRNNEL
PDSLEPIYKNIWNKTFIGE

### Example 7. Ligand Oligomerization to Enhance CARs Targeting Multimeric Antigens

In the work described in this study, natural oligomerization (e.g., homotrimerization) was used to develop ligand-based CARs with increased activity against cells expressing their cognate receptor. Certain ligands for cell surface receptors, including ligands of the TNF superfamily, are known to oligomerize (e.g., trimerize) to bind their cognate receptor. For example, as described above, human myeloma is known to express two surface antigens that may be targeted for effective antitumor antigens: BCMA and TACI. BCMI and TACI share a common ligand, APRIL, which is a compact self-forming trimer which binds with nanomolar affinity to TACI and BCMA.

A homotrimeric APRIL CAR construct was designed and constructed (FIG. 16). This construct is referred to herein as “TriPRIL CAR” and includes three tandem APRIL polypeptides connected through linkers, a CD8 hinge/transmembrane domain (CD8 TM), a 4-1 BB intracellular domain (4-1 BB), and a CD3ζ intracellular domain (CD3). This construct is operably linked to a promoter (e.g., an EF1α promoter).

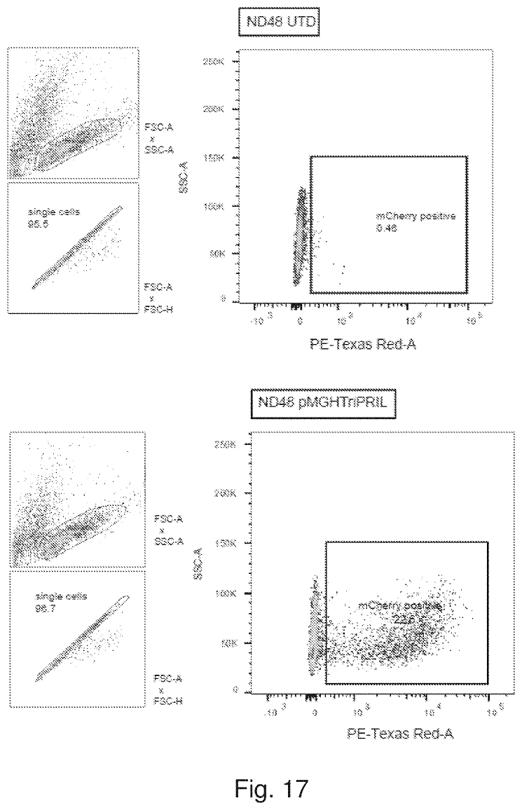

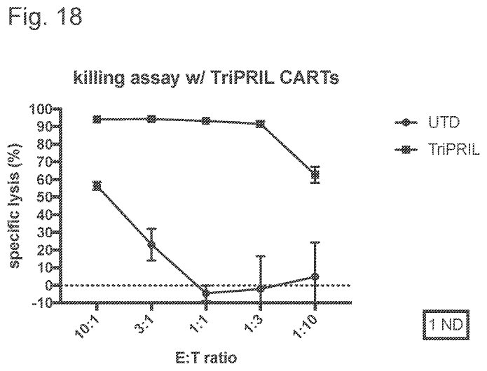

The transduction efficiency of the TriPRIL CAR construct into primary human T cells was evaluated (FIG. 17). Approximately 22.6% of the cells were mCherry-positive, compared to approximately 0.46% for the untransduced control. Therefore, the TriPRIL CAR construct can be transduced into primary human T cells. TriPRIL CAR expressing T cells demonstrated specific killing of cells expressing BCMA and TACI (FIG. 18). Therefore, TriPRIL CARs are useful therapeutic agents for treatment of tumors expressing BCMA, TACI, and/or BAFF-receptor, e.g., myeloma.

Analogous CAR constructs using other self-oligomerizing ligands (e.g., TNF superfamily ligands (e.g., TNF-alpha, lymphotoxin beta, OX40L, CD154, FasL, LIGHT, TL1A, CD70, Siva, CD153, 4-1BB ligand, TRAIL, RANKL, TWEAK, BAFF, CAMLG, LIGHT, NGF, BDNF, NT-3, NT-4, GITR ligand, TL1A, or EDA-A2)) can be used to target killing of unwanted cells expressing the cognate receptor, e.g., tumor cells.

### Example 8. APRIL-Based CAR T Cells for Multiple Myeloma

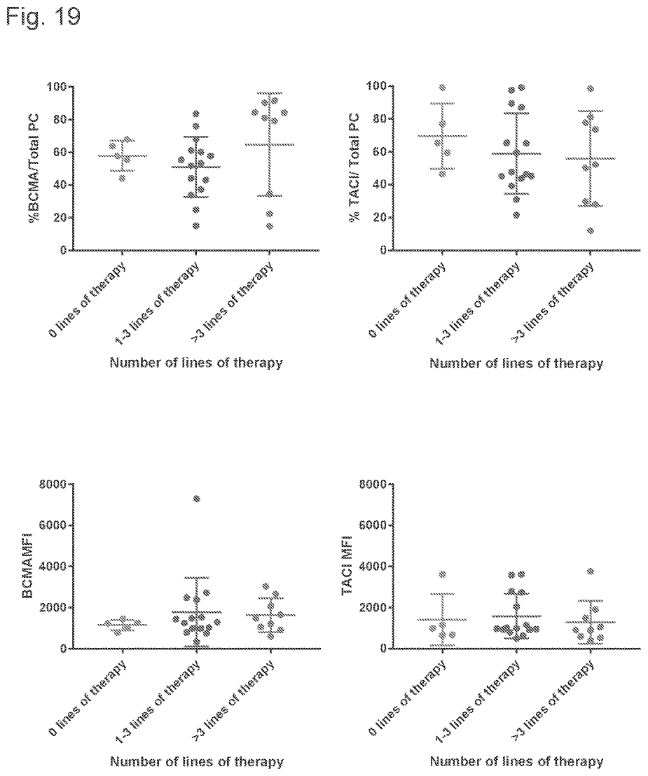

BCMA and TACI represent promising targets for treating, e.g., multiple myeloma, as BCMA and TACI are expressed on nearly all malignant plasma cells. BCMA and TACI expression as determined on plasma cells of multiple myeloma patients is shown in FIG. 19 (n=25).

As described herein, a CAR including three monomers of truncated APRIL fused together by short peptide linkers, referred to as TriPRIL CAR, was designed to target BCMA and TACI simultaneously.

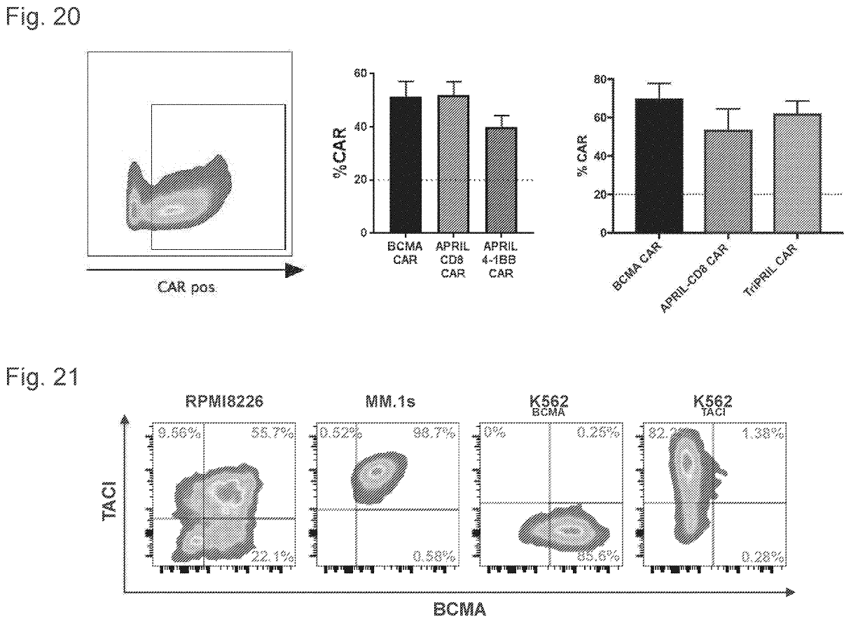

To this end, human T cells were activated with CD3/CD28 coated dyna beads and lentivirally transduced (MOI=5, n=3) to express BCMA CAR (pMGH8, FIG. 10), APRIL-CD8 CAR (pMGH71, FIG. 10), APRIL-4-1 BB CAR (pMGH76, FIG. 10), or TriPRIL CAR (pMGH71b, FIG. 16). The transduction efficiency of the CAR T cells was established based on mCherry expression, as shown in FIG. 20.

### Example 9. Degranulation and Activation of APRIL-Based CAR T Cells

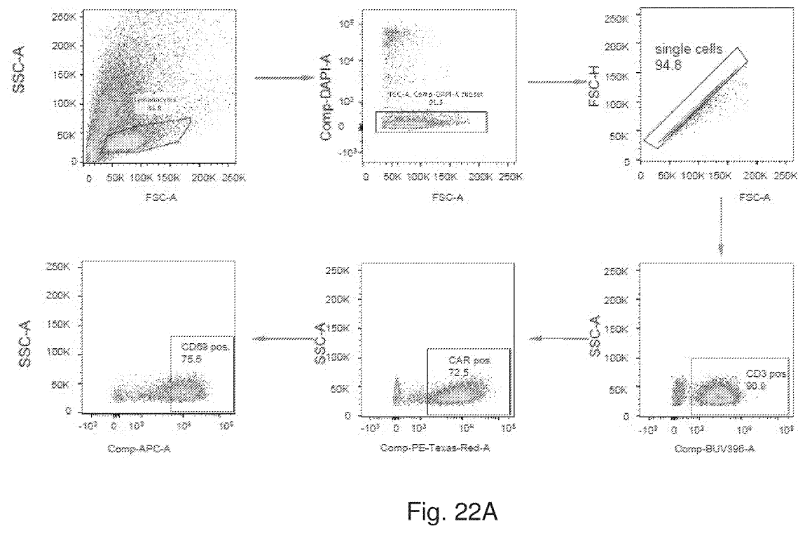

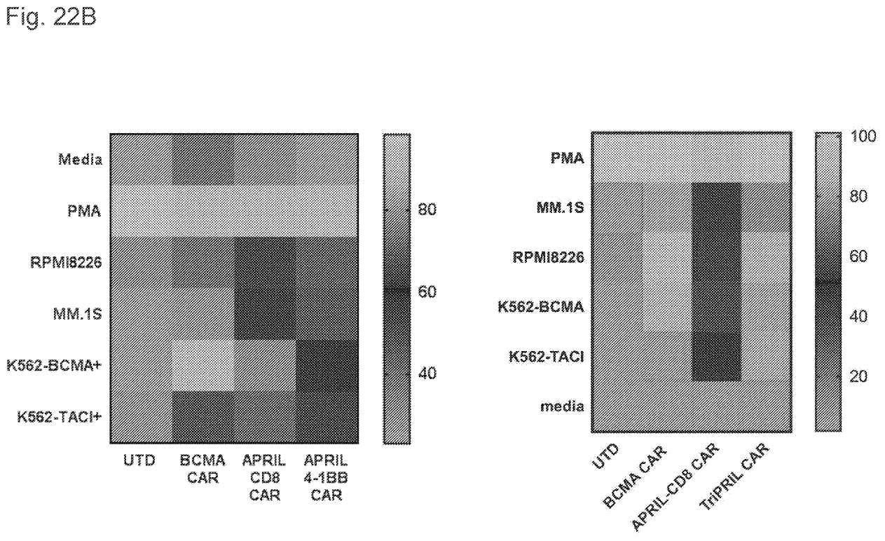

Expression of BCMA and TACI was determined for target cell lines RMP18226, MM.1 S, K562-BCMA, and K562-TACI (FIG. 21). The activation of the CAR T cells was measured in an in vitro CD69 activation assay (FIGS. 22A and 22B). BCMA CAR, APRIL-CD8 CAR, APRIL-4-1 BB CAR, and TriPRIL CAR T cells were assessed. UTD and CAR T cells from the same donors were incubated with MM.1S, RPMI8226, K562-BCMA and K562-TACI target cells for 12 hours. PMA stimulation and media served as positive and negative controls, respectively. Activation was measured based on the percent of CD69 expression on the UTD and CAR T cells (n=3).

### Example 10. Killing and Cytokine Production of APRIL-Based CART Cells In Vitro

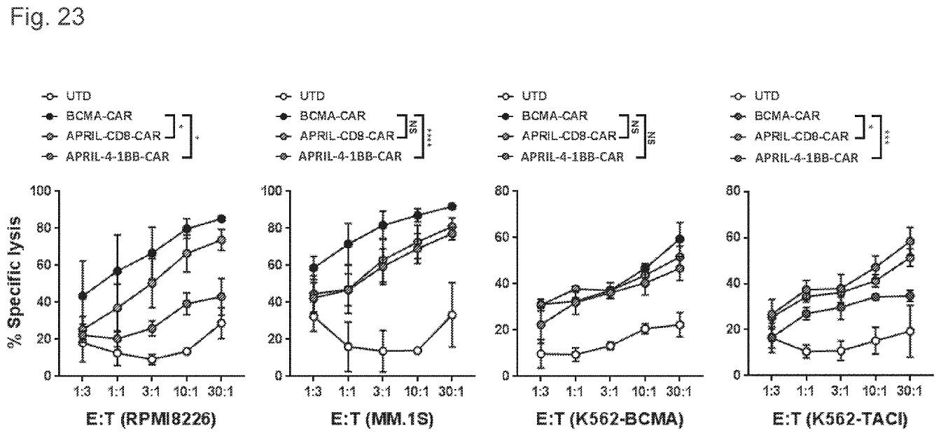

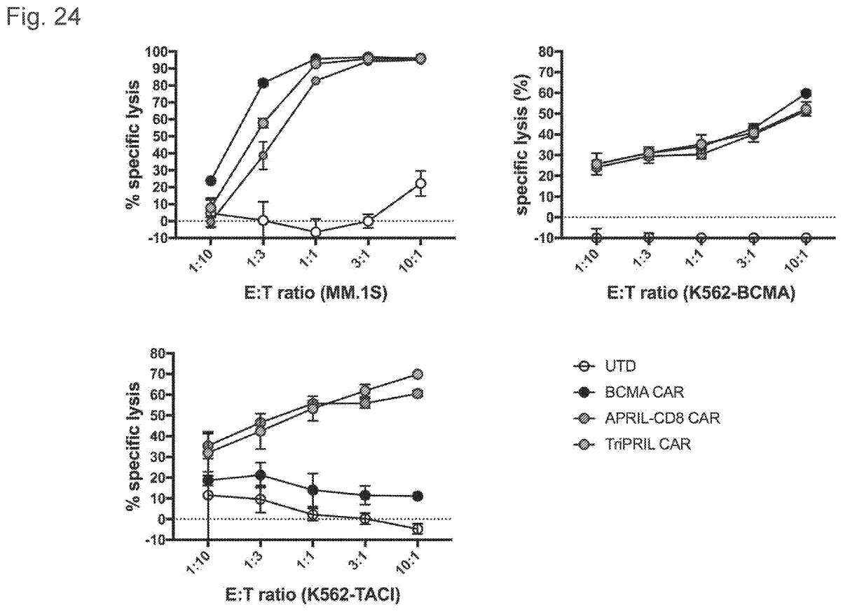

An in vitro killing assay was performed, where UTDs and CAR T cells from the same donor were incubated with RPMI8226, MM.1S, K562-BCMA and K562-TACI target cells (all CBG-GFP) for 16 hours at different ratios. Specific lysis was calculated based on bioluminescence measurement with the following equation: % specific lysis=100× (lum_{spontaneous death}−lumₜₑₛₜ)/lum_{spontaneous death}; where lum=luminescence. Mean and standard deviation were calculated form triplicate samples. The killing of UTD, BCMA CAR, APRIL-CD8 CAR, and APRIL-4-1 BB CAR T cells is shown in FIG. 23. Similarly, the killing for UTD, BCMA CAR, APRIL-CD8 CAR, and TriPRIL CAR T cells of MM.1 S, K562-BCMA, and K562-TACI target cells is shown in FIG. 24.

### Example 11. In Vivo Potency of APRIL CAR T Cells

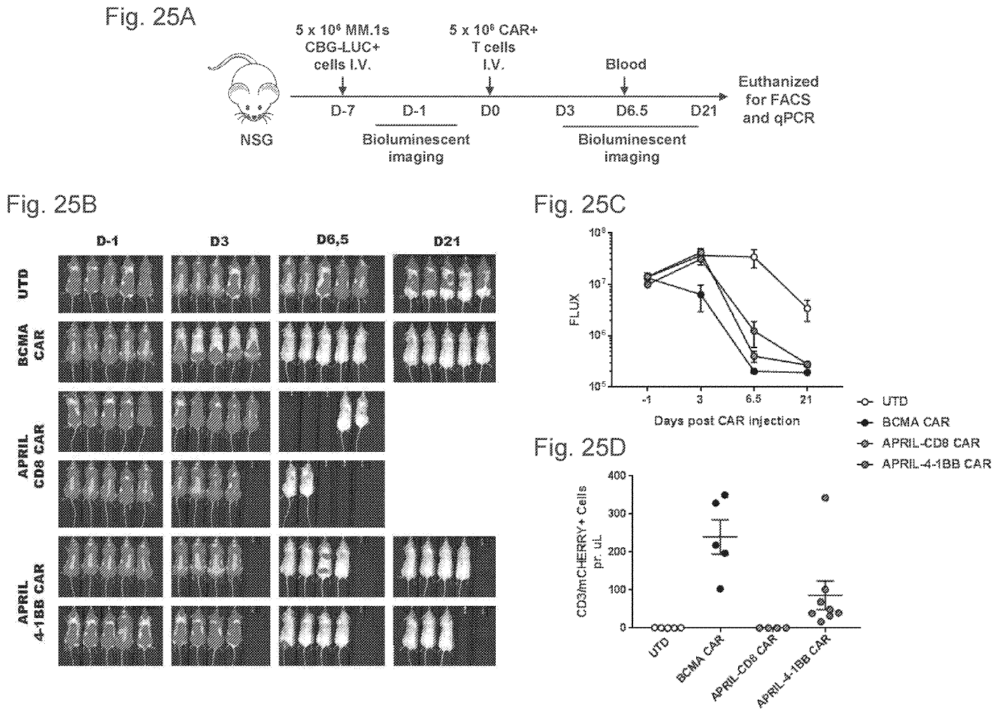

NOD scid gamma (NSG) mice were engrafted with 5×10⁶ luciferase-expressing MM.1 S cells. 7 days after tumor injection, mice were randomized according to tumor burden and received either 5×10⁶ UTD, BCMA-CAR, APRIL-CD8 or APRIL-4-1 BB CAR T cells (n=5-9 pr. group) (FIG. 25A). Bioluminescence images of mice receiving UTD, BCMA-CAR, APRIL-CD8 or APRIL-4-1 BB CAR T cells at days 1, 3, 6.5, and 21 post-CAR injection is shown in FIG. 25B, and the quantification of tumor burden in mice over time is shown in FIG. 25C. The absolute number of CD3+/mCherry positive cells was quantified in blood 6.5 days after injection of CARs or UTD using TrueCount beads (n=4-9 pr. group), which is shown in FIG. 25D.

### Example 12. Materials and Methods

Flow Cytometry of Primary Patient Plasma Cells

Additional stains for BCMA and TACI on 29 bone aspirate samples from patients carrying a diagnosis of multiple myeloma were performed, which were sent to the clinical flow cytometry laboratory; the additional stains were performed under an IRB-approved protocol. Plasma cells were gated based on forward and side scatter, dim for CD45, and positive for CD138 and CD38.

CAR Construct Design

All CARs were molecular designed at the amino acid level based on published sequences, and codon-optimized for mammalian expression using Geneious software. Nucleotide sequences were synthesized and cloned into a third-generation self-inactivating lentiviral vector transfer plasmid under the regulation of a human EF-1α promoter. Vectors also contained a second transgene coding for the fluorescent reporter mCherry to facilitate enumeration of transduction efficiency. Lentiviral vectors were generated using standard protocols.

Primary Human T Cell Culture and CAR T Cell Manufacturing

Primary human T cells were purified (Stem Cell Technologies, Cat #15061) from anonymous healthy donor leukopaks (purchased from the MGH blood bank) under an IND-approved protocol and cryopreserved. For expansions and subsequent assays, T cells were thawed and cultured in RPMI medium (10% fetal bovine serum (FBS), 1% penicillin/streptomycin (P/S), complemented with 20 IU/ml rhIL-2 at a cell concentration of 0.5-2×10⁶/ml. Human T cells were activated with anti-CD3/CD28 Dynabeads (Life Technologies) at a 1:3 ratio and for CAR T cell production a lentiviral vector was added 24 hours later at a multiplicity of infection of 5-10. At day 10-14 of culture CAR expression was measured and normalized for by adding in donor-matched activated untransduced T cells (UTDs) prior to cryopreservation. For in vitro and in vivo experiments CAR T cells were used immediately after thawing.

Cell Lines and Culture Conditions

The MM.1S, RPMI8226, K562, Jurkat and NK-92 cells were purchased form the American Type Culture Collection (ATCC). MM.1S BCMA KO cells were generated by first transducing MM.1S cells with Cas9-encoding lentiviral vectors and then electoporation with guide RNAs from the Brunello library (Doench et al., Nat. Biotechnol. 34(2):184-19 (2016)) and antibiotic selection. K562 cells were stably transduced with lentiviral vectors to express BCMA (K562-BCMA) or TACI (K562-TACI). Jurkat cells with a CD3ζ KO were transduced to stably express our CARs of interest. Where necessary, the cell lines were modified to constitutively express click beetle green luciferase (CBG-luc). To obtain pure cell populations post modification, the cells expressing the desired phenotype were sorted on a FACSAria (BD). RPMI media containing 10% FBS, 1% P/S (K562, Jurkat and NK-92 cells) or RPMI media containing 20% FBS, 1% P/S (MM.1S, RPMI8226) was used for propagation of the cell lines.

Flow Cytometry

Antibodies used: human BCMA-PE (clone 19F2, Biolegend), human CD3-BUV395 (clone UCHT1, BD), human CD3-FITC (clone SK7, BD), human CD4-AF647, human CD4-BV510 (clone SK3, BD), human CD4-BV786 (clone SK3, BD), human CD8-AF647, human CD8-APC H7 (clone SK1, BD), human CD8-BUV395 (clone RPA-T8, BD), human/mouse CD11b-APC (clone M1/70, Biolegend), human CD69-APC (clone FN50, Biolegend), human CD107a-AF700 (clone H4A3, BD), mouse Ly-6G/Ly-6C(Gr-1)-APC (clone RB6-8C5, Biolegend), mouse NK-1.1-APC (clone PK136, Biolegend), human TACI-PE (clone 1A1, Biolegend), human TACI-APC (clone 1A1, Biolegend), mouse TER-119/erythroid cells-APC (clone TER-119, Biolegend). Live/dead staining used: DAPI (Thermo Fisher Scientific, Cat P162247), Aqua (Thermo Fisher Scientific, Cat #L34965). Cells were stained at 4° C. in the dark for 30 minutes, washed twice in PBS+2% FBS and acquired on a Fortessa X-20 (BD).

To measure CAR binding affinity, soluble (s) BCMA and sTACI was conjugated to APC using Lightning-Link® APC antibody and protein labeling size (Innova Biosciences, Cat #SKU: 705-0010) according to manufacturer's protocol. Cells were washed in PBS+4% bovine serum albumin and incubated with the indicated amount of APC-conjugated sBCMA and sTACI at 4° C. in the dark for 45 minutes. After washing the cells three times mean fluorescence intensity (MFI) was measured.

Degranulation and activation of T cells was analyzed after co-culture with target cells at a 1:1 ration for 5 h in the presence of brefeldin A and CD107a antibody or overnight, respectively. Subsequently, cells were stained for CD3, CD4 and CD8 or CD3, CD4, CD8 and CD69 expression, respectively. Persistence of CAR T cells in the peripheral blood of treated mice was quantified using Trucount™ tubes (BD, Cat #340334) according to manufacturer's protocol. Cells were stained for human CD3, human/mouse CD11b, mouse Ly-6G/Ly-6C, mouse NK-1.1 and mouse TER-119 expression.

Additional Reagents

Additional reagents used include recombinant human APRIL (Peprotech, Cat #310-100), recombinant human BCMA (Peprotech, Cat #310-16), recombinant human IL-2 (Peprotech, Cat #200-02), recombinant human TACI (Peprotech, Cat #310-17), cell lysing solution 10× concentrate (Cat #349202, BD), cell stimulation cocktail (500×) containing PMA/ionomycin (eBioscience, Cat #00-4970-93), and protein transport inhibitor containing brefeldin A (GlogiPlug BD, Cat #555029).

Cellular Cytotoxicity, Bulk Cytokine Analysis, and Single-Cell 32-Plex IsoCode Chip Proteomics

For cytotoxicity assays, normalized CAR T cells were co-cultured with CBG-luc expressing target cells at indicated ratios for 8 hours (MM.1 S, RPMI8226) or overnight (K562-BCMA, K562-TACI). A luciferase assay system (Promega, Cat #E1501) was used according to manufacturer's protocol and luciferase activity was measured on a Synergy Neo2 luminescence microplate reader (Biotek). AH samples were measured in technical triplicates. The percent of specific lysis was calculated using the following formula: % specific lysis=(total RLU/target cells only RLU)×100 (RLU: relative luminescence unit).

Bulk cytokine analysis was performed on cell free supernatants from overnight co-cultures of indicated effector and target cells at a 1:1 ratio using a multiplex Luminex array (Luminex Corp., FLEXMAP 3D).

For single-cell 32-plex IsoCode chip proteomics CD4+ and CD8+ T cell subsets were separated using anti-CD4 or anti-CD8 microbeads (Miltenyi) and then stimulated with K562-BCMA and K562-TACI cells at a ratio of 1:1:1 for 20 hours. The untransduced T cells by the same stimulation were used as a negative control. The co-cultured CD4+ or CD8+ T cells were further enriched by the depletion of K562 cells by using anti-CD235a conjugated magnetic beads. CAR T cells were stained for CD4 and CD8 expression at room temperature for 10 minutes, rinsed once with phosphate-buffered saline (PBS), and resuspended in complete RPMI medium at a density of 1×10⁶/mL. Approximately 30 μl of cell suspension was loaded into the IsoCode Chip and incubated at 37° C., 5% CO₂ for additional 16 hours. Protein secretions from ˜1000 single T cells were captured by the 32-plex antibody barcoded chip and the polyfunctional profile was analyzed by the IsoSpeak software across the five functional groups:

Effector: Granzyme B, TNFα, IFN-γ, MIP1α, Perforin, TNFβ;

Stimulatory: GM-CSF, IL-2, IL-5, IL-7, IL-8, IL-9, IL-12, IL-15, IL-21;

Chemoattractive: CCL11, IP-10, MIP-1β, RNATES;

Regulatory; IL-4, IL-10, IL-13, IL-22, sCD137, sCD40L, TGFβ1;

Inflammatory: IL-6, IL-17A, IL-17F, MCP-1, MCP-4, IL-1β.

The PSI of CD4-F or CD8-F T cells was computed using a pre-specified formula, defined as the percentage of polyfunctional cells, multiplied by mean fluorescence intensity (MFI) of the proteins secreted by those cells:

$${PSI}_{sample} = ( \% polyfunctional cells in sample ) {\sum }_{i = 1}^{32} MFI of secreted protein i of the polyfunctional cells$$

The polyfunctional CD4+ or CD8+ T cell subsets that co-secreted combinatorial proteins at the single-cell level were further revealed by the polyfunctional heatmap.

Long-Term Proliferation Assay

CAR T cell proliferation in response to antigen stimulation was assessed by co-culturing normalized CAR T cells at a 1:1 ratio with BCMA- and TACI-expressing K562 cells that had previously been irradiated at 10,000 rads. T cells were counted and re-plated with irradiated target cells once weekly.

In Vivo Studies

All animal experiments were conducted under an MGH-approved protocol, in accordance with Federal and Institutional Animal Care and Use Committee (IACUC) requirements. For xenograft studies, NOD SCID γ-chain−/− mice (NSG, Jackson Laboratories) were injected intravenously (i.v.) with 1×10⁶ MM.1 S or MM.1S BCMA KO cells transduced to express CBG-luc. After engraftment of tumor was confirmed by bioluminescent imaging 14 days later, 2×10⁶ CAR T cells or UTDs were injected i.v. Tumor burden was monitored by bioluminescent imaging (BLI) and peripheral blood was collected and examined for CAR T cell persistence by flow cytometry. BLI was performed after intraperitoneal injection of D-luciferin substrate solution (30 mg/ml, Fischer Scientific, Cat #P188294) on an Ami spectral imaging apparatus and analyzed using IDL software version 4.3.1. Mice were euthanized as specified in the experimental protocol either when ending the experiment or when meeting IACUC pre-defined endpoints.

Statistical Analysis

Unless otherwise stated, a 2-tailed Student t test or 2-way ANOVA test was used for normal data at equal variance. Significance was considered for p<0.05, and is indicated by * in the legends. Analyses were performed with GraphPad Prism Version 7.0.

### Example 13. CARs Designed to Target BCMA and/or TACI

Targeting B cell maturation antigen (BCMA) with chimeric antigen receptor (CAR) T cells has shown great success in the treatment of multiple myeloma (MM), but is limited by heterogeneous antigen expression and imminent antigen escape of tumor cells. Combinatorial antigen targeting may help address these challenges. Taking the naturally occurring receptor-ligand pairs as a model, monomeric and trimeric A Proliferation-Inducing Ligand (APRIL) based CARs targeting BCMA and transmembrane activator and CAML interactor (TACI) simultaneously. BCMA and TACI are both only expressed on B cells and are upregulated on nearly all malignant plasma cells, making them attractive targets for multiple myeloma.

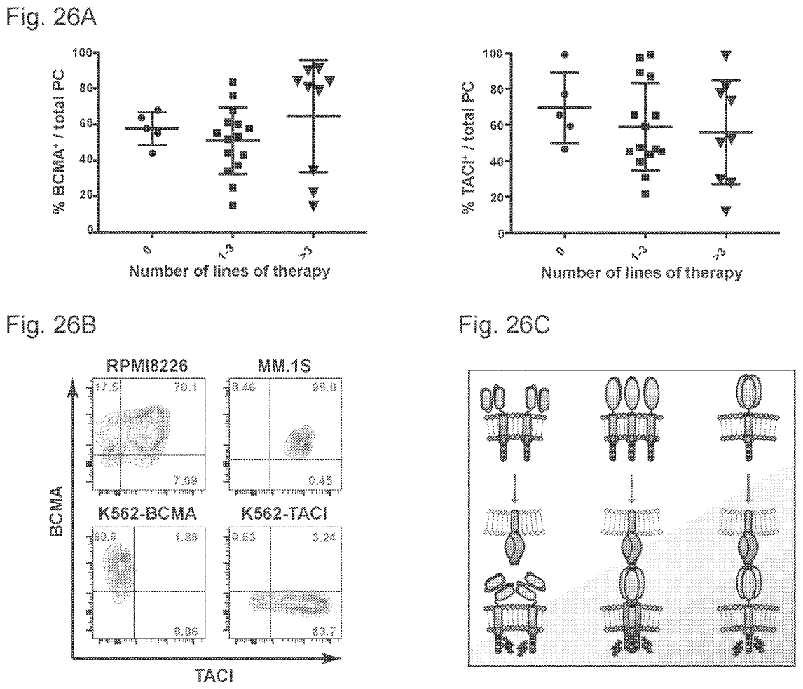

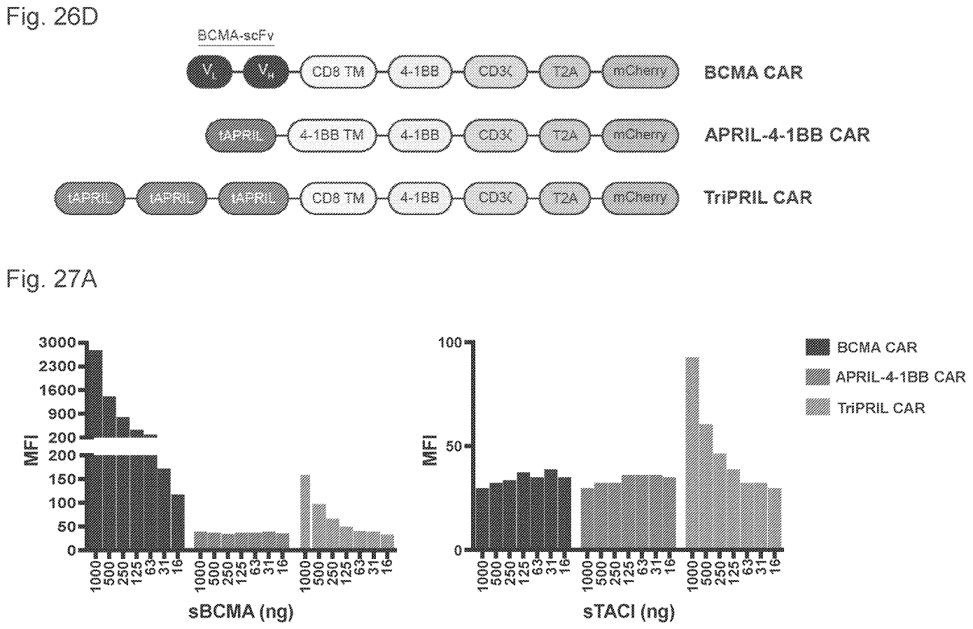

The surface expression of BCMA and TACI on plasma cells obtained from bone marrow samples of 29 MM patients was assessed. First, plasma cells were obtained from multiple myeloma patients and were stained with either BCMA-PE or TACI-PE to determine BCMA and TACI expression. Plasma cells were gated on forward and side scatter, CD45dim, CD38+ and CD138+. It was observed that the majority of plasma cells were positive for BCMA and TACI with similar distribution patterns. Expression of both BCMA and TACI were detected irrespective of the number of previous lines of therapy. At more advanced therapy stages (>3 previous lines of therapy) there was a tendency for BCMA to separate into either highly positive (>=80%) or slightly positive (20-30%) while TACI expression did not seem to change as much (FIG. 26A). Of note, the lower expression of BCMA in more heavily treated patients was not explained solely by prior treatment with BCMA-directed therapy, as only one patient in this cohort had previously received BCMA-directed treatment. Reflecting the expression patterns of BCMA and TACI in myeloma patients, the MM cell lines RPMI8226 and MM.1 S both express BCMA and TACI on their surface. In addition, K562 artificial antigen-presenting cells expressing either BCMA (K562-BCMA) or TACI (K562-TACI) were generated to facilitate testing of the CAR T cells. The levels of expression of BCMA and TACI on human multiple myeloma cell lines RMP18226 and MM.1S, K562 cells modified to express BCMA, and K562 cells modified to express TACI were determined, as shown in FIG. 26B.

To design CARs targeting both BCMA and TACI, the natural ligand APRIL was used to serve as a component of the extracellular target-binding domain. To avoid potential off-target interactions, a truncated version of APRIL, lacking the furin protease cleavage and the proteoglycan-binding sites, was employed as the binding moiety. Three different second-generation CAR constructs were designed: an anti-BCMA CAR based on an anti-BCMA scFv (BCMA CAR) serving as a control, a CAR containing one membrane-tethered truncated APRIL monomer (APRIL-4-1BB CAR), and a CAR with three truncated and fused APRIL monomers (TriPRIL CAR, e.g., SEQ ID NO: 39). Schematic diagrams of the three CARs designed is shown in FIG. 26D. The BCMA and TriPRIL CAR constructs have a CD8 transmembrane domain and 4-1 BB-CD3ζ intracellular domain, while the APRIL-4-1 BB CAR has a 4-1 BB transmembrane domain and 4-1 BB-CD3ζ intracellular domain. The constructs were cloned into lentiviral vectors and expressed under the control of an EF-1α promoter. In addition, all constructs contained an mCherry fluorescent reporter following a 2A element to allow for convenient flow-based detection of CAR expression. In vitro effector function was compared by cytotoxic potency, activation (CD69), degranulation (CD107a), cytokine production, and proliferation in response to target antigens. In vivo anti-tumor efficiency was assessed in a xenograft mouse model of MM.

APRIL is a TNF family member, and BCMA and TACI are TNFR family members; because TNF/TNFR interactions occur with trimeric forms of the molecules, it was hypothesized that binding and signaling of APRIL-based CARs would be enhanced by facilitating trimerization, either by linking a TNFR family member's transmembrane domain or by trimerizing only the APRIL moiety while maintaining a stable CAR configuration (FIG. 26C). CARs that use scFv-based binders and the CD8 transmembrane domain form homodimers (FIG. 26C), even when bearing 4-1 BB intracellular signaling domains (Imai et al., Leukemia. 18(4):676-84 (2004)). Truncated APRIL was therefore synthesized either as a monomer (APRIL-4-1 BB CAR) fused to a 4-1 BB transmembrane and intracellular domain, then CD3ζ intracellular domain in tandem, or as three APRIL monomers connected by linkers (TriPRIL CAR). A diagram depicting the theoretical binding of the three different CARs to BCMA is shown in FIG. 26C. The left panel shows binding of BCMA CAR, wherein CARs normally dimerize for signaling. The middle panel shows binding of APRIL-4-1 BB CAR, wherein APRIL naturally trimerizes to bind BCMA/TACI. The right panel shows binding of TriPRIL CAR, where the TriPRIL CAR includes trimerized APRIL as the extracellular target-binding domain. CAR T cell manufacturing of all three constructs was accomplished successfully (transduction efficiency 46-78%) from three different donors. Western blot analysis of CARs showed multimerized forms of the TriPRIL and BCMA CAR, while only the monomeric form of the APRIL CAR was detected.

### Example 14. Affinity and Effector Function of CAR T Cells

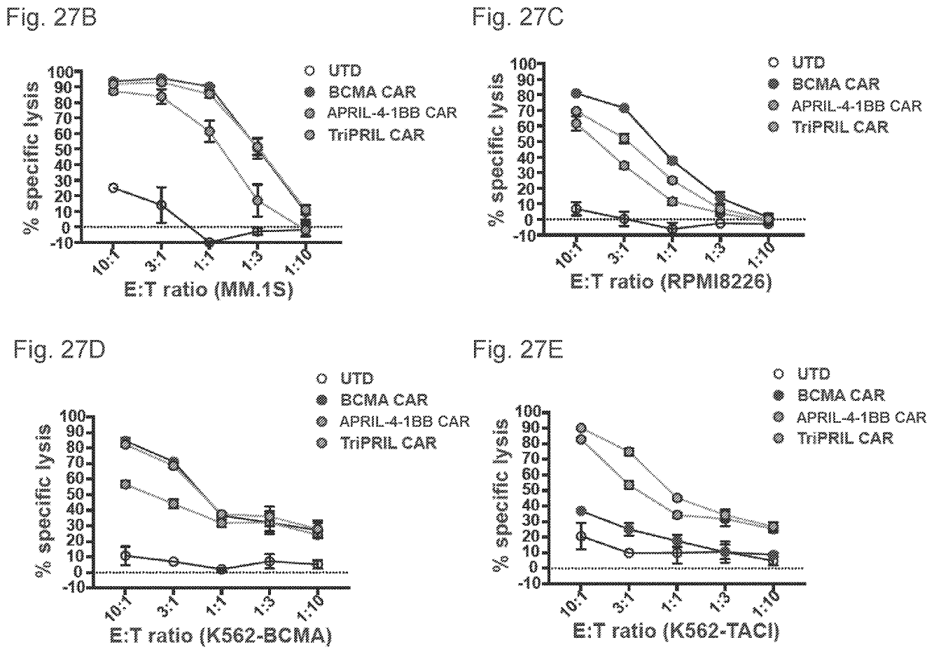

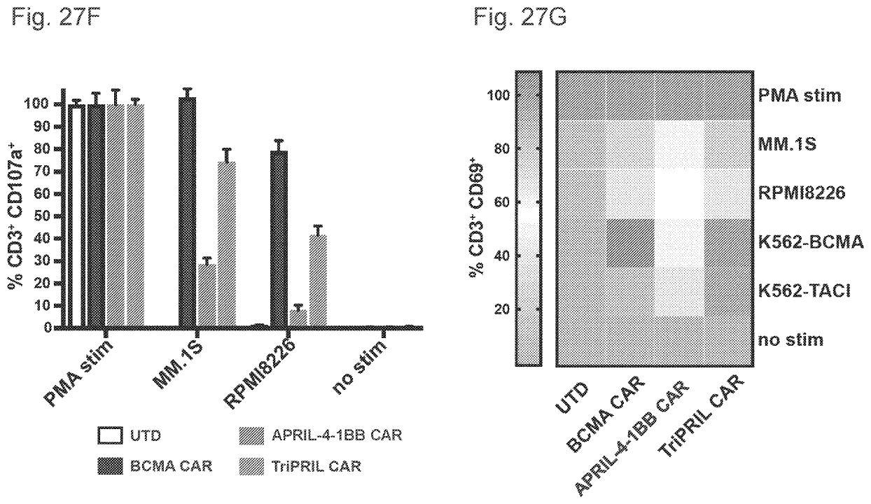

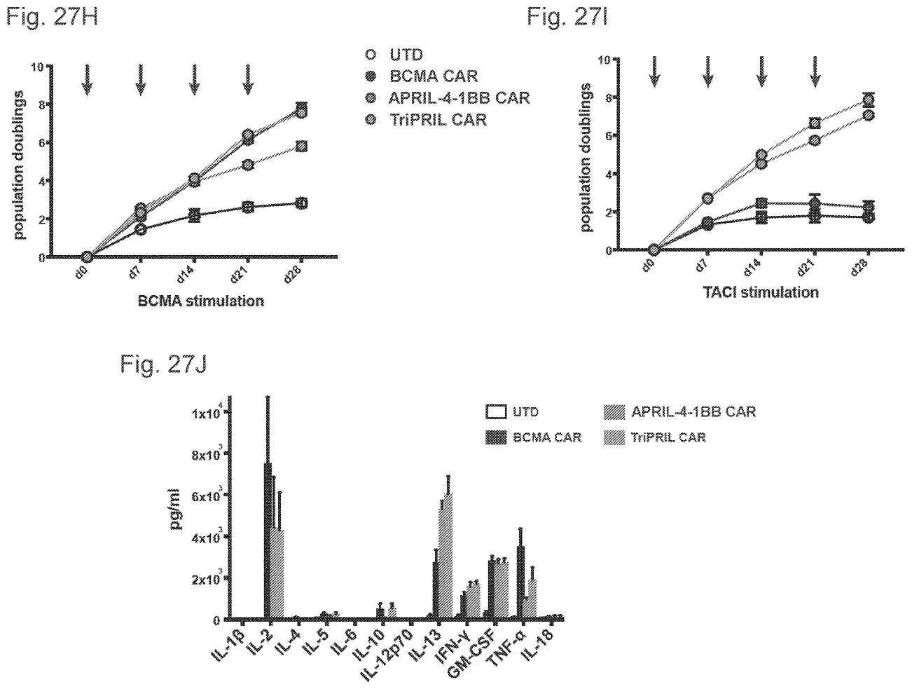

The BCMA, APRIL-4-1 BB, and TriPRIL CARs were tested for their binding and functional properties when transduced into human T cells. First, the binding affinities for soluble BCMA and soluble TACI was determined, as shown in FIG. 27A. The CAR T cells were incubated with soluble BCMA and soluble TACI conjugated to APC, and the binding affinity was determined based on mean fluorescence intensity (MFI). Binding affinity to sBCMA and sTACI was higher for TriPRIL CAR compared to APRIL-4-1 BB CAR. It was observed that BCMA CAR bound strongly to sBCMA and poorly to sTACI, consistent with the described binding characteristics of the antibody from which the scFv was derived. The APRIL CAR showed low binding affinity to both BCMA and TACI, whereas the TriPRIL CAR bound to both antigens, albeit with lower affinity to BCMA than the anti-BCMA CAR (FIG. 27A). Next, the three different CAR T cell constructs were tested for their cytotoxicity against the BCMA and/or TACI-expressing cell lines MM.1S (FIG. 27B), RPMI8226 (FIG. 27C), K562-BCMA (FIG. 27D), and K562-TACI (FIG. 27E). MM.1s (FIG. 27B) and RPMI8226 (FIG. 27C) MM cell lines were lysed at high efficiency by BCMA and TriPRIL CAR T cells, while specific lysis mediated by APRIL-4-1 BB CAR T cells was lower. For target cells expressing BCMA only, a similar pattern was observed: efficient lysis by the BCMA CARTs and the TriPRIL CAR T cells, with weaker lysis by the APRIL CARTs (FIG. 27D). In contrast, only the APRIL-4-1 BB and TriPRIL CAR T cells were able to lyse TACI only expressing target cells, though APRIL-mediated lysis still appeared weaker than TriPRIL-mediated lysis (FIG. 27E).

Degranulation (FIG. 27F) and activation (FIG. 27G) of the different CAR T cells were also determined. Cytotoxicity was measured in a luciferase-based killing assay, and degranulation and activation were analyzed by flow. Robust degranulation of the BCMA CAR T cells and the TriPRIL CAR T cells was observed when co-cultured with MM target cells, while the APRIL-4-1 BB CAR T cells degranulated only weakly, and untransduced T cells from the same donor in each experiment served as a negative control (FIG. 27F). Similarly, CD69 expression was induced in BCMA CAR T cells by co-culture with BCMA-positive target cells, while cells only positive for TACI did not cause their activation. APRIL-4-1 BB CAR T cells upregulated CD69 most in response to targets expressing high levels of TACI (K562-TACI), but only weakly in response to MM cell lines or BCMA. TriPRIL CAR T cells activated robustly in response target cells expressing either BCMA, TACI, or both (FIG. 27G). Not surprisingly, the degree of both CD107a and CD69 expression as a measure of T cell activation correlated with the expression of antigen (K562-transduced cells>MM.1s>RPMI8226), consistent with published data for CARs targeting CD22 (Haso et al., Blood. 121(7):1165-74 (2013)) and other antigens (Walker et al., Mol. Ther. 25(9):2189-2201 (2017); Arcangeli et al., Mol. Ther. 25(8):1933-1945 (2017); Caruso et al., Cancer Res. 75(17):3505-18 (2015)).

### Example 15. Long-Term Proliferation of CAR T Cells

Although short-term activation, lysis, and binding assays are bedrocks of CAR T cell characterization, the ability of CAR T cells to survive repeated antigen stimulation over time (Milone et al., Mol. Ther. 17(8):1453-64 (2009); Kalos et al., Sci. Transl. Med. 3(95):95ra73 (2009); Porter et al., Sci. Transl. Med. 7(303):303ra139 (2015)) and to produce polyfunctional cytokine responses (Rossi et al. Blood. 132(8):804-814 (2018)) are more discriminating assays of T cell function, and are thought to be more indicative of the types of functions required for efficacy in patients. Such CAR T cell persistence, proliferation, and polyfunctionality have also been correlated with clinical outcomes (Fraietta et al., Nat. Med. 24(5):563-571 (2018); Maude et al., N. Engl. J. Med. 371(16):1507-17 (2014); Neelapu et al., N. Engl. J. Med. 377(26):2531-2544 (2017); Rossi et al. Blood. 132(8):804-814 (2018)).

Long-term proliferation assays were performed to test the proliferative capacity of the different CAR T cells in response to repeated antigen stimulation with either BCMA or TACI, as shown in FIGS. 27H and 271. Starting from thawed CAR T cell preparation, weekly stimulation with K562-BCMA resulted in logarithmic growth of the BCMA and TriPRIL CAR T cells over 4 weeks; APRIL-4-1 BB CAR T cells grew logarithmically after 2 stimulations, but then tapered. In contrast, repeated K562-TACI stimulation only induced logarithmic growth of APRIL-4-1 BB and TriPRIL CAR T cells, with no significant difference between them (FIG. 27I). BCMA CAR T cells did not expand more than the untransduced control cells (p=0.5464) (FIG. 27I). Thus, one of the main drivers of the difference between APRIL-4-1 BB and TriPRIL CAR T cell function seems to be responsiveness to BCMA stimulation, whereas the main driver of differential function between anti-BCMA and TriPRIL function is responsiveness to TACI. The TriPRIL CAR outperformed APRIL CAR in cytotoxic potential, degranulation, activation, and long-term proliferation.

Lastly, cytokine production by the different CAR T cells upon co-culture with human MM.1S myeloma cells was measured in the supernatant by 12-plex Luminex assay (FIG. 27J). All three CAR constructs demonstrated robust antigen-specific production of Th1-type cytokines, like IL-2, IFNγ, GM-CSF and TNF-α, similar to other CAR T cell designs bearing 4-1 BB costimulation (Milone et al., Mol. Ther. 17(8):1453-64 (2009); Scarfo et al., Blood. 132(14):1495-1506 (2018)).

### Example 16. In Vivo Activity of CAR T Cells

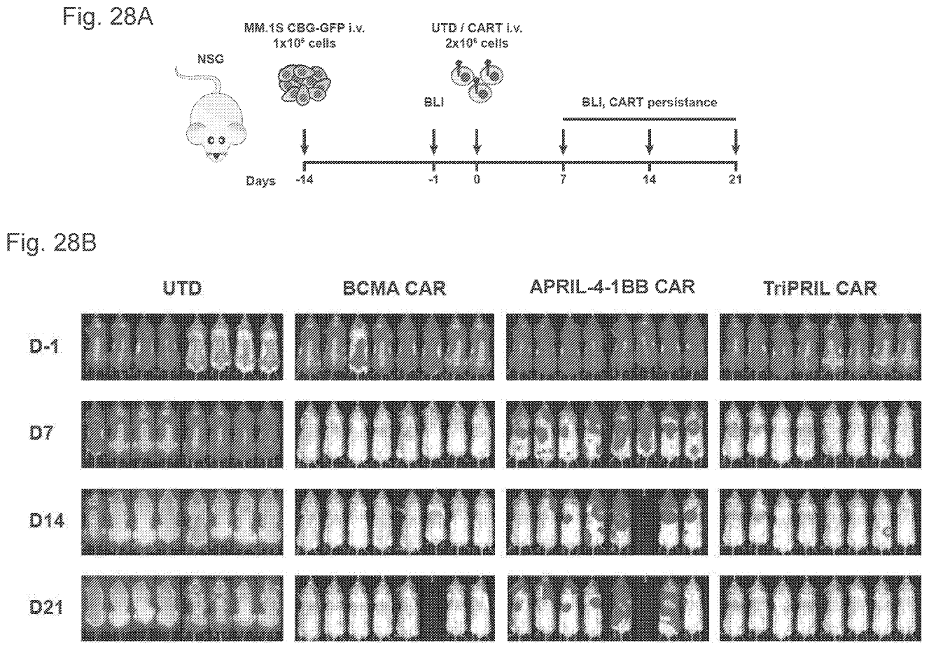

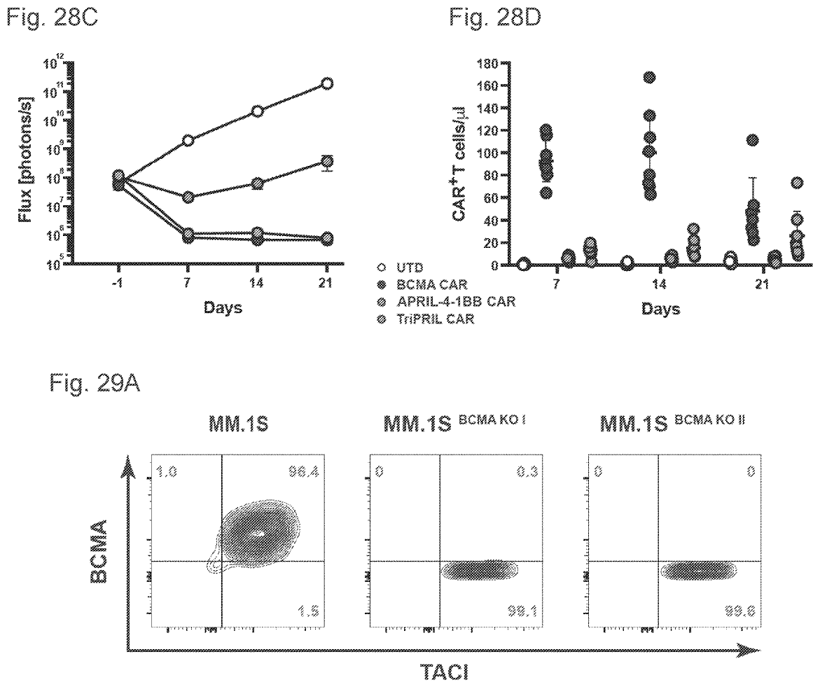

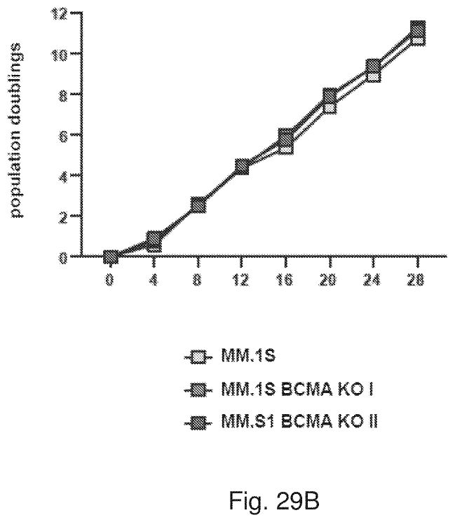

The anti-tumor efficiency of the CAR T cells was assessed in a xenograft model of multiple myeloma by engrafting NSG mice with high tumor burden of MM.1S myeloma cells. NSG mice were injected with 1×10⁶ MM.1 S myeloma cells, modified to express click beetle green luciferase (CBG-luc), and tumors engrafted over 14 days. Tumor burden was monitored by bioluminescence imaging (BLI) over time. After tumor engraftment and randomization, the mice were injected on day 0 with a single dose intravenous (i.v.) dose of 2×10⁶ of either untransduced (UTD), BCMA CAR, APRIL-4-1 BB CAR, or TriPRIL CAR T cells from the same donor (FIG. 28A). Tumor burden was monitored weekly by BLI, and CAR T cell persistence was measured in peripheral blood weekly by flow cytometry. While tumor burden continuously progressed in the UTD treated group, all CART treated mice showed anti-tumor responses. FIGS. 28B and 28C show the representative bioluminescence imaging and quantification of flux over time. In this high tumor-burden model, BCMA and TriPRIL CARTs were able to eradicate the tumors, while APRIL-4-1 BB CAR T cells only led to a stabilization of tumor burden (FIG. 28B). Treatment response in all 3 groups receiving CAR T cells was statistically significant in relation to the UTD control group. There was no significant difference between BCMA and TriPRIL CAR T cell treated animals (p=0.8451) in terms of tumor response (FIG. 28C). The persistence of the CAR T cells was measured in the peripheral blood by flow cytometry (FIG. 28D). In the peripheral blood, BCMA CAR T cell numbers showed a rapid increase and then contraction at day 14 following CART administration. In contrast, the TriPRIL CAR T cells underwent slower expansion kinetics with cell numbers still increasing on day 21 post CAR T cell administration. UTD and APRIL-4-1 BB CAR T cells did not show measurable expansion in the blood at the analyzed time points (FIG. 28D). Together these data indicated that our APRIL-based CARs were not likely to be optimally functional against MM cells bearing BCMA or TACI, and further studies were focused on comparing TriPRIL CAR T cells relative to BCMA, and in particular their responsiveness to MM with loss of BCMA. The TriPRIL CAR and BCMA CAR T cells were able to eradicate the tumors while the APRIL-4-1 BB CAR T cells only led to a stabilization of tumor burden.

### Example 17. Modeling BCMA Antigen Escape

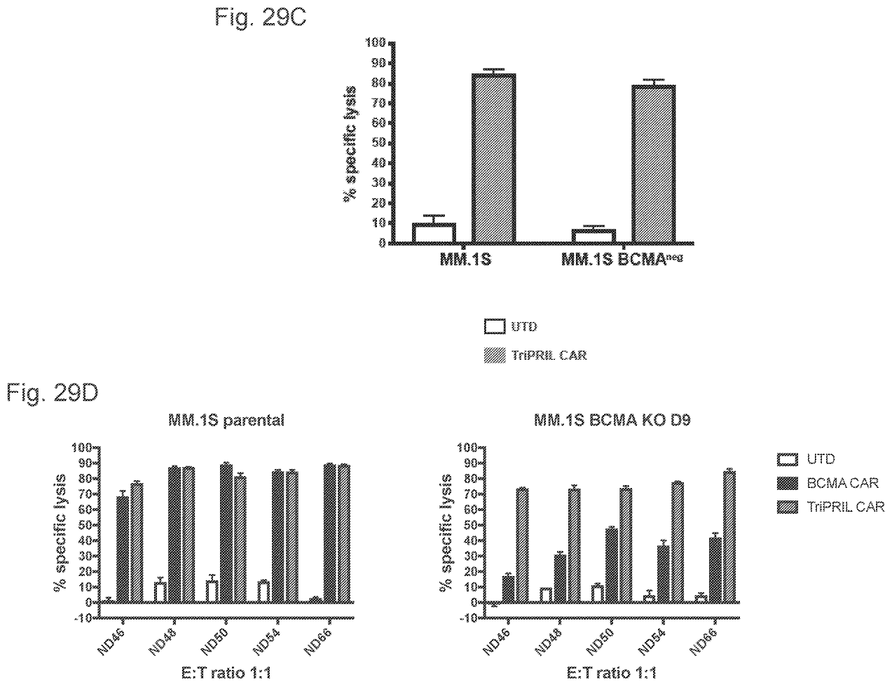

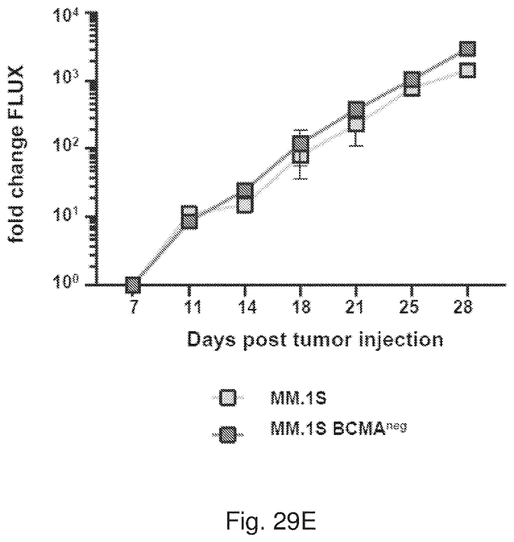

To model BCMA antigen escape, MM.1 S myeloma cells with a CRISPR/Cas9-mediated BCMA knockout (KO) were generated. As shown in FIG. 29A, lack of BCMA expression was confirmed, while TACI expression levels remained unaffected in the knockout cell line. The population doubling of MM.1 S, MM.1S BCMA KO I, and MM.1S BCMA KO II cell lines is shown in FIG. 29B. Parental and BCMA KO MM.1 S myeloma cells showed identical growth kinetics in vitro, independently of the guide RNA used. In addition, tumorigenicity and growth kinetics of MM.1S cells and MM.1S BCMA KO cells were essentially identical in NSG mice (FIG. 29E).

Next, the killing of MM.1S and MM.1 S BCMA knockout cells by BCMA and TriPRIL CAR T cells was tested in vitro. TriPRIL CAR effectively lysed MM.1S and MM.1S BCMA KO cells compared to UTD cells (FIG. 29C). As demonstrated in FIG. 29D, killing of MM.1S BCMA knockout cells by BCMA CAR T cells is significantly reduced as compared to killing of MM.1S cells, while killing by TriPRIL CAR T cells was undiminished between the two cell lines (p=0.8986). The kinetics of tumor growth over time of MM.1 S and MM.1S BCMA-negative cell lines in NSG mice is shown in FIG. 29E, and demonstrates no change.

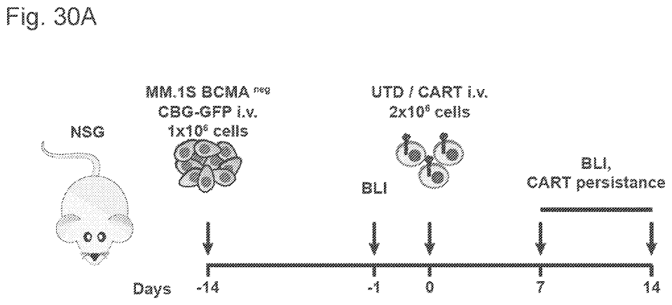

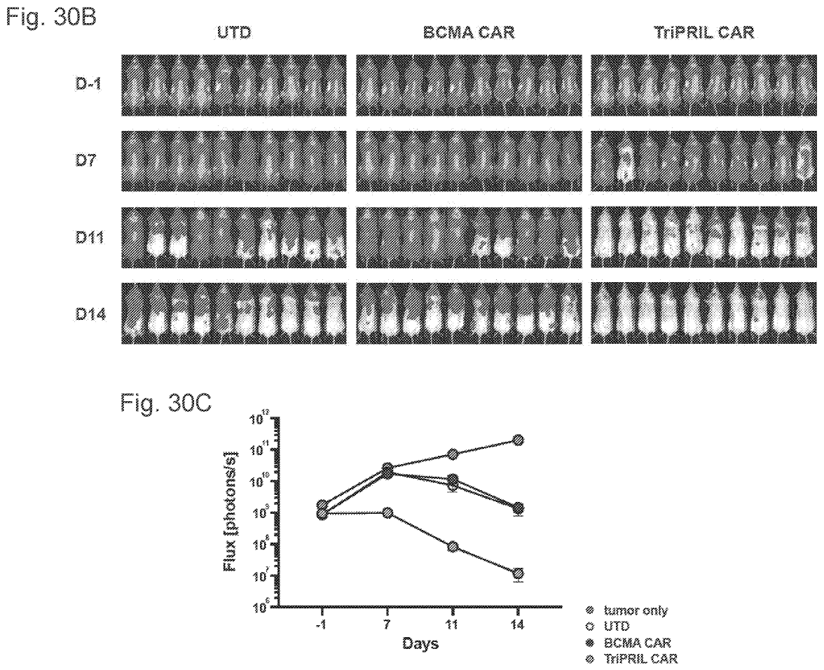

The BCMA antigen escape model was also tested in vivo, for which the experimental design is shown in FIG. 30A. NSG mice were injected with 1×10⁶ MM.1S BCMA knockout cells and allowed to engraft for 14 days. Tumor burden was monitored over time. Following confirmation of tumor engraftment by BLI, mice were injected on day 0 with a single i.v. dose of 2×10⁶ untransduced, BCMA CAR, or TriPRIL CAR cells. (FIG. 30). In addition, a group of mice was left untreated to assess tumorigenicity and control for allogeneic rejection, which occurs frequently with MM models, and limits the evaluable duration of in vivo experiments. The tumor burden was monitored by BLI over time. Over the course of the experiment, all treated mice showed a tumor regression, while disease burden in the “tumor only” group continuously progressed, indicating a non-antigen-specific allogeneic reaction against the tumor through the endogenous T cell receptor. Representative bioluminescence imaging is shown in FIG. 30B, and quantification of flux in the three groups is shown in FIG. 30C. Again, it was observed that TriPRIL CAR T cells was efficacious against MM.1S BCMA knockout cells compared to untransduced and BCMA CAR T cells. Only the TriPRIL CAR T treated mice cleared the tumors by day 14 (FIG. 30B), consistent with antigen-specific mediated responses induced by CAR T cells. Quantification of tumor burden on day 7 showed a statistically a significant difference between groups receiving TriPRIL CAR T cells and those receiving BCMA CAR T cells or UTD control cells (p<0.0001) (FIG. 30C). For reasons that remain unclear, allogeneic responses of T cells against MM.1S BCMA-knockout cells were observed earlier compared to models that used parental MM.1S cells.

The APRIL-based CARs were able to redirect T cell cytotoxicity to both BCMA and TACI positive tumor cells. Since both these receptors are consistently upregulated on malignant plasma cells, this is an attractive method to target MM. Furthermore, it was observed that using a trimeric form of APRIL rather than monomeric form as the CAR binding domain increased recognition of MM antigens in vitro and in vivo.

### Example 18. Polyfunctionality of CAR T Cells

Polyfunctional cytokine production at the single cell level has more recently emerged as a correlative function between functional CAR T cell products that induce clinical responses in patients with lymphoma (Rossi et al. Blood. 132(8):804-814 (2018)) and can be more indicative of the types of functions required for efficacy in patients. CAR T cell polyfunctionality have also been correlated with clinical outcomes. It is likely that polyfunctionality is a function of both starting T cell product and CAR design.

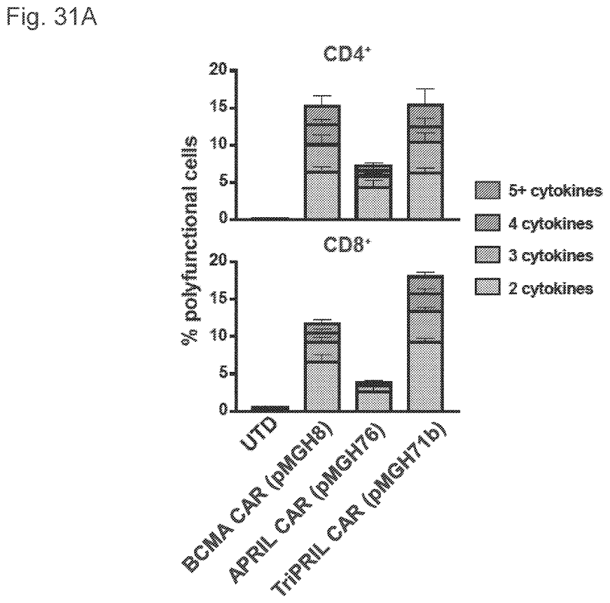

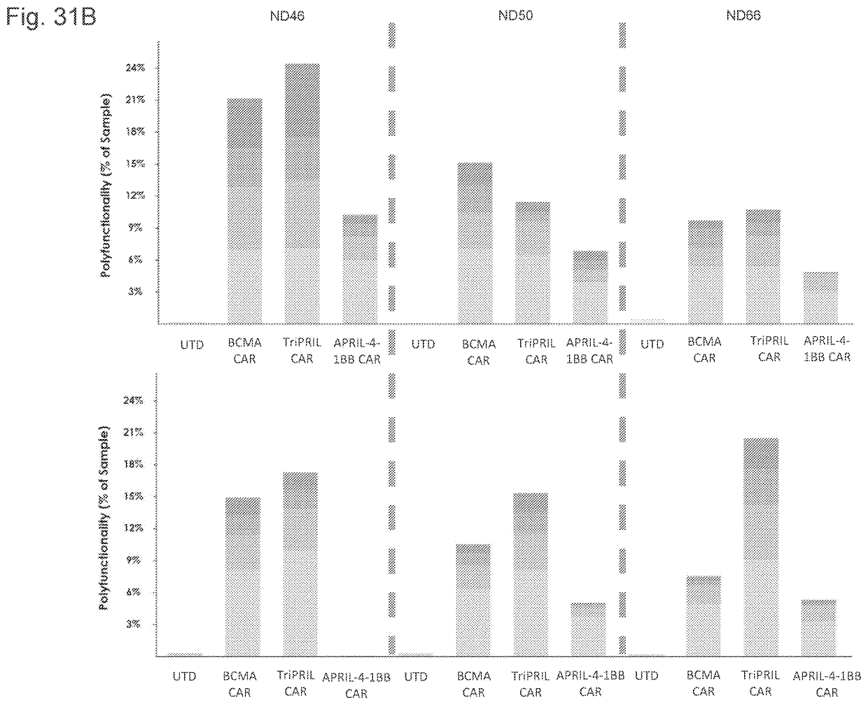

The percentage of polyfunctional CD4+ and CD8+ T cells was measured in each CAR T cell product upon BCMA and TACI stimulation with a 32-plex antibody assay. Polyfunctionality was defined as secretion of ≥2 cytokines. Polyfunctional upregulation was observed in both CD4+ and CD8+ CAR T cells with BCMA CAR, APRIL-4-1 BB CAR, and TriPRIL CAR constructs across donors compared to untransduced cells upon BCMA stimulation (FIGS. 31A and 31B). The CD4+ subset of BCMA CAR T cells and TriPRIL CAR T cells both were made up of 10-24% of polyfunctional cells, while the percentage polyfunctional APRIL CARTs was significantly lower (5-10%; p=0.0064). Differences in the CD8+ subset were more pronounced, with the TriPRIL CAR T cell product containing significantly more polyfunctional cells (16-21%) than the BCMA CAR T cells (8-15%; p=0.0003) and the APRIL CAR T cells (0-6%; p<0.0001) product (FIG. 31A). APRIL-4-1 BB CAR showed the lowest polyfunctional profile among the three CAR constructs, whereas TriPRIL in CD8+ CAR T cell products had the highest polyfunctionality. TriPRIL CAR and BCMA CAR exhibited comparable levels in CD4+ CAR T cell products by BCMA antigen stimulation.

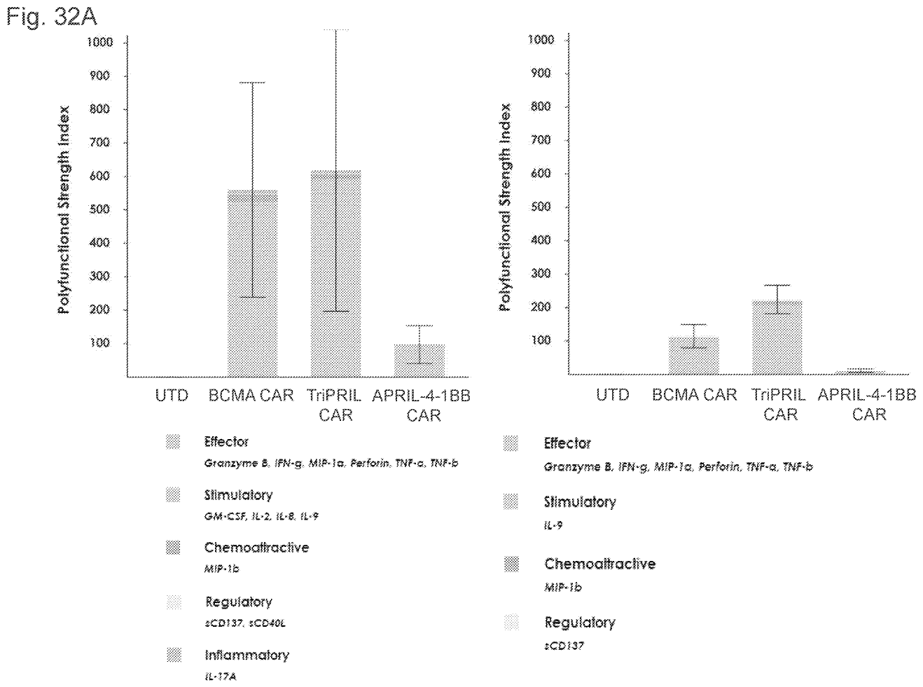

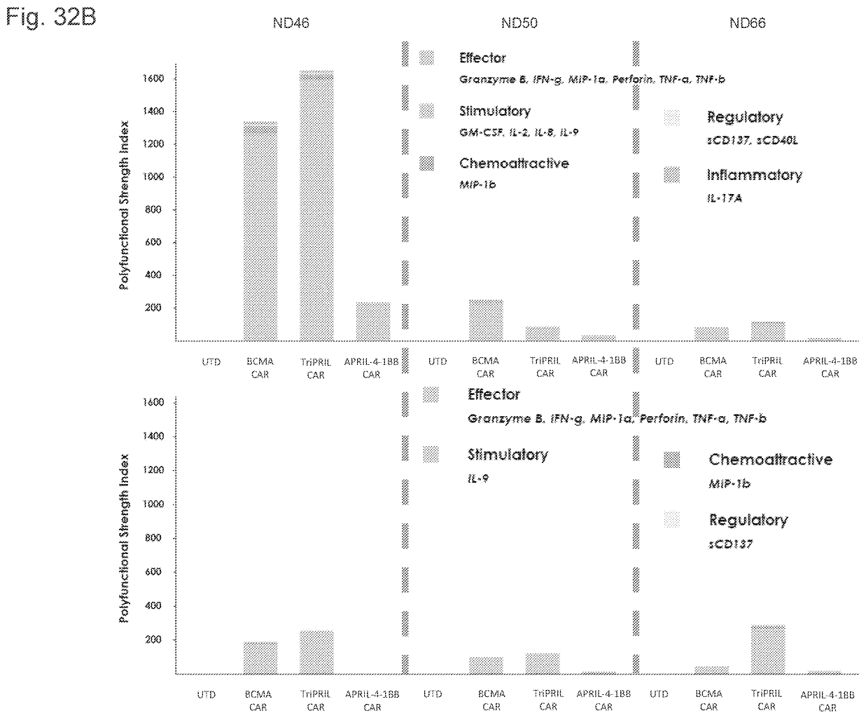

In line with the polyfunctional upregulation, BCMA-specific upregulation of polyfunctional strength index (PSI) was observed in both CD4+ and CD8+ CAR T cells with BCMA CAR, APRIL-4-1 BB CAR, and TriPRIL CAR constructs across donors compared to untransduced cells upon BCMA antigen stimulation (FIGS. 32A and 32B). TriPRIL CAR had the highest PSI, while APRIL-4-1 BB CAR showed the least PSI increase among the three constructs in both CD4+ and CD8+ CAR T cells upon BCMA antigen stimulation. The enhanced PSI of both CD4+ and CD8+ CAR T cells was predominated by effector proteins including Granzyme B, IFN-γ, MIP-1a, perforin, TNF-α, and TNF-β. The low levels of GM-CSF, IL-2, IL-8, sCD40L, and IL-17A secretions were uniquely composed in CD4 PSI, while IL-9, MIP-1 b, and sCD137 were identified in both CD4 and CD8 PSI.

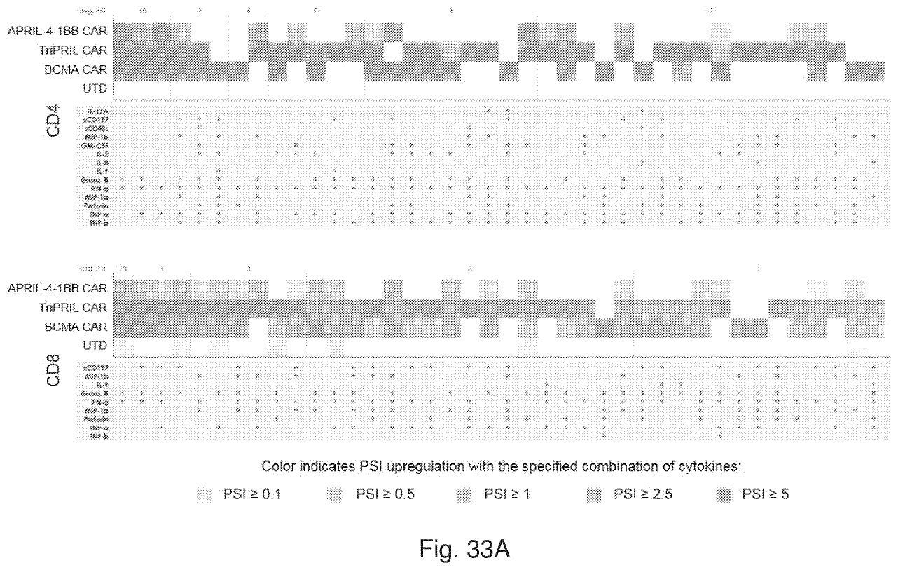

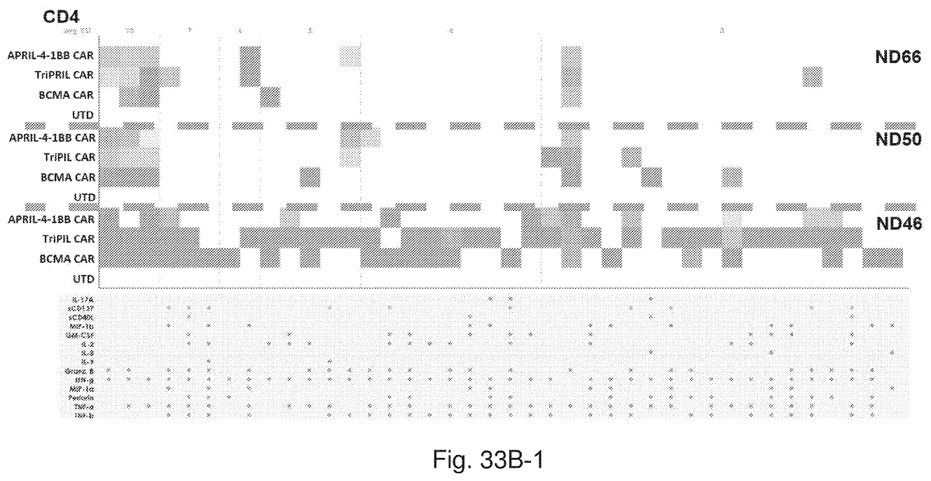

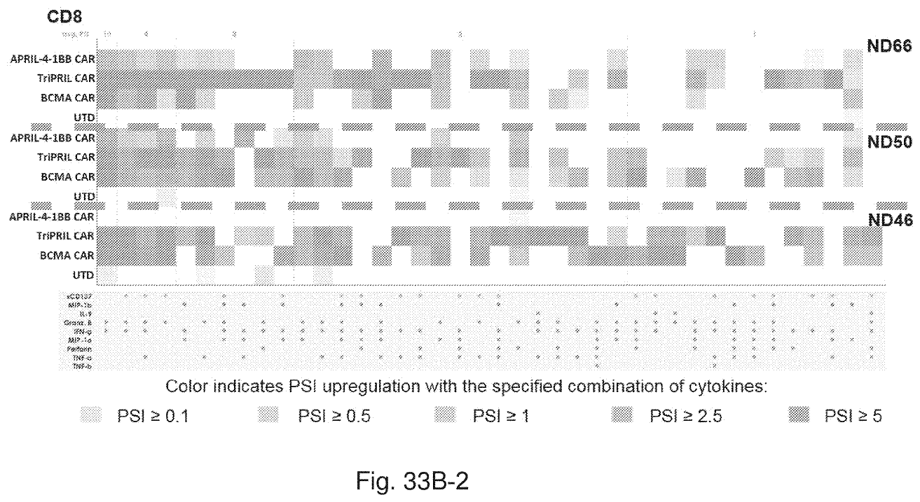

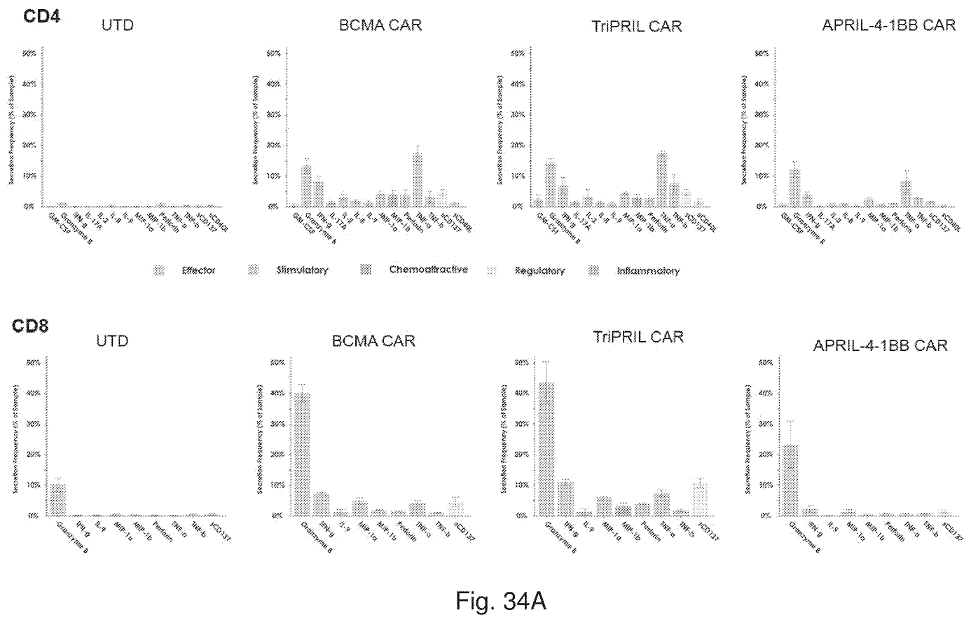

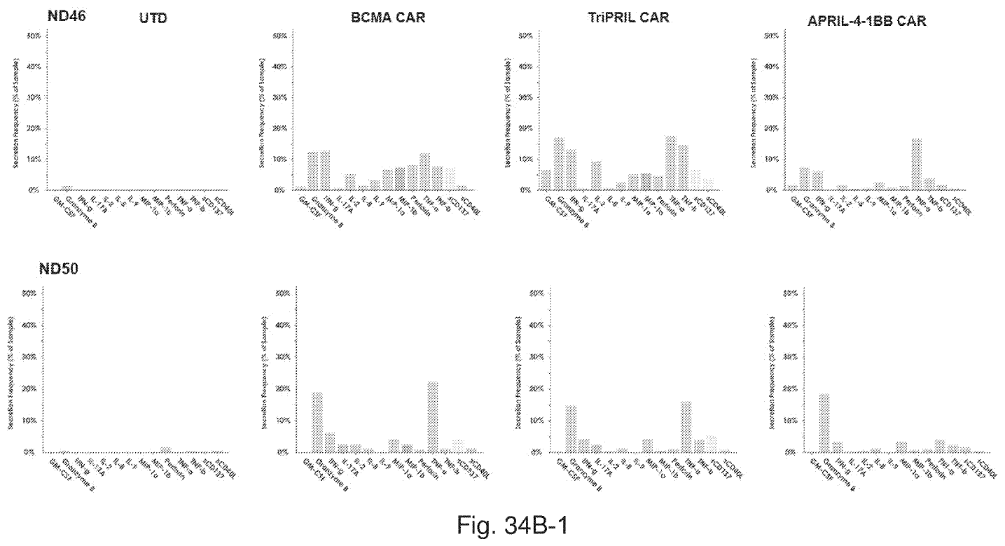

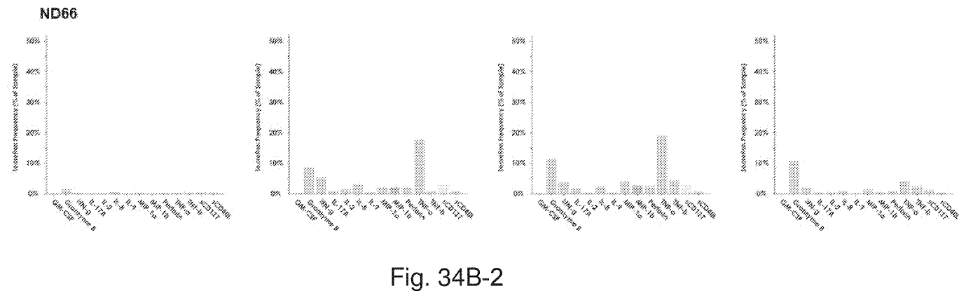

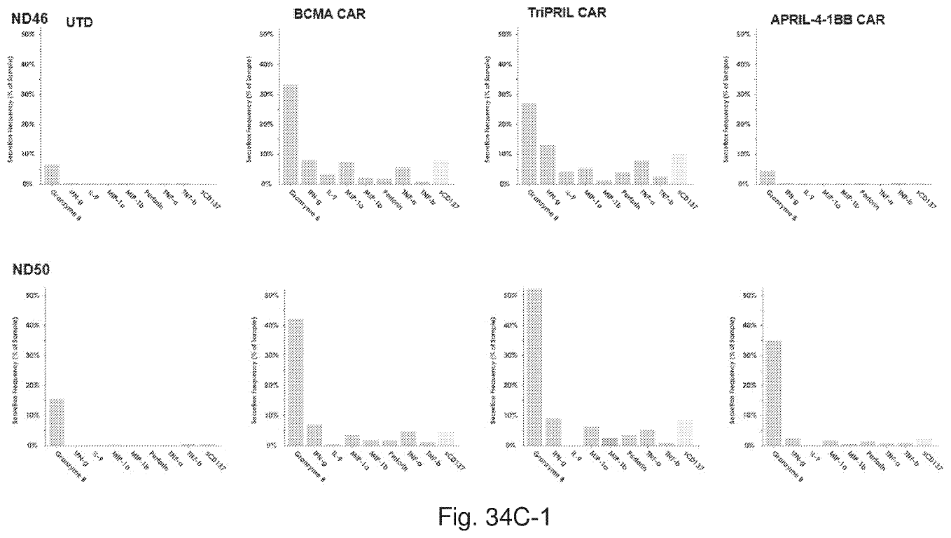

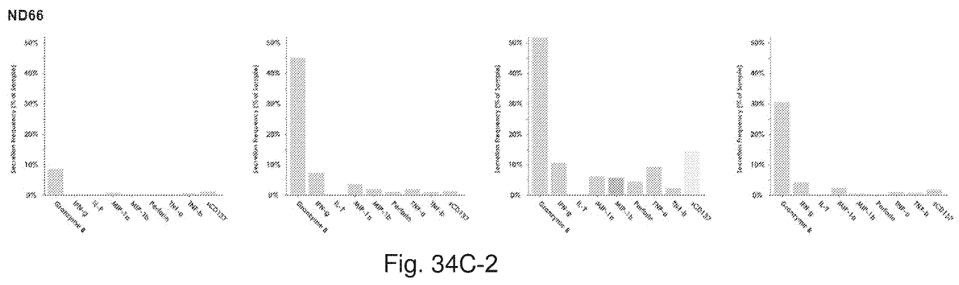

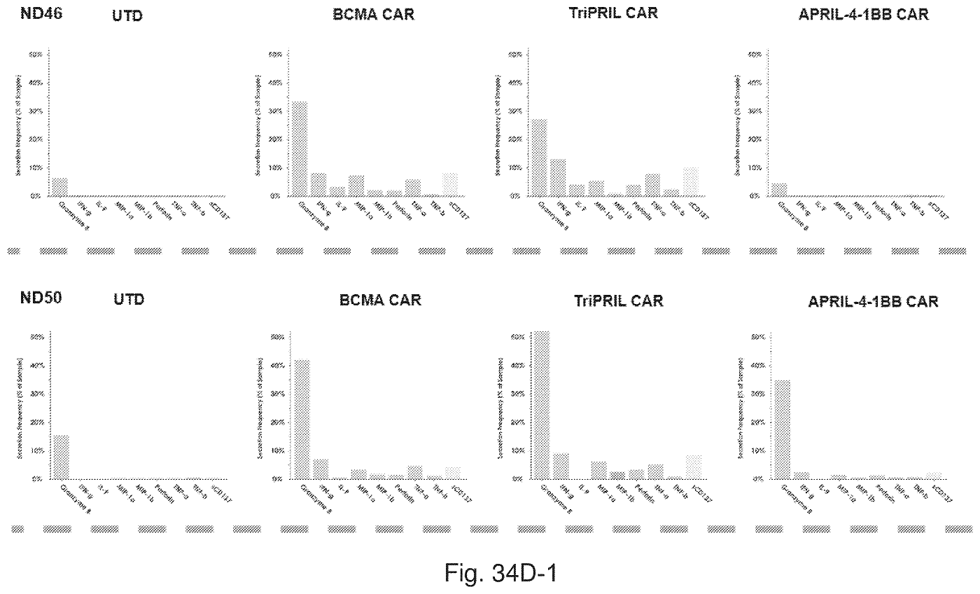

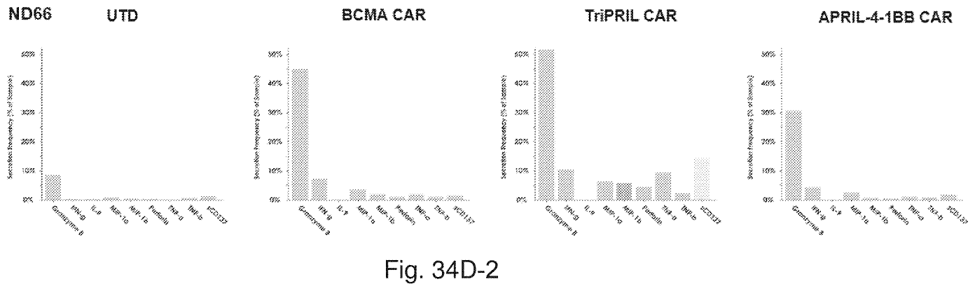

Polyfunctional heatmaps (FIGS. 33A and 33B) further reveal that the enhanced polyfunctional cell subsets have distinct protein combinations in both CD4+ and CD8+ CAR T cells. Further, an increase of a variety of protein secretions was observed in both CD4+ and CD8+ CAR T cells with various CAR constructs (FIGS. 34A-34D). Single cell analysis of individual cytokine profiles demonstrated that the predominant cytokines and measured proteins produced were classified as effector cytokines in both CD4+ and CD8+ T cells, with similar profiles but lower frequencies among the CAR constructs tested (FIG. 34A).

### Example 19. TriPRIL CAR T Cell Functions in Hostile MM Environments

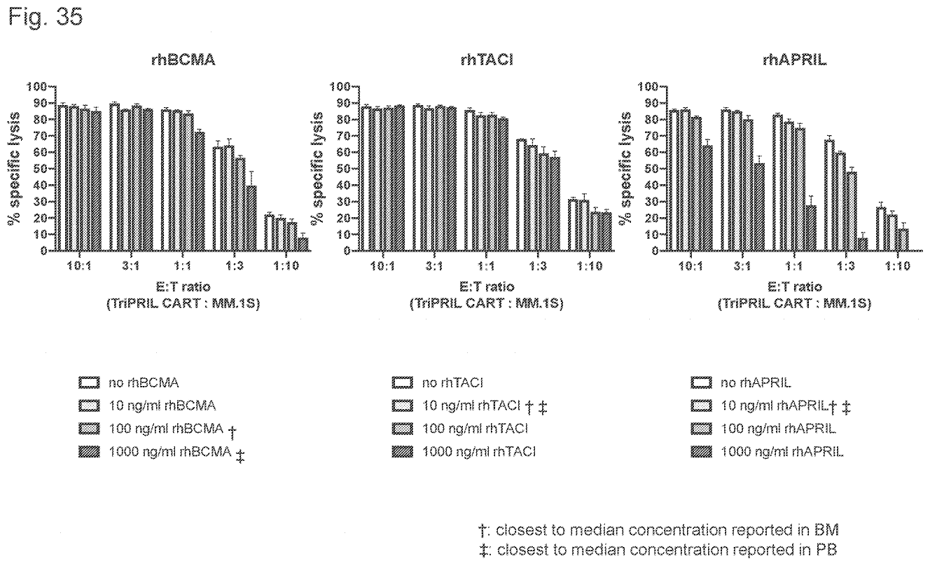

In the bone marrow niche, APRIL is secreted from bone marrow stromal cells and provides growth signals for plasma cells. Supraphysiologic concentrations of sBCMA, sTACI and sAPRIL have been reported in the bone marrow and peripheral blood of MM patients. It was determined whether sBCMA, sTACI and sAPRIL blocks TriPRIL CAR T cell lysis against MM cells that express membrane-bound BCMA and TACI. To this end, TriPRIL CAR T cells were co-cultured with MM.1S target cells at different effector to target ratios over a range of concentrations of sBCMA, sTACI and sAPRIL in the culture. At the highest concentrations (1000 ng/ml) of sBCMA and sAPRIL, reduced cytotoxicity of TriPRIL CAR was observed only at the lowest effector:target ratios tested (1:3 and 1:10) (FIG. 35). Importantly, the concentrations of sTACI and sAPRIL that we tested far exceed the levels reported in MM patients. However, the highest concentration of sBCMA that was tested is comparable to sBCMA concentrations reported in some patients with advanced MM. These data indicate that binding of sBCMA to TriPRIL may inhibit its activity at low E:T ratios, but this may potentially be overcome with higher doses of TriPRIL CAR T cells or reducing the concentration of sBCMA in the patients with other therapies, such as chemotherapy that is also useful as conditioning lymphodepletion.

### Example 20. TriPRIL CAR Nucleic Acid Sequences

pMGH71b-TriPRIL CAR (SEQ ID NO: 45):
EF1α promoter (SEQ ID NO: 46 (nucleotides 1-1184 of SEQ ID NO: 45));
CD8 signal peptide (SEQ ID NO: 47 (nucleotides 1185-1247 of SEQ ID NO: 45));
TriPRIL (truncated APRIL (SEQ ID NO: 48 (nucleotides 1248-1655 of SEQ ID NO: 45))-
Gly/Ser linker (SEQ ID NO: 49 (nucleotides 1656-1691 of SEQ ID NO: 45))-
truncated APRIL (SEQ ID NO: 50 (nucleotides 1692-2099 of SEQ ID NO: 45))-
Gly/Ser linker (SEQ ID NO: 51 (nucleotides 2100-2135 of SEQ ID NO: 45))-
truncated APRIL (SEQ ID NO: 52 (nucleotides 2136-2543 of SEQ ID NO: 45)));
CD8 hinge + transmembrane domains (SEQ ID NO: 53 (nucleotides 2544-2750 of SEQ ID NO: 45));
4-1BB co-stimulatory domain (SEQ ID NO: 54 (nucleotides 2751-2876 of SEQ ID NO: 45));
CD3ζ signaling domain (SEQ ID NO: 55 (nucleotides 2877-3212 of SEQ ID NO: 45))
(SEQ ID NO: 45)
CGTGAGGCTCCGGTGCCCGTCAGTGGGCAGAGCGCACATCGCCCACAGTCCCCGAGAAGTTG
GGGGGAGGGGTCGGCAATTGAACCGGTGCCTAGAGAAGGTGGCGCGGGGTAAACTGGGAAAG
TGATGTCGTGTACTGGCTCCGCCTTTTTCCCGAGGGTGGGGGAGAACCGTATATAAGTGCAGTA
GTCGCCGTGAACGTTCTTTTTCGCAACGGGTTTGCCGCCAGAACACAGGTAAGTGCCGTGTGTG
GTTCCCGCGGGCCTGGCCTCTTTACGGGTTATGGCCCTTGCGTGCCTTGAATTACTTCCACCTG
GCTGCAGTACGTGATTCTTGATCCCGAGCTTCGGGTTGGAAGTGGGTGGGAGAGTTCGAGGCC
TTGCGCTTAAGGAGCCCCTTCGCCTCGTGCTTGAGTTGAGGCCTGGCCTGGGCGCTGGGGCCG
CCGCGTGCGAATCTGGTGGCACCTTCGCGCCTGTCTCGCTGCTTTCGATAAGTCTCTAGCCATT
TAAAATTTTTGATGACCTGCTGCGACGCTTTTTTTCTGGCAAGATAGTCTTGTAAATGCGGGCCA
AGATCTGCACACTGGTATTTCGGTTTTTGGGGCCGCGGGCGGCGACGGGGCCCGTGCGTCCCA
GCGCACATGTTCGGCGAGGCGGGGCCTGCGAGCGCGGCCACCGAGAATCGGACGGGGGTAG
TCTCAAGCTGGCCGGCCTGCTCTGGTGCCTGGCCTCGCGCCGCCGTGTATCGCCCCGCCCTGG
GCGGCAAGGCTGGCCCGGTCGGCACCAGTTGCGTGAGCGGAAAGATGGCCGCTTCCCGGCCC
TGCTGCAGGGAGCTCAAAATGGAGGACGCGGCGCTCGGGAGAGCGGGCGGGTGAGTCACCCA
CACAAAGGAAAAGGGCCTTTCCGTCCTCAGCCGTCGCTTCATGTGACTCCACTGAGTACCGGGC
GCCGTCCAGGCACCTCGATTAGTTCTCGTGCTTTTGGAGTACGTCGTCTTTAGGTTGGGGGGAG
GGGTTTTATGCGATGGAGTTTCCCCACACTGAGTGGGTGGAGACTGAAGTTAGGCCAGCTTGGC
ACTTGATGTAATTCTCCTTGGAATTTGCCCTTTTTGAGTTTGGATCTTGGTTCATTCTCAAGCCTC
AGACAGTGGTTCAAAGTTTTTTTCTTCCATTTCAGGTGTCGTGAATGGCCCTCCCTGTCACCGCC
CTGCTGCTTCCGCTGGCTCTTCTGCTCCACGCCGCTCGGCCCCACTCAGTACTCCATCTCGTTC
CTATCAATGCAACAAGCAAGGATGATTCTGACGTGACTGAGGTTATGTGGCAACCTGCCCTCCG
AAGGGGTAGAGGTCTCCAGGCTCAGGGGTACGGCGTCCGCATCCAGGATGCAGGAGTTTACTT
GCTCTATAGTCAGGTTCTCTTTCAGGATGTCACATTCACTATGGGGCAGGTTGTAAGCCGGGAA
GGCCAAGGTAGACAGGAGACTCTTTTTCGATGCATCAGGAGTATGCCTTCACATCCAGACCGAG
CGTACAATTCCTGCTACTCTGCTGGTGTTTTCCATCTGCACCAAGGTGACATTCTGTCCGTCATA
ATTCCGAGAGCTAGAGCCAAGCTTAACCTCAGCCCACACGGGACCTTTCTGGGCTTCGTTAAGC
TCGGCGGAGGCTCTGGGGGAGGGTCCGGGGGCGGGAGTCATTCAGTGCTTCACCTCGTCCCG
ATTAACGCAACAAGCAAAGATGACTCCGACGTGACTGAAGTGATGTGGCAGCCAGCATTGAGGC
GAGGTAGAGGTCTCCAGGCTCAAGGATATGGTGTCAGAATACAGGATGCAGGAGTTTATCTCCT
GTACAGTCAGGTGTTGTTTCAGGATGTTACTTTTACTATGGGCCAAGTTGTAAGTAGAGAAGGTC
AGGGAAGGCAAGAGACGCTCTTCAGGTGCATACGAAGTATGCCCAGTCACCCTGATAGAGCATA
CAACTCTTGCTACAGTGCGGGCGTTTTTCATTTGCACCAGGGAGATATCCTCAGCGTGATCATCC
CAAGAGCACGCGCAAAATTGAACCTCTCACCACACGGTACCTTTCTCGGTTTTGTCAAGCTTGGA
GGCGGATCAGGAGGGGGCAGCGGCGGGGGCTCTCACTCAGTTTTGCATCTCGTCCCTATCAAC
GCCACGAGCAAGGACGATTCAGATGTGACTGAAGTCATGTGGCAGCCGGCCCTTCGGAGGGGA
AGAGGGTTGCAAGCTCAGGGTTATGGGGTGCGAATACAGGACGCAGGGGTGTACCTCCTCTAT
TCTCAGGTATTGTTCCAAGACGTAACCTTCACGATGGGTCAAGTCGTCTCCCGAGAAGGTCAAG
GGCGCCAAGAAACCCTTTTTAGGTGCATTAGAAGCATGCCAAGCCATCCTGACCGCGCATATAA
CTCATGTTACTCCGCCGGCGTGTTTCACTTGCACCAAGGTGATATCCTTAGCGTTATTATTCCGC
GAGCGCGGGCCAAGCTGAATCTTTCACCGCACGGGACCTTCCTTGGGTTTGTAAAACTGACCAC
TACCCCAGCACCGAGGCCACCCACCCCGGCTCCTACCATCGCCTCCCAGCCTCTGTCCCTGCG
TCCGGAGGCATGTAGACCCGCAGCTGGTGGGGCCGTGCATACCCGGGGTCTTGACTTCGCCTG
CGATATCTACATTTGGGCCCCTCTGGCTGGTACTTGCGGGGTCCTGCTGCTTTCACTCGTGATC
ACTCTTTACTGTAAGCGCGGTCGGAAGAAGCTGCTGTACATCTTTAAGCAACCCTTCATGAGGC
CTGTGCAGACTACTCAAGAGGAGGACGGCTGTTCATGCCGGTTCCCAGAGGAGGAGGAAGGCG
GCTGCGAACTGCGCGTGAAATTCAGCCGCAGCGCAGATGCTCCAGCCTACCAACAGGGGCAGA
ACCAGCTCTACAACGAACTCAATCTTGGTCGGAGAGAGGAGTACGACGTGCTGGACAAGCGGA
GAGGACGGGACCCAGAAATGGGCGGGAAGCCGCGCAGAAAGAATCCCCAAGAGGGCCTGTAC
AACGAGCTCCAAAAGGATAAGATGGCAGAAGCCTATAGCGAGATTGGTATGAAAGGGGAACGCA
GAAGAGGCAAAGGCCACGACGGACTGTACCAGGGACTCAGCACCGCCACCAAGGACACCTATG
ACGCTCTTCACATGCAGGCCCTGCCGCCTCGG
EF1α promoter (nucleotides 1-1184 of SEQ ID NO: 45)
(SEQ ID NO: 46)
CGTGAGGCTCCGGTGCCCGTCAGTGGGCAGAGCGCACATCGCCCACAGTCCCCGAGAAGTTG
GGGGGAGGGGTCGGCAATTGAACCGGTGCCTAGAGAAGGTGGCGCGGGGTAAACTGGGAAAG
TGATGTCGTGTACTGGCTCCGCCTTTTTCCCGAGGGTGGGGGAGAACCGTATATAAGTGCAGTA
GTCGCCGTGAACGTTCTTTTTCGCAACGGGTTTGCCGCCAGAACACAGGTAAGTGCCGTGTGTG
GTTCCCGCGGGCCTGGCCTCTTTACGGGTTATGGCCCTTGCGTGCCTTGAATTACTTCCACCTG
GCTGCAGTACGTGATTCTTGATCCCGAGCTTCGGGTTGGAAGTGGGTGGGAGAGTTCGAGGCC
TTGCGCTTAAGGAGCCCCTTCGCCTCGTGCTTGAGTTGAGGCCTGGCCTGGGCGCTGGGGCCG
CCGCGTGCGAATCTGGTGGCACCTTCGCGCCTGTCTCGCTGCTTTCGATAAGTCTCTAGCCATT
TAAAATTTTTGATGACCTGCTGCGACGCTTTTTTTCTGGCAAGATAGTCTTGTAAATGCGGGCCA
AGATCTGCACACTGGTATTTCGGTTTTTGGGGCCGCGGGCGGCGACGGGGCCCGTGCGTCCCA
GCGCACATGTTCGGCGAGGCGGGGCCTGCGAGCGCGGCCACCGAGAATCGGACGGGGGTAG
TCTCAAGCTGGCCGGCCTGCTCTGGTGCCTGGCCTCGCGCCGCCGTGTATCGCCCCGCCCTGG
GCGGCAAGGCTGGCCCGGTCGGCACCAGTTGCGTGAGCGGAAAGATGGCCGCTTCCCGGCCC
TGCTGCAGGGAGCTCAAAATGGAGGACGCGGCGCTCGGGAGAGCGGGCGGGTGAGTCACCCA
CACAAAGGAAAAGGGCCTTTCCGTCCTCAGCCGTCGCTTCATGTGACTCCACTGAGTACCGGGC
GCCGTCCAGGCACCTCGATTAGTTCTCGTGCTTTTGGAGTACGTCGTCTTTAGGTTGGGGGGAG
GGGTTTTATGCGATGGAGTTTCCCCACACTGAGTGGGTGGAGACTGAAGTTAGGCCAGCTTGGC
ACTTGATGTAATTCTCCTTGGAATTTGCCCTTTTTGAGTTTGGATCTTGGTTCATTCTCAAGCCTC
AGACAGTGGTTCAAAGTTTTTTTCTTCCATTTCAGGTGTCGTGA
CD8 signal peptide (nucleotides 1185-1247 of SEQ ID NO: 45)
(SEQ ID NO: 47)
ATGGCCCTCCCTGTCACCGCCCTGCTGCTTCCGCTGGCTCTTCTGCTCCACGCCGCTCGGCCC
Truncated APRIL (nucleotides 1248-1655 of SEQ ID NO: 45)
(SEQ ID NO: 48)
CACTCAGTACTCCATCTCGTTCCTATCAATGCAACAAGCAAGGATGATTCTGACGTGACTGAGGT
TATGTGGCAACCTGCCCTCCGAAGGGGTAGAGGTCTCCAGGCTCAGGGGTACGGCGTCCGCAT
CCAGGATGCAGGAGTTTACTTGCTCTATAGTCAGGTTCTCTTTCAGGATGTCACATTCACTATGG
GGCAGGTTGTAAGCCGGGAAGGCCAAGGTAGACAGGAGACTCTTTTTCGATGCATCAGGAGTAT
GCCTTCACATCCAGACCGAGCGTACAATTCCTGCTACTCTGCTGGTGTTTTCCATCTGCACCAAG
GTGACATTCTGTCCGTCATAATTCCGAGAGCTAGAGCCAAGCTTAACCTCAGCCCACACGGGAC
CTTTCTGGGCTTCGTTAAGCTC
Gly/Ser linker (nucleotides 1656-1691 of SEQ ID NO: 45)
(SEQ ID NO: 49)
GGCGGAGGCTCTGGGGGAGGGTCCGGGGGCGGGAGT
Truncated APRIL (nucleotides 1692-2099 of SEQ ID NO: 45)
(SEQ ID NO: 50)
CATTCAGTGCTTCACCTCGTCCCGATTAACGCAACAAGCAAAGATGACTCCGACGTGACTGAAG
TGATGTGGCAGCCAGCATTGAGGCGAGGTAGAGGTCTCCAGGCTCAAGGATATGGTGTCAGAA
TACAGGATGCAGGAGTTTATCTCCTGTACAGTCAGGTGTTGTTTCAGGATGTTACTTTTACTATG
GGCCAAGTTGTAAGTAGAGAAGGTCAGGGAAGGCAAGAGACGCTCTTCAGGTGCATACGAAGT
ATGCCCAGTCACCCTGATAGAGCATACAACTCTTGCTACAGTGCGGGCGTTTTTCATTTGCACCA
GGGAGATATCCTCAGCGTGATCATCCCAAGAGCACGCGCAAAATTGAACCTCTCACCACACGGT
ACCTTTCTCGGTTTTGTCAAGCTT
Gly/Ser linker (nucleotides 2100-2135 of SEQ ID NO: 45)
(SEQ ID NO: 51)
GGAGGCGGATCAGGAGGGGGCAGCGGCGGGGGCTCT
Truncated APRIL (nucleotides 2136-2543 of SEQ ID NO: 45)
(SEQ ID NO: 52)
CACTCAGTTTTGCATCTCGTCCCTATCAACGCCACGAGCAAGGACGATTCAGATGTGACTGAAG
TCATGTGGCAGCCGGCCCTTCGGAGGGGAAGAGGGTTGCAAGCTCAGGGTTATGGGGTGCGA
ATACAGGACGCAGGGGTGTACCTCCTCTATTCTCAGGTATTGTTCCAAGACGTAACCTTCACGAT
GGGTCAAGTCGTCTCCCGAGAAGGTCAAGGGCGCCAAGAAACCCTTTTTAGGTGCATTAGAAGC
ATGCCAAGCCATCCTGACCGCGCATATAACTCATGTTACTCCGCCGGCGTGTTTCACTTGCACC
AAGGTGATATCCTTAGCGTTATTATTCCGCGAGCGCGGGCCAAGCTGAATCTTTCACCGCACGG
GACCTTCCTTGGGTTTGTAAAACTG
CD8 hinge + transmembrane domains (nucleotides 2544-2750 of SEQ ID NO: 45)
(SEQ ID NO: 53)
ACCACTACCCCAGCACCGAGGCCACCCACCCCGGCTCCTACCATCGCCTCCCAGCCTCTGTCC
CTGCGTCCGGAGGCATGTAGACCCGCAGCTGGTGGGGCCGTGCATACCCGGGGTCTTGACTTC
GCCTGCGATATCTACATTTGGGCCCCTCTGGCTGGTACTTGCGGGGTCCTGCTGCTTTCACTCG
TGATCACTCTTTACTGT
4-1BB co-stimulatory domain (nucleotides 2751-2876 of SEQ ID NO: 45)
(SEQ ID NO: 54)
AAGCGCGGTCGGAAGAAGCTGCTGTACATCTTTAAGCAACCCTTCATGAGGCCTGTGCAGACTA
CTCAAGAGGAGGACGGCTGTTCATGCCGGTTCCCAGAGGAGGAGGAAGGCGGCTGCGAACTG
CD3ζ signaling domain (nucleotides 2877-3212 of SEQ ID NO: 45)
(SEQ ID NO: 55)
CGCGTGAAATTCAGCCGCAGCGCAGATGCTCCAGCCTACCAACAGGGGCAGAACCAGCTCTAC
AACGAACTCAATCTTGGTCGGAGAGAGGAGTACGACGTGCTGGACAAGCGGAGAGGACGGGAC
CCAGAAATGGGCGGGAAGCCGCGCAGAAAGAATCCCCAAGAGGGCCTGTACAACGAGCTCCAA
AAGGATAAGATGGCAGAAGCCTATAGCGAGATTGGTATGAAAGGGGAACGCAGAAGAGGCAAA
GGCCACGACGGACTGTACCAGGGACTCAGCACCGCCACCAAGGACACCTATGACGCTCTTCAC
ATGCAGGCCCTGCCGCCTCGG

### Example 21. TriPRIL CAR Amino Acid Sequences

pMGH71b-TriPRIL CAR (SEQ ID NO: 39):
TriPRIL (truncated APRIL (SEQ ID NO: 40 (amino acids 1-136 of SEQ ID NO: 39))-
Gly/Ser linker (SEQ ID NO: 41 (amino acids 137-148 of SEQ ID NO: 39))-
truncated APRIL (SEQ ID NO: 40 (amino acids 149-284 of SEQ ID NO: 39))-
Gly/Ser linker (SEQ ID NO: 41 (amino acids 285-296 of SEQ ID NO: 39))-
truncated APRIL (SEQ ID NO: 40 (amino acids 297-432 of SEQ ID NO: 39)));
CD8 hinge + transmembrane domains (SEQ ID NO: 42 (amino acids 433-501 of SEQ ID NO: 39));
4-1BB co-stimulatory domain (SEQ ID NO: 43 (amino acids 502-543 of SEQ ID NO: 39));
CD3ζ signaling domain (SEQ ID NO: 44 (amino acids 544-655 of SEQ ID NO: 39))
(SEQ ID NO: 39)
HSVLHLVPINATSKDDSDVTEVMWQPALRRGRGLQAQGYGVRIQDAGVYLLYSQVLFQDVTFTMGQ
VVSREGQGRQETLFRCIRSMPSHPDRAYNSCYSAGVFHLHQGDILSVIIPRARAKLNLSPHGTFLGFV
KLGGGSGGGSGGGSHSVLHLVPINATSKDDSDVTEVMWQPALRRGRGLQAQGYGVRIQDAGVYLL
YSQVLFQDVTFTMGQVVSREGQGRQETLFRCIRSMPSHPDRAYNSCYSAGVFHLHQGDILSVIIPRA
RAKLNLSPHGTFLGFVKLGGGSGGGSGGGSHSVLHLVPINATSKDDSDVTEVMWQPALRRGRGLQ
AQGYGVRIQDAGVYLLYSQVLFQDVTFTMGQVVSREGQGRQETLFRCIRSMPSHPDRAYNSCYSA
GVFHLHQGDILSVIIPRARAKLNLSPHGTFLGFVKLTTTPAPRPPTPAPTIASQPLSLRPEACRPAAGG
AVHTRGLDFACDIYIWAPLAGTCGVLLLSLVITLYCKRGRKKLLYIFKQPFMRPVQTTQEEDGCSCRF
PEEEEGGCELRVKFSRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDALHMQALPPR
Truncated APRIL (amino acids 1-136 of SEQ ID NO: 39)
(SEQ ID NO: 40)
HSVLHLVPINATSKDDSDVTEVMWQPALRRGRGLQAQGYGVRIQDAGVYLLYSQVLFQDVTFTMGQ
VVSREGQGRQETLFRCIRSMPSHPDRAYNSCYSAGVFHLHQGDILSVIIPRARAKLNLSPHGTFLGFV
KL
Gly/Ser linker (amino acids 137-148 of SEQ ID NO: 39)
(SEQ ID NO: 41)
GGGSGGGSGGGS
Truncated APRIL (amino acids 149-284 of SEQ ID NO: 39)
(SEQ ID NO: 40)
HSVLHLVPINATSKDDSDVTEVMWQPALRRGRGLQAQGYGVRIQDAGVYLLYSQVLFQDVTFTMGQ
VVSREGQGRQETLFRCIRSMPSHPDRAYNSCYSAGVFHLHQGDILSVIIPRARAKLNLSPHGTFLGFV
KL
Gly/Ser linker (amino acids 285-296 of SEQ ID NO: 39)
(SEQ ID NO: 41)
GGGSGGGSGGGS
Truncated APRIL (amino acids 297-432 of SEQ ID NO: 39)
(SEQ ID NO: 40)
HSVLHLVPINATSKDDSDVTEVMWQPALRRGRGLQAQGYGVRIQDAGVYLLYSQVLFQDVTFTMGQ
VVSREGQGRQETLFRCIRSMPSHPDRAYNSCYSAGVFHLHQGDILSVIIPRARAKLNLSPHGTFLGFV
KL
CD8 hinge + transmembrane domains (amino acids 433-501 of SEQ ID NO: 39)
(SEQ ID NO: 42)
TTTPAPRPPTPAPTIASQPLSLRPEACRPAAGGAVHTRGLDFACDIYIWAPLAGTCGVLLLSLVITLYC
4-1BB co-stimulatory domain (amino acids 502-543 of SEQ ID NO: 39)
(SEQ ID NO: 43)
KRGRKKLLYIFKQPFMRPVQTTQEEDGCSCRFPEEEEGGCEL
CD3ζ signaling domain (amino acids 544-655 of SEQ ID NO: 39)
(SEQ ID NO: 44)
RVKFSRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
TriPRIL CAR with Whitlow linkers (SEQ ID NO: 57):
TriPRIL (truncated APRIL (SEQ ID NO: 40 (amino acids 1-136 of SEQ ID NO: 57))-
Whitlow linker (SEQ ID NO: 58 (amino acids 137-154 of SEQ ID NO: 57))-
truncated APRIL (SEQ ID NO: 40 (amino acids 155-290 of SEQ ID NO: 57))-
Whitlow linker (SEQ ID NO: 58 (amino acids 291-308 of SEQ ID NO: 57))-
truncated APRIL (SEQ ID NO: 40 (amino acids 309-444 of SEQ ID NO: 57)));
CD8 hinge + transmembrane domains (SEQ ID NO: 42 (amino acids 445-513 of SEQ ID NO: 57));
4-1BB co-stimulatory domain (SEQ ID NO: 43 (amino acids 514-555 of SEQ ID NO: 57));
CD3ζ signaling domain (SEQ ID NO: 44 (amino acids 556-667 of SEQ ID NO: 57))
(SEQ ID NO: 57)
HSVLHLVPINATSKDDSDVTEVMWQPALRRGRGLQAQGYGVRIQDAGVYLLYSQVLFQDVTFTMGQ
VVSREGQGRQETLFRCIRSMPSHPDRAYNSCYSAGVFHLHQGDILSVIIPRARAKLNLSPHGTFLGFV
KLGSTSGSGKPGSGEGSTKGHSVLHLVPINATSKDDSDVTEVMWQPALRRGRGLQAQGYGVRIQD
AGVYLLYSQVLFQDVTFTMGQVVSREGQGRQETLFRCIRSMPSHPDRAYNSCYSAGVFHLHQGDIL
SVIIPRARAKLNLSPHGTFLGFVKLGSTSGSGKPGSGEGSTKGHSVLHLVPINATSKDDSDVTEVMW
QPALRRGRGLQAQGYGVRIQDAGVYLLYSQVLFQDVTFTMGQVVSREGQGRQETLFRCIRSMPSH
PDRAYNSCYSAGVFHLHQGDILSVIIPRARAKLNLSPHGTFLGFVKLTTTPAPRPPTPAPTIASQPLSL
RPEACRPAAGGAVHTRGLDFACDIYIWAPLAGTCGVLLLSLVITLYCKRGRKKLLYIFKQPFMRPVQT
TQEEDGCSCRFPEEEEGGCELRVKFSRSADAPAYQQGQNQLYNELNLGRREEYDVLDKRRGRDPE
MGGKPRRKNPQEGLYNELQKDKMAEAYSEIGMKGERRRGKGHDGLYQGLSTATKDTYDALHMQAL
PPR
Truncated APRIL (amino acids 1-136 of SEQ ID NO: 57)
(SEQ ID NO: 40)
HSVLHLVPINATSKDDSDVTEVMWQPALRRGRGLQAQGYGVRIQDAGVYLLYSQVLFQDVTFTMGQ
VVSREGQGRQETLFRCIRSMPSHPDRAYNSCYSAGVFHLHQGDILSVIIPRARAKLNLSPHGTFLGFV
KL
Whitlow linker (amino acids 137-154 of SEQ ID NO: 57)
(SEQ ID NO: 58)
GSTSGSGKPGSGEGSTKG
Truncated APRIL (amino acids 155-290 of SEQ ID NO: 57)
(SEQ ID NO: 40)
HSVLHLVPINATSKDDSDVTEVMWQPALRRGRGLQAQGYGVRIQDAGVYLLYSQVLFQDVTFTMGQ
VVSREGQGRQETLFRCIRSMPSHPDRAYNSCYSAGVFHLHQGDILSVIIPRARAKLNLSPHGTFLGFV
KL
Whitlow linker (amino acids 291-308 of SEQ ID NO: 57)
(SEQ ID NO: 58)
GSTSGSGKPGSGEGSTKG
Truncated APRIL (amino acids 309-444 of SEQ ID NO: 57)
(SEQ ID NO: 40)
HSVLHLVPINATSKDDSDVTEVMWQPALRRGRGLQAQGYGVRIQDAGVYLLYSQVLFQDVTFTMGQ
VVSREGQGRQETLFRCIRSMPSHPDRAYNSCYSAGVFHLHQGDILSVIIPRARAKLNLSPHGTFLGFV
KL
CD8 hinge + transmembrane domains (amino acids 445-513 of SEQ ID NO: 57)
(SEQ ID NO: 42)
TTTPAPRPPTPAPTIASQPLSLRPEACRPAAGGAVHTRGLDFACDIYIWAPLAGTCGVLLLSLVITLYC
4-1BB co-stimulatory domain (amino acids 514-555 of SEQ ID NO: 57)
(SEQ ID NO: 43)
KRGRKKLLYIFKQPFMRPVQTTQEEDGCSCRFPEEEEGGCEL
CD3ζ signaling domain (amino acids 556-667 of SEQ ID NO: 57)
(SEQ ID NO: 44)
RVKFSRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR

### OTHER EMBODIMENTS

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A chimeric antigen receptor (CAR) polypeptide comprising:
a) two or more extracellular domains, each comprising a Tumor Necrosis Factor (TNF) superfamily receptor ligand or a portion thereof;
b) a transmembrane domain; and
c) an intracellular signaling domain.

2. The CAR polypeptide of claim 1, wherein the transmembrane domain comprises a hinge/transmembrane domain.

3. The CAR polypeptide of claim 1, further comprising one or more co-stimulatory domains.

4. The CAR polypeptide of claim 1, wherein the TNF superfamily receptor ligand is A Proliferation-Inducing Ligand (APRIL).

5.-7. (canceled)

8. The CAR polypeptide of claim 1, wherein the two or more extracellular domains are connected to each other by one or more linker sequences.

9. (canceled)

10. (canceled)

11. The CAR polypeptide of claim 1, further comprising a leader sequence.

12.-16. (canceled)

17. The CAR polypeptide of claim 2, wherein the hinge and transmembrane domain comprises the hinge and transmembrane domain of CD28, CD8, or 4-1BB.

18.-22. (canceled)

23. The CAR polypeptide of claim 3, wherein the co-stimulatory domain comprises the intracellular domain of 4-1BB, CD28, CD27, ICOS, or OX40.

24.-26. (canceled)

27. A CAR polypeptide comprising at least 95% sequence identity with the sequence of SEQ ID NO: 39, 57, 64, or 65, or that is encoded by a sequence comprising at least 95% sequence identity with the sequence of SEQ ID NO: 45.

28.-31. (canceled)

32. A mammalian cell comprising:
a) the CAR polypeptide of claim 1;
b) a nucleic acid encoding the CAR polypeptide of claim 1; or
c) a polypeptide complex of comprising two or more of the CAR polypeptides of claim 1.

33. (canceled)

34. The cell of claim 32, wherein the cell is a human cell.

35. (canceled)

36. A method of treating a cancer, a plasma cell disorder, amyloidosis, an autoimmune disease or disorder, or transplant rejection in a subject, the method comprising:
a) engineering a T cell to comprise the CAR polypeptide of claim 1 on the T cell surface; and
b) administering the engineered T cell to the subject.

37. A method of treating a cancer, a plasma cell disorder, an autoimmune disease or disorder, or transplant rejection in a subject, the method comprising administering the cell of claim 32 to the subject.

38. The method of claim 36, wherein the cancer is BAFF+, B cell maturation antigen (BCMA)+ and/or transmembrane activator and calcium modulating ligand (CAML) interactor (TACI)+.

39. The method of claim 36, wherein the subject is further administered an anti-BCMA therapy.

40.-43. (canceled)

44. A composition comprising the CAR polypeptide of claim 1 formulated for the treatment of cancer.

45. The composition of claim 44, further comprising a pharmaceutically acceptable carrier.

46. A method of treating a subject resistant to anti-BCMA therapy, the method comprising administering to the subject an immune cell comprising a CAR and/or a polynucleotide encoding the CAR, wherein the CAR comprises an extracellular target-binding domain comprising two or more APRIL domains.

47.-79. (canceled)

80. A CAR comprising an extracellular target-binding domain comprising three APRIL domains.

81.-100. (canceled)

101. A CAR comprising an amino acid sequence having at least 90% sequence identity to the amino acid sequence of SEQ ID NO: 39, 57, 64, or 65.

102.-118. (canceled)


SEQUENCE LISTING (67 sequences)

SEQ ID NO: 1 - DNA, 1140 bp, Artificial Sequence
  Synthetic Construct
atggccctccctgtcaccgccctgctgcttccgctggctcttctgctccacgccgctcggccccactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactgaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccaacaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 2 - DNA, 63 bp, Artificial Sequence
  Synthetic Construct
atggccctccctgtcaccgccctgctgcttccgctggctcttctgctccacgccgctcggccc

SEQ ID NO: 3 - DNA, 408 bp, Artificial Sequence
  Synthetic Construct
cactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactg

SEQ ID NO: 4 - DNA, 207 bp, Artificial Sequence
  Synthetic Construct
accactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgt

SEQ ID NO: 5 - DNA, 126 bp, Artificial Sequence
  Synthetic Construct
aagcgcggtcggaagaagctgctgtacatctttaagcaacccttcatgaggcctgtgcagactactcaagaggaggacggctgttcatgccggttcccagaggaggaggaaggcggctgcgaactg

SEQ ID NO: 6 - DNA, 336 bp, Artificial Sequence
  Synthetic Construct
cgcgtgaaattcagccgcagcgcagatgctccagcctaccaacaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 7 - DNA, 1095 bp, Artificial Sequence
  Synthetic Construct
atggccctccctgtcaccgccctgctgcttccgctggctcttctgctccacgccgctcggccccactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactgccatctccagccgacctctctccgggagcatcctctgtgaccccgcctgcccctgcgagagagccaggacactctccgcagatcatctccttctttcttgcgctgacgtcgactgcgttgctcttcctgctgttcttcctcacgctccgtttctctgttgttaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccaacaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 8 - DNA, 63 bp, Artificial Sequence; identical to SEQ ID NO: 2
  Synthetic Construct

SEQ ID NO: 9 - DNA, 408 bp, Artificial Sequence; identical to SEQ ID NO: 3
  Synthetic Construct

SEQ ID NO: 10 - DNA, 162 bp, Artificial Sequence
  Synthetic Construct
ccatctccagccgacctctctccgggagcatcctctgtgaccccgcctgcccctgcgagagagccaggacactctccgcagatcatctccttctttcttgcgctgacgtcgactgcgttgctcttcctgctgttcttcctcacgctccgtttctctgttgtt

SEQ ID NO: 11 - DNA, 126 bp, Artificial Sequence; identical to SEQ ID NO: 5
  Synthetic Construct

SEQ ID NO: 12 - DNA, 336 bp, Artificial Sequence; identical to SEQ ID NO: 6
  Synthetic Construct

SEQ ID NO: 13 - DNA, 1200 bp, Artificial Sequence
  Synthetic Construct
atggccctccctgtcaccgccctgctgcttccgctggctcttctgctccacgccgctcggccccactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactgccatctccagccgacctctctccgggagcatcctctgtgaccccgcctgcccctgcgagagagccaggacactctccgcagatcatctccttctttcttgcgctgacgtcgactgcgttgctcttcctgctgttcttcctcacgctccgtttctctgttgttaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccaacaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccaggacagccacttccaagcagttccagtacaggaaaagaaaaaaaggctcagaagggcaccgtggcgtgcattcgcccagccccagaggttaaagcaccgaaacaatgaactacctgactccctagagcccatatataaaaacatttggaacaaaacatttataggagag

SEQ ID NO: 14 - DNA, 63 bp, Artificial Sequence; identical to SEQ ID NO: 2
  Synthetic Construct

SEQ ID NO: 15 - DNA, 408 bp, Artificial Sequence; identical to SEQ ID NO: 3
  Synthetic Construct

SEQ ID NO: 16 - DNA, 162 bp, Artificial Sequence; identical to SEQ ID NO: 10
  Synthetic Construct

SEQ ID NO: 17 - DNA, 126 bp, Artificial Sequence; identical to SEQ ID NO: 5
  Synthetic Construct

SEQ ID NO: 18 - DNA, 441 bp, Artificial Sequence
  Synthetic Construct
cgcgtgaaattcagccgcagcgcagatgctccagcctaccaacaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccaggacagccacttccaagcagttccagtacaggaaaagaaaaaaaggctcagaagggcaccgtggcgtgcattcgcccagccccagaggttaaagcaccgaaacaatgaactacctgactccctagagcccatatataaaaacatttggaacaaaacatttataggagag

SEQ ID NO: 19 - protein, 380 aa, Artificial Sequence
  Synthetic Construct
MALPVTALLLPLALLLHAARPHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 21 aa, Artificial Sequence
  Synthetic Construct
MALPVTALLLPLALLLHAARP

SEQ ID NO: 21 - protein, 136 aa, Artificial Sequence
  Synthetic Construct
HSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 22 - protein, 69 aa, Artificial Sequence
  Synthetic Construct
TTTPAPRPPTPAPTIASQPLSLRPEACRPAAGGAVHTRGLDFACDIYIWAPLAGTCGVLLLSLVITLYC

SEQ ID NO: 23 - protein, 42 aa, Artificial Sequence
  Synthetic Construct
KRGRKKLLYIFKQPFMRPVQTTQEEDGCSCRFPEEEEGGCEL

SEQ ID NO: 24 - protein, 112 aa, Artificial Sequence
  Synthetic Construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 365 aa, Artificial Sequence
  Synthetic Construct
MALPVTALLLPLALLLHAARPHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLPSPADLSPGASSVTPPAPAREPGHSPQIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 21 aa, Artificial Sequence
  Synthetic Construct
MALPVTALLLPLALLLHAARP

SEQ ID NO: 27 - protein, 136 aa, Artificial Sequence; identical to SEQ ID NO: 21
  Synthetic Construct

SEQ ID NO: 28 - protein, 54 aa, Artificial Sequence
  Synthetic Construct
PSPADLSPGASSVTPPAPAREPGHSPQIISFFLALTSTALLFLLFFLTLRFSVV

SEQ ID NO: 29 - protein, 42 aa, Artificial Sequence; identical to SEQ ID NO: 23
  Synthetic Construct

SEQ ID NO: 30 - protein, 112 aa, Artificial Sequence; identical to SEQ ID NO: 24
  Synthetic Construct

SEQ ID NO: 31 - protein, 400 aa, Artificial Sequence
  Synthetic Construct
MALPVTALLLPLALLLHAARPHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLPSPADLSPGASSVTPPAPAREPGHSPQIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQDSHFQAVPVQEKKKRLRRAPWRAFAQPQRLKHRNNELPDSLEPIYKNIWNKTFIGE

SEQ ID NO: 32 - protein, 21 aa, Artificial Sequence
  Synthetic Construct
MALPVTALLLPLALLLHAARP

SEQ ID NO: 33 - protein, 136 aa, Artificial Sequence; identical to SEQ ID NO: 21
  Synthetic Construct

SEQ ID NO: 34 - protein, 54 aa, Artificial Sequence; identical to SEQ ID NO: 28
  Synthetic Construct

SEQ ID NO: 35 - protein, 42 aa, Artificial Sequence; identical to SEQ ID NO: 23
  Synthetic Construct

SEQ ID NO: 36 - protein, 147 aa, Artificial Sequence
  Synthetic Construct
RVKFSRSADAPAYQQGQNQLYNELNLGRREEYDVLDKRRGRDPEMGGKPRRKNPQEGLYNELQKDKMAEAYSEIGMKGERRRGKGHDGLYQDSHFQAVPVQEKKKRLRRAPWRAFAQPQRLKHRNNELPDSLEPIYKNIWNKTFIGE

SEQ ID NO: 37 - protein, 132 aa, Homo sapiens
VLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFV

SEQ ID NO: 38 - protein, 6 aa, Homo sapiens
KQKKQH

SEQ ID NO: 39 - protein, 655 aa, Artificial Sequence
  Synthetic Construct
HSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLGGGSGGGSGGGSHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLGGGSGGGSGGGSHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 136 aa, Artificial Sequence; identical to SEQ ID NO: 21
  Synthetic Construct

SEQ ID NO: 41 - protein, 12 aa, Artificial Sequence
  Synthetic Construct
GGGSGGGSGGGS

SEQ ID NO: 42 - protein, 69 aa, Artificial Sequence; identical to SEQ ID NO: 22
  Synthetic Construct

SEQ ID NO: 43 - protein, 42 aa, Artificial Sequence; identical to SEQ ID NO: 23
  Synthetic Construct

SEQ ID NO: 44 - protein, 112 aa, Artificial Sequence; identical to SEQ ID NO: 24
  Synthetic Construct

SEQ ID NO: 45 - DNA, 3212 bp, Artificial Sequence
  Synthetic Construct
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaatggccctccctgtcaccgccctgctgcttccgctggctcttctgctccacgccgctcggccccactcagtactccatctcgttcctatcaatgcaacaagcaaggatgattctgacgtgactgaggttatgtggcaacctgccctccgaaggggtagaggtctccaggctcaggggtacggcgtccgcatccaggatgcaggagtttacttgctctatagtcaggttctctttcaggatgtcacattcactatggggcaggttgtaagccgggaaggccaaggtagacaggagactctttttcgatgcatcaggagtatgccttcacatccagaccgagcgtacaattcctgctactctgctggtgttttccatctgcaccaaggtgacattctgtccgtcataattccgagagctagagccaagcttaacctcagcccacacgggacctttctgggcttcgttaagctcggcggaggctctgggggagggtccgggggcgggagtcattcagtgcttcacctcgtcccgattaacgcaacaagcaaagatgactccgacgtgactgaagtgatgtggcagccagcattgaggcgaggtagaggtctccaggctcaaggatatggtgtcagaatacaggatgcaggagtttatctcctgtacagtcaggtgttgtttcaggatgttacttttactatgggccaagttgtaagtagagaaggtcagggaaggcaagagacgctcttcaggtgcatacgaagtatgcccagtcaccctgatagagcatacaactcttgctacagtgcgggcgtttttcatttgcaccagggagatatcctcagcgtgatcatcccaagagcacgcgcaaaattgaacctctcaccacacggtacctttctcggttttgtcaagcttggaggcggatcaggagggggcagcggcgggggctctcactcagttttgcatctcgtccctatcaacgccacgagcaaggacgattcagatgtgactgaagtcatgtggcagccggcccttcggaggggaagagggttgcaagctcagggttatggggtgcgaatacaggacgcaggggtgtacctcctctattctcaggtattgttccaagacgtaaccttcacgatgggtcaagtcgtctcccgagaaggtcaagggcgccaagaaaccctttttaggtgcattagaagcatgccaagccatcctgaccgcgcatataactcatgttactccgccggcgtgtttcacttgcaccaaggtgatatccttagcgttattattccgcgagcgcgggccaagctgaatctttcaccgcacgggaccttccttgggtttgtaaaactgaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccaacaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 46 - DNA, 1184 bp, Artificial Sequence
  Synthetic Construct
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 47 - DNA, 63 bp, Artificial Sequence; identical to SEQ ID NO: 2
  Synthetic Construct

SEQ ID NO: 48 - DNA, 408 bp, Artificial Sequence
  Synthetic Construct
cactcagtactccatctcgttcctatcaatgcaacaagcaaggatgattctgacgtgactgaggttatgtggcaacctgccctccgaaggggtagaggtctccaggctcaggggtacggcgtccgcatccaggatgcaggagtttacttgctctatagtcaggttctctttcaggatgtcacattcactatggggcaggttgtaagccgggaaggccaaggtagacaggagactctttttcgatgcatcaggagtatgccttcacatccagaccgagcgtacaattcctgctactctgctggtgttttccatctgcaccaaggtgacattctgtccgtcataattccgagagctagagccaagcttaacctcagcccacacgggacctttctgggcttcgttaagctc

SEQ ID NO: 49 - DNA, 36 bp, Artificial Sequence
  Synthetic Construct
ggcggaggctctgggggagggtccgggggcgggagt

SEQ ID NO: 50 - DNA, 408 bp, Artificial Sequence
  Synthetic Construct
cattcagtgcttcacctcgtcccgattaacgcaacaagcaaagatgactccgacgtgactgaagtgatgtggcagccagcattgaggcgaggtagaggtctccaggctcaaggatatggtgtcagaatacaggatgcaggagtttatctcctgtacagtcaggtgttgtttcaggatgttacttttactatgggccaagttgtaagtagagaaggtcagggaaggcaagagacgctcttcaggtgcatacgaagtatgcccagtcaccctgatagagcatacaactcttgctacagtgcgggcgtttttcatttgcaccagggagatatcctcagcgtgatcatcccaagagcacgcgcaaaattgaacctctcaccacacggtacctttctcggttttgtcaagctt

SEQ ID NO: 51 - DNA, 36 bp, Artificial Sequence
  Synthetic Construct
ggaggcggatcaggagggggcagcggcgggggctct

SEQ ID NO: 52 - DNA, 408 bp, Artificial Sequence
  Synthetic Construct
cactcagttttgcatctcgtccctatcaacgccacgagcaaggacgattcagatgtgactgaagtcatgtggcagccggcccttcggaggggaagagggttgcaagctcagggttatggggtgcgaatacaggacgcaggggtgtacctcctctattctcaggtattgttccaagacgtaaccttcacgatgggtcaagtcgtctcccgagaaggtcaagggcgccaagaaaccctttttaggtgcattagaagcatgccaagccatcctgaccgcgcatataactcatgttactccgccggcgtgtttcacttgcaccaaggtgatatccttagcgttattattccgcgagcgcgggccaagctgaatctttcaccgcacgggaccttccttgggtttgtaaaactg

SEQ ID NO: 53 - DNA, 207 bp, Artificial Sequence; identical to SEQ ID NO: 4
  Synthetic Construct

SEQ ID NO: 54 - DNA, 126 bp, Artificial Sequence; identical to SEQ ID NO: 5
  Synthetic Construct

SEQ ID NO: 55 - DNA, 336 bp, Artificial Sequence; identical to SEQ ID NO: 6
  Synthetic Construct

SEQ ID NO: 56 - protein, 432 aa, Artificial Sequence
  Synthetic Construct
HSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLGGGSGGGSGGGSHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLGGGSGGGSGGGSHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 57 - protein, 667 aa, Artificial Sequence
  Synthetic Construct
HSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLGSTSGSGKPGSGEGSTKGHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLGSTSGSGKPGSGEGSTKGHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 18 aa, Artificial Sequence
  Synthetic Construct
GSTSGSGKPGSGEGSTKG

SEQ ID NO: 59 - protein, 444 aa, Artificial Sequence
  Synthetic Construct
HSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLGSTSGSGKPGSGEGSTKGHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLGSTSGSGKPGSGEGSTKGHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 60 - protein, 250 aa, Homo sapiens [sequence omitted]

SEQ ID NO: 61 - protein, 15 aa, Artificial Sequence
  Synthetic Construct
GGGGSGGGGSGGGGS

SEQ ID NO: 62 - protein, 20 aa, Artificial Sequence
  Synthetic Construct
GGGGSGGGGSGGGGSGGGGS

SEQ ID NO: 63 - protein, 18 aa, Artificial Sequence
  Synthetic Construct
GGSSRSSSSGGGGSGGGG

SEQ ID NO: 64 - protein, 676 aa, Artificial Sequence
  Synthetic Construct
MALPVTALLLPLALLLHAARPHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLGGGSGGGSGGGSHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLGGGSGGGSGGGSHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 688 aa, Artificial Sequence
  Synthetic Construct
MALPVTALLLPLALLLHAARPHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLGSTSGSGKPGSGEGSTKGHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLGSTSGSGKPGSGEGSTKGHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 4 aa, Homo sapiens
  MISC_FEATURE (2)..(2): Xaa is any amino acid
  MISC_FEATURE (3)..(3): Xaa is Arg or Lys
RXXR

SEQ ID NO: 67 - protein, 4 aa, Homo sapiens
RKRR